SEQUENCE LISTING (8 sequences)

SEQ ID NO: 1 - DNA, 806 bp, Empedobacter Haloabium
gtcgggttcgacgggtacgccaaaggggtcgcgatggcccagggcccgctggtcaacctgatccggtggcaggcttcgtcgcgttcgaagctggcccagcgcgaacgcacgctgcagttctccgccctgggcttcgatgccacgttccaggagatcttcagcgcattgtgctatggcgccagcctggtgctgctggccgagtccatccggcgcgatccgcgcgaactggtgcggctgatgcgccggtacgacgtggaacgcattttcctgccgttcgtcgcgctgcagaacatcgccgaggcggcggtggagctgggcgaaccgttgcctgcgctgaacacgatgatcacggcaggcgaacagttgcgcatcagtcccgccatcgtgcagttcttccgcatgcgcgccggccgcagcctgcacaactactacggcccgaccgagagccacgtcgtgacgacgtatgtgctggacggcgatccgggcgcgtggcccgcgttgccgccgatcggcgcgccgatcgccaacacccagatctacattctcgacgcggcgctgcagccggtggccctgggcgcgcatggcgagctgtatatcgccggcgattgcctggccgacggctacctgaaccggcctgacctgacggcggagcgcttcgtcggcaatgtcttccggccaggcacgcgcatgtacaagacgggcgacatcgcccgctggctggaggacggcaatatcgaatacctgggccgcaacgacagccaggtcaagatccgcggctaccgcatcgagctgggcgaaatcgaggc

SEQ ID NO: 2 - protein, 268 aa, Empedobacter Haloabium
VGFDGYAKGVAMAQGPLVNLIRWQASSRSKLAQRERTLQFSALGFDATFQEIFSALCYGASLVLLAESIRRDPRELVRLMRRYDVERIFLPFVALQNIAEAAVELGEPLPALNTMITAGEQLRISPAIVQFFRMRAGRSLHNYYGPTESHVVTTYVLDGDPGAWPALPPIGAPIANTQIYILDAALQPVALGAHGELYIAGDCLADGYLNRPDLTAERFVGNVFRPGTRMYKTGDIARWLEDGNIEYLGRNDSQVKIRGYRIELGEIE

SEQ ID NO: 3 - DNA, 8397 bp, Empedobacter Haloabium
atgcatacctccgccatacccgacacctgcgcgaccttgttcgacgtcctccgccatcgtgccagcgccgccggcacggcggaccggccggccttcacctatctgaacgatggtgaatcggtcagcggtgcgctcagttatgcccagctcgacgccgcggcgcagcgcctggcggcgcacctgcagcaggtcaccagcccgggcgaccgcgtgctgctcgtgtatccgcccagcctggactacatcgtcgccttctatgcctgcgtgtacgccggtgtcaccgccgtgcccgcgctgccgccggccaatccgcgtgccctgccgcggctgcggctgcaggcggaagacgcccagcccagcgcggccctgaccagcgccgcgatccgcgccacgatcgtcgatggcgcggcgggcgacgacgcgctgcgccgctgccactggctggcgaccgatgcgctggacgagacggcgccgccatggcgcgagccgtcggtgcgtgccagcgacatcgtgttcctgcagtacacctcgggttcgaccggtgcgcccaaaggcgtcatggtgagccatgccagcctgctggccaacgtcgccctcagccagcagctgtacggcatgcgcggcgacgacgtgttcgtctcgtggctgccgccgcaccacgacttcggcctgatcggcacgatcgtctcgccggtctatgtcggctgccacagcgtgcagttcccgcccgccgcgttcctgatgcgcccgcaccgctggctcaagctcatcgcggcataccgcgcccgcatcaccggcgcgcccaacttcgcctaccagttgtgcgcgcagcgcgtcacgccggcgcagcgtgccggcctcgatctgtcctgcctcgaggtcgcggtcaacggcgccgagcgtatccgcatggagacggtacgggagttcgccgccgccttcgccgactgcggcctgaggccggaagcgatggtgccggcgtatggcatggccgagtgtgtgctgctggcttgcgcggcgatggacaagcggccgggcgccttgccgcacagccgccatctcagcaaggcggcgctggagcgcaacgtcgtgaccgacagcgccggcgcggcggacgagatcgagattgcctgcacgggcgcggccgtcaacggcgcgcaccgcatcgtttgcgtcgagccggacagccgcgtggcgctgccggacaacgcggtcggcgaagtctggatcagcggcccatccgtcgccgatggctactggggcaagccggacgccagcgcggcggtattcggcgccgcgctggccggtggccccggccgctggttgcgcacgggcgacctgggattcgtcgccgatggccgcctgtacatcacgggccgcatcaaggaaatgatgatctttaacggccgcaacgtctatccgcaggacgtcgagatcacggtcgagaagctcgataccgctttccggcccagcggctgcgccgtgttcgcggtggaggacgacgccacgaccgcgctggtcgtcgtgcaggagctcgaggcgcgccagcaggcctacacggccacgctggtggcccgactgcgcgaggcgctggccgagcgccacgacatcctcgacctggccggtgtcgtgctggtcaaggcgggccgcattccacgcacctccagcggcaagctgcagcgcgtggcgtgccgccagctgtatctggaaggcgccctcgatcccatctggagctggcgccgtgaagacgacagcgtggccgcggtggcgggtgccgtcgcacccgccgagcagcgcatgctggcgatctggcaggagctgttcgagcaggcgccgctggcgctggacgacaatttcttccgcctgggcggccactcgctgctggcgacccagctgatcggtgccgtcaacgcggcattcggcgtgcagctgccgctgcgggtcgtgttccacgcgccgaccccgcgggcgatggccgcggcggtcggtgacgcggccgcgggcggcgccaccgatgtgctggcgccggccgggcacgcgggtctggcgccgctgtcgttcgcccagcagcgtttctggttcctcgaccagtaccagcccggcaacccgttctacaacatcccgctggcgctcgcgctgacgggcgccgtcgatgccgcactgctggaacgggcgctgaacgcgctggtcgcgcggcatgacacgctgcgtaccagcttccccgccgacggcggcgtgccgcggcagcacgtggcggcgcagctggcgctgccgctgaccatcgtcgacctggccgcgctgccggtcgccgaggccgaggcgcgcaccgaacgcatcgtgcgtgccgaggccgcgcagccattcgacctgacggccggtccattgctgcgagccagtctggtgtcgattgccgatacgcgccatgtgctgctgctgacgttgcaccacatcgtgcacgacggctggtccacgccggtgctgctgggcgaactgcgccgcatctacgcggcgctgcgcgacagtcaggccgcggccctgcctgcgccggccttgcagtacgccgactatgccgtgtgggagcagcgccgctggcagggcgaggcgctggccgcggcgctggcattctggcgcgccaacctggccgacgcctcgccgctgctggcgctgccgaccgaccggccgcgcgccaatgtgatggcgcacgaaggccgggcatggcagacgcgcgtgccggcggcactggtgcgcgacctgaaccggcttgccgccagctcgaacgcgacgctgttcatggtgctgaccgcggcgttgaacgccgtgctgtaccgctattccggccagaccgatttcgccatcggcgccctgtcggccaaccgcccggcaggtaccgagcacatgccgggcaacttcgtcaacgtggtgccgctgcgtgcccgcgtgcacggcgacgatacgttcgcggcgctgcttgccgatacggcggcgaacctgctggccgcctacgactgccagctgccgttcgagttgatcctgcagcacgtggtgtccgagcgcagcccggcctacacgccctatgcgcaggtggtactgaattaccacagcgagttcgaaggccaggaacaggcggcgctggcaccggacggcgacgcgctccacatcgaaggccgccacgcggccagcgtccagtacgcggcgttcgacctgaagatcgagatgaaccgcgtcggcgccgagctggacctggtgttcgagtacagcacggcgctgttcgaccaagcgacgatcgcccggctggccggccactacgtgcgcgtgctcgaacaggtcggcgccgatgcccaggcgcgtgtcgccgcgctggcgctgctgtcggaaggtgagctggcggcgctgtcggcgcagtggcagtccgcccgccacgattacccgcgcacggccaacctggccacgctgctggagcagcaggccgcgcgcacgccggatgcgccggcggtggcttgcgccggcacggtgctgacgtacgcccagttgcacggccgggccaaccgcctggcccacctgctgcgcgcgcgcggcgtcgggccggacgtgctggtgggcgtctgcgtcgagcgttcgctcgacatggtcgtggccgtgctggccgtcgtcaaggccggcggtgcctacctgccgctcgacccgaactatccggccgcgcgcctcgcatacatgctggaagacgccgccccggcgctggtgctgacgcaacagcacctggccgcgcgcctgccggcgcaggcgccggccatcgtgatcgacgccgatcacacggcacacccggacagcgcaccggctccggtgggcgggccggacgacctggcatacgtcatctacacgtccggttcgaccggcaagccgaagggcgccatggtgcagcgccagggcgtgctgaacctgctgacgtggttcgtgcgcgagtacgccatcggcgcggccgatcgcgtgctgctggtgtcgtccttcagcttcgacctgacgcagaagaacatcttcggcatcctgctggtcggcggcgagctgcacctgatggcggacgactacgcgccggaacgcatcggcgcctatgcggggaccgccgggatcacgctgatcaactgcgcgcccagcgcgttctatccgctgctggccgacggcggcgcggcgcgcatggcgtcgctgcgcgccgtcttcctgggcggcgagccgatccaggtcggcctgctgcgcgcggcataccgcgacgtcgccacgccaccactggtgcacaacacgtacggcccgaccgaggcctccgatgtcgtgtcgcactacgcctggcacccgcatgagccggtgacgacgctgccgatcggccgggcgatcgccaacacccgcctgtatgtgctcgatggcggccgccagctggtgccgcaaggcgccgtgggcgagctgtatgtgggcggcgacggggtcgggcgcggctatctgcaccgtcccgaactgaccgccgaacggttcctgcccgatccgtttgccgggcagccaggcgcgcgcatgtaccgcaccggcgacctggtgcgctccctgccggacggcgtgctggaatacctgggccgtatcgatcaccaggtcaaggtgcgtggcctgcgcatcgagctcggggaaatcgaagaggcgctggcggcgctgccggccatcgaccaggcactggtgctcgcctgcgacgatctggccgccgatgtgcgcctggtcgcctacctggtcggcgtcgatgcgcaggccgcgctcgatcccgtcgcactgcgtgcggcgctgacgcaaaccctgccgcagtacatgctgccgtcgcatttcgtccagctgccggcgttcccgttgagccccaacggcaaggtggaccgggccgcgttgccgcgacccgtacaggacctgcatgcaccgttcgtcgcgccgagcggcgccaccgagcaggcgctcgcgcaaatctgggcggaggtgctgaagtgtgccgacgtgggtcgcgccgacgacttcttccagctgggcggccactcgctgctggccacgcaggtgatgtcgcatgtgcgcgcgcgccttggcgtcgacctgccgctgcgcaccctgttcgaatacccgacgctggcggcactgggcgagcagatcgaccgcgccgacaaggccgcgagcggcccgctggccctggccgccggcgacggcgccgcggcgggcgcgttggcgccgctgtcgtatgcgcagcagcgcctgtgggtgctgcagaagctgggcgagaatccggccgtctacaacctgccgttcgccgtcgagctcgagggggcggtcgacgtgcccgcgttgcagcacgcgctggacctgctggcgcggcggcacgcggcgttgcgtaccgccttcgtcaccgtcgacggcgagccgctgtgcgcggtggccgcccatgccgcgttgccgctgcagaccgccagcctggccgacgcggcgccgcaggcggtgcacgactggctggtcgccgcggcgcaggtgccgttcgacctggagtgcgcgccactggcgcgtgcgaccctgttgcacgtcgcgccggcccggcacgtgctgctgctggtcatgcaccacatcattgccgacggctggtcgatcggtgtcctgagccgcgaactgtcggtgctgtacaacgccgcccgccgtggtgtgccggcggcactgccggccttgccgctccagtacagcgattatgcccgctggcagcgcagccgcgcggaagagggcgcgttcgacaatcagctggcttactggcgcgaccgcctggcgcacgcgcccgccatgctggccttgccgctggaccatccgcgtccggccctgccggccctgcgcggcgacgtgctggctttcaccgtcgaaccgggcctgctggcaggcctgcggcgcctggcgcgcgaagggcaggcgagcctgttcatggtactgagcgccgccttcggtgtgctgctgggccgctactccggccagcgcgacctgtgcatcggcacgccgatcgccaaccgccatcatggcgagctggaaggattggtcggcttcttcgtcaacacgctcgtgctgcgcctgacgctcgagccggcgcacggcttcgaggcgctgctggcgcaggtgcgcgaaacggtgctgcaggcattcgccaaccaggacctcccgttcgaacaggtcgtggcggccagcgccggtgcgcgccaggccggccagacgccgctgttccaggccatgctcgcgctgcagaacgcgccgcaggacgaggtggcgctggaggccctgtccggccgcgtgctcgacgtgcacaacggtggcgccaaattcgacctgacgctcgacatcacgccgcgcggcgaccgcctggactgccgctttgaatacgattgcgcgctgttcgaacgcgccacggtggcgcgcttggccgataacctgctcacactgctggccagcatcgtcgccgcgccgcaggcaccgttgcaaacgctggcattgctggcgccagccgagcaggccttgctggcacggctgggcgccggcacgcccgccggcgccgcgccgctggtgcatcgcgcattcgagtcccacgcggcacgcaacccggacgccgtggcattgacgcacgaaggtaccaccctgacgtacgccgaattgaacgcgcgggccgacacgctggcacgcgcgcttacggccgccggggtgggaccggacagccgggtggtcctgtatgccgaacgcggcatcggattgatcaccggtgtgctggcgatcctgaaagctggcggcgcctacgtgccattcgatccggcgtatccgcgcgaacggctggcatacatggcacaggactgcatgccggcggcgctcgtcacggaaccggcgctgctggccgaggcacaggcgctgggaccggccctggcggccgtgccctgctgcctgatcgaagcgggcggcgcgcagcccggcgctgcgccggcgccggcatcgggcgccgccgttggccccggccatctcgcttacatgatctatacctccggctcgacgggacagccgaaaggcgtgcaggtggaacatggcggcctggccagcctggcggcggaccagaaccgggcgctggcgatcggtcccggcagccgcgtgctgcaattcgcgtcgatcagcttcgatgccagcatctgggaaatcgtcatggcgctggccagcggcgcggcgctggtttccgcaccgcgcgccgcgctgatgccgggcgcgccgctgctcgcgttcctgggcgagcagaacatcagccacgcgctgctgccaccttcggtgctggcgatcatggctgacgacgagcggctggcgccgatgacgttgctggtgggcggcgaagcctgcccgccgtccgtcgccgcccactggggccggcgccaccgtttcgtcaacgcctatggtccgagcgagatcacggtctgcgccacgacctggcattacgacggccgcgccggcggcgccattccgatcgggcggccactggcgggtacccgcatccatatcctggacgaggcgggccagccggtaccggtcggcgcggtcggcgagatccatatcggcggcgtcggcgtggcgcgcggttacctgaaccggccggacctgaccgcacagcgcttcctggccgaaccggggcaccccgatacccgcttgtaccgcaccggcgacctggggcgatgggatgcggccggcatgctgcactatgcgggccgcaacgatttccaggtcaaggtacggggcttccgcatcgagctgggcgaaatcgaagccgtgctgcgcgcccagccggcattggccgatgccgccgtgatcgcccgtgcgggggcggacggccagcagcgcctgctggcctatgtggtgccacgcgcggatacggcgcccgaaccggcggccctgcgcagcgccttgctggcacgcctgccggactacatggtgcctggagcgttcatcgcgctgccggcattgccgcagacacccaacggcaagctcgatcgcgatgcgctgccgctgcccgatgacgatgccttggcgcggcaggctttcgtgccgccgcaggacggcatcgagcggcgcctggccgacatctggcaaggcgtgctcggtgtcgcggcggtgggccgtttcgatcacttcttcgagctgggcggccactcgctggcgttgacgaagctcagcttcctggtgcaggaagcgttcggcgtgacgctcagcctgggtcagctctaccagctgcagcagctggcgcagcaggccgaccatatcgccgcggcgcttgccacggcaagccgcaagaaggtgctggtactggacctggacgacgaggaggaagccgcatga

SEQ ID NO: 4 - protein, 2798 aa, Empedobacter Haloabium
MHTSAIPDTCATLFDVLRHRASAAGTADRPAFTYLNDGESVSGALSYAQLDAAAQRLAAHLQQVTSPGDRVLLVYPPSLDYIVAFYACVYAGVTAVPALPPANPRALPRLRLQAEDAQPSAALTSAAIRATIVDGAAGDDALRRCHWLATDALDETAPPWREPSVRASDIVFLQYTSGSTGAPKGVMVSHASLLANVALSQQLYGMRGDDVFVSWLPPHHDFGLIGTIVSPVYVGCHSVQFPPAAFLMRPHRWLKLIAAYRARITGAPNFAYQLCAQRVTPAQRAGLDLSCLEVAVNGAERIRMETVREFAAAFADCGLRPEAMVPAYGMAECVLLACAAMDKRPGALPHSRHLSKAALERNVVTDSAGAADEIEIACTGAAVNGAHRIVCVEPDSRVALPDNAVGEVWISGPSVADGYWGKPDASAAVFGAALAGGPGRWLRTGDLGFVADGRLYITGRIKEMMIFNGRNVYPQDVEITVEKLDTAFRPSGCAVFAVEDDATTALVVVQELEARQQAYTATLVARLREALAERHDILDLAGVVLVKAGRIPRTSSGKLQRVACRQLYLEGALDPIWSWRREDDSVAAVAGAVAPAEQRMLAIWQELFEQAPLALDDNFFRLGGHSLLATQLIGAVNAAFGVQLPLRVVFHAPTPRAMAAAVGDAAAGGATDVLAPAGHAGLAPLSFAQQRFWFLDQYQPGNPFYNIPLALALTGAVDAALLERALNALVARHDTLRTSFPADGGVPRQHVAAQLALPLTIVDLAALPVAEAEARTERIVRAEAAQPFDLTAGPLLRASLVSIADTRHVLLLTLHHIVHDGWSTPVLLGELRRIYAALRDSQAAALPAPALQYADYAVWEQRRWQGEALAAALAFWRANLADASPLLALPTDRPRANVMAHEGRAWQTRVPAALVRDLNRLAASSNATLFMVLTAALNAVLYRYSGQTDFAIGALSANRPAGTEHMPGNFVNVVPLRARVHGDDTFAALLADTAANLLAAYDCQLPFELILQHVVSERSPAYTPYAQVVLNYHSEFEGQEQAALAPDGDALHIEGRHAASVQYAAFDLKIEMNRVGAELDLVFEYSTALFDQATIARLAGHYVRVLEQVGADAQARVAALALLSEGELAALSAQWQSARHDYPRTANLATLLEQQAARTPDAPAVACAGTVLTYAQLHGRANRLAHLLRARGVGPDVLVGVCVERSLDMVVAVLAVVKAGGAYLPLDPNYPAARLAYMLEDAAPALVLTQQHLAARLPAQAPAIVIDADHTAHPDSAPAPVGGPDDLAYVIYTSGSTGKPKGAMVQRQGVLNLLTWFVREYAIGAADRVLLVSSFSFDLTQKNIFGILLVGGELHLMADDYAPERIGAYAGTAGITLINCAPSAFYPLLADGGAARMASLRAVFLGGEPIQVGLLRAAYRDVATPPLVHNTYGPTEASDVVSHYAWHPHEPVTTLPIGRAIANTRLYVLDGGRQLVPQGAVGELYVGGDGVGRGYLHRPELTAERFLPDPFAGQPGARMYRTGDLVRSLPDGVLEYLGRIDHQVKVRGLRIELGEIEEALAALPAIDQALVLACDDLAADVRLVAYLVGVDAQAALDPVALRAALTQTLPQYMLPSHFVQLPAFPLSPNGKVDRAALPRPVQDLHAPFVAPSGATEQALAQIWAEVLKCADVGRADDFFQLGGHSLLATQVMSHVRARLGVDLPLRTLFEYPTLAALGEQIDRADKAASGPLALAAGDGAAAGALAPLSYAQQRLWVLQKLGENPAVYNLPFAVELEGAVDVPALQHALDLLARRHAALRTAFVTVDGEPLCAVAAHAALPLQTASLADAAPQAVHDWLVAAAQVPFDLECAPLARATLLHVAPARHVLLLVMHHIIADGWSIGVLSRELSVLYNAARRGVPAALPALPLQYSDYARWQRSRAEEGAFDNQLAYWRDRLAHAPAMLALPLDHPRPALPALRGDVLAFTVEPGLLAGLRRLAREGQASLFMVLSAAFGVLLGRYSGQRDLCIGTPIANRHHGELEGLVGFFVNTLVLRLTLEPAHGFEALLAQVRETVLQAFANQDLPFEQVVAASAGARQAGQTPLFQAMLALQNAPQDEVALEALSGRVLDVHNGGAKFDLTLDITPRGDRLDCRFEYDCALFERATVARLADNLLTLLASIVAAPQAPLQTLALLAPAEQALLARLGAGTPAGAAPLVHRAFESHAARNPDAVALTHEGTTLTYAELNARADTLARALTAAGVGPDSRVVLYAERGIGLITGVLAILKAGGAYVPFDPAYPRERLAYMAQDCMPAALVTEPALLAEAQALGPALAAVPCCLIEAGGAQPGAAPAPASGAAVGPGHLAYMIYTSGSTGQPKGVQVEHGGLASLAADQNRALAIGPGSRVLQFASISFDASIWEIVMALASGAALVSAPRAALMPGAPLLAFLGEQNISHALLPPSVLAIMADDERLAPMTLLVGGEACPPSVAAHWGRRHRFVNAYGPSEITVCATTWHYDGRAGGAIPIGRPLAGTRIHILDEAGQPVPVGAVGEIHIGGVGVARGYLNRPDLTAQRFLAEPGHPDTRLYRTGDLGRWDAAGMLHYAGRNDFQVKVRGFRIELGEIEAVLRAQPALADAAVIARAGADGQQRLLAYVVPRADTAPEPAALRSALLARLPDYMVPGAFIALPALPQTPNGKLDRDALPLPDDDALARQAFVPPQDGIERRLADIWQGVLGVAAVGRFDHFFELGGHSLALTKLSFLVQEAFGVTLSLGQLYQLQQLAQQADHIAAALATASRKKVLVLDLDDEEEAA

SEQ ID NO: 5 - DNA, 13893 bp, Empedobacter Haloabium
atgaaactccatgaactgatctcccatctgcatgccaccggcgtctcggtgcagaaccgcgacggcaagctgcaggtgacgagcgccgacggcgacctgcccgacgccacgctggcggcgctgaagaagcacaagaaggacgtggccgcatactatgccgagcccgcgccggtcgatgtcgcggcaccggaacgggagcagccactttcgttcgcgcagcgccgcctgtatttcctgtaccagtacgagccggccgcgacgcacttcaacctgccgatggagctcggcatcgagggcgccctcgacagcgagcgcctgcgcggcgcgctgctcgacgtggtgcagcgccatcccatctaccgcaccacgtatggcatgcgcgacggcgtgccattccagcgcgtgcgcagcgacctgcagcccaccctcgggctggacgacctgcgccacctcgatgccgccgctgccgatgaacggatggcgctgcagcgcgcacgtattgccgccacgccattcgacctggccaacgagctgccgctgcggatgcacctgttccgccagggcgaggcggcgtattcgctgctgatcgtgttccaccatatcgcgaccgacgaatggtcgatccagcagctgatgcgcgaactgtcggacgcctatcggggcaccggccccgccgcgccggtgccggcgtacggtgaatacgtcgcctggcagaacagccggcatgcggggcgcggctacgaagcggcccggtcctactggaccgaacacctggccgacgcggagcccgtgctggcattgccggcggaccgcgcgcgcccgtcacgccagacctaccgcaccggcctcgagcggcttgcgttgccggcggccttgcgcgaacgcgccagccagtgcgccggccggctcggcatctccgagttcgcgctgtatctcggcctgtaccaactgctgctgcaccgcctgacggggcagcgcgacctcgtggtcggcacggacgtgttcggccgcgatcacggccggttccgcgaggtggcgggcttcttcgtcaatcagctggcactgcgccagcaggtcccggccggcgcccaggccgatgaattcctgcgccaggtggcgcgcgacgtcaacgatgcgatgctgttccaggacctgccgttcgaccagctggtcgacgctttgcaggtggagcgcgacccggcctattcgccgctgttccaggtgaagttcctgtaccgccgcaacagcctgacgccggacctgttcgacggcctgcgcagctggaacaaggagatgttcgcggtacagtcccagtacgacctgacgctgcaggtgctgccggacacggtggaagcgtatttcaacccggacctgttcgacgcggcgcgcgtggccggctggctggaactgtatgtggcgctggccgaggaggtcgtggccgacccggcgcagccgcttgccggcctgctcgatgcgcgcctgcgcgccatggtcgcaccgttcagccatggcgaggcgaccggcccggccgggctggcgctgtgcgaccgcatcgccagctgggcgggtgccacgccggagcgtgtcgccatcggcagcgccgaaggcgacctgacgtacgccgaactggtacgccgcatggaggccgtggccgggcaactggcggcgctgggcaccggccgcggcgacaaggtggcggtctatctcgaccgttcggccgacctggtggtcgccgtgctggcgatcgcccgcgtgggcgcggtgctggtgccgctcgacacggacaatccaccggagcacatcgcgttcgtgctgcacgacagcggtgccaacgtggtgctgagcgaaagcctgcgggccgacgacatcgtcgatttctatgggctgtggctggacatcggcgcgctgagcgcggcgccggcaccgcaggcgctgcccgcatacgacacgctgcaaggcgacgacctggtctaccagctgtacacctccggctcgacggggcggccgaagggcgtgctcgtcacgcgcgccggcttcgccaatctgtgcgactggtatgcctcgttcgcccgaatcggccccgacagcccggtgctgttgatgattccgatcggcttcgacgcttcgctgaagaacatcttcacgcccttgatgcagggcgcgacgctggtgctggcaccggcggcgccgttcgatccggatgccctgctggcgctgatcgccagccgcggcgtggccgtggtgaacacggcgccgagcgcgctgtatgcgctgctgcagcaggacgcgccgcgccagtacgcggcgctggccgggctgaccatgttcgccgtcggcggcgaggcgctggacctggggctggtacgcccgtggctggacagcccgaactgccgtgcgctgctggccaatatctatggcccgaccgagtgcaccgatatctcgctggcgttcgcggccgatgccgcgacctggctggcgcgcgccacggtgacgatcggccggccgatccgcaacacccaggctttcatcgtgaacgacgagctggcgctgtgcccacccggcacgccgggcgaactggtgattgccggctgcggcgtcgcgcgcggctatcaccagctgccggacgcggatgcgcgcagcttcgtgcacgccgcgctggcacaggggcgtatctatcgcaccggcgactatgcctgccatgaggccgacggcaatgtgctgtacctgggccgccgcgacggccagatcaagatccgcggcaagcgggtggagacgggcgaagtgctggcgcaaatggcgcgcctgctgccgggccgcacgctgagcgtgcagcgctatgcgcgcgaccgcgtcgagatgctggtgggcttcgtggcgggcctgccgtccgatctggacagcgtgcagctgcgtgccgaactggcgcgccacctgccgcgccacgcggtgccggccgatatcgtcttcgtgccgtcgatgccgctgagtgccaacggcaagatcgcggcggcggcgttgctggcgctgtacgaggaacaccgcagcacccgccagtccgccacgcgcgcgctgagtgcgaccgaagcggccatcgccgcgatctggcaccagttgctgggcgaggtcgcggtggaggcggacagcagcttcttcgccgtcggcggcgactcgatcttctcgatccagctggtggcggaattgcagaagctggggtacgcggtcgcggtggccgacatcttcaaatacccggtactggaacagctggccgcgttctgcgacagtcgttcgcatgtcgccgtcacgaccacggaggcgctcgcaccgttcgcgctggtcgacccggccgacctggccgctctgccggaagggctggaggatgcctacccggtcacgtcgctgcagcaggggatgctgttccactgccggatggagccggacagtgcgatgtaccacgatgtcttcagctacgagctgcgtttcgactacgatgccgccctgctgaagcaggccgtggggctggtgctggcccacaaccaggtgctgcgtaccggcttcgaactcgataccgtgtccgagccgctgcaactggtgtatgcgcgcgtcgagccggagtggtcggagcaggacctgcgccacctgtccgcggcggagcaggaggcggcggtggccacggccatcgcttcgctcaagcgcaccggtttcgccctgtccggcccgagcctgatccgcttcaccgtgttgcgcaaggccgagggctgcatccagctgctgatcgacgcgcaccacgcgatcctggacggctggagcatggcaacgctgcagcggcagatcttcgagcactacggccatctgcgcttcggcctgccgctggccgacgtcttcgacacgggcgggttgcggttcgccgactacgtcgcccagcaggccgcggccgagcaggacgacgccgcggccgcgcactggcgtacgtattgccgcgccgccggcagcggcgcgctgtcggcgcggctgccgcaacagggcgaagcggtgttgcacacgctgcccttgccggcggacctgcccgcacgcctggcgcaacgtgtcgcgaccgatggcgtgatgctgaaaacgctgctgatgatggcgcacgcgtacatgctgcgcgcgctcctgccgagtgagcgcctcagcacggcgctgacggacaacggccggcccgaaacgccgggcgcgcagaacatcgtcggcctgttcgtcaacgtgctgccggtggccttcgacctggacgccagctggcgccagctggccgccgcgttgcaggcggacgaggtggcgcgcaagccgttccggcgcttcccgttcgcgcacatcgtgcgcgaacaacgggcgctgcagatcgacacgctgtttacctacaataacttccatgtcagcgaggcgctgcaggcggccgagtggctgcagatcgagccgggcaacagctatgaggaaaccaatttcaagctggcggtgctggtcaacggcaacctgcagagcggcctgacgctgacgctcgaaagccgcctggcgctgacggcggcgcaggtcgcaacgctgcagcgcgagttcgtgttcgccctcgactgcatggcacaggcgttcgacgcgccgatcccgcagcgtgccgatcgcctgctgcccgtgctggcgcaggccggtgcggcagtggcccggttgcgctggcagggcgtcgccccggcggcggtgctggaggcggcgctggcccgttgcgccctgcgtgtcgcggcaatcgagcgcgcgccggcacaggcgccgttcgatatcgccgccagcgtggagcaggacggccagcggctggagtggcggatcgcgccggagtgggcgcagcatcccgacctgccggccctgctgtccgaaacgatggaacgcgtgctggcgacaggtgcgcccgcgggcgacgtcgccgtcgcttgcgatgcgcagggagcggcatggccgctgcgccagctggaagacgacatggcgttctggcggggccggctggccgaagcgccagcgcacctgaacctgccgcaaacgctggcgctggccgcgggcgcggaacgcacggacgagcggcatgtgcgggccgtcgatacggcggcgctggcggccctgaccgcgcgcaccgggctgtcccgcggcgccatcctgctgggggcatggctggcactgctggcgcgcctgagcgggcaggaaaccgtgctgaccggcgtacgcctgcgcgccggcggaccgttgctgccgctggtggccgagaccggcgacgacccggctgcaacggtcctgctgacgcgtgccgctggcgcgctgcaggcctgcgccgcacacgccggcgtgcccgccagcctgctgccggcacgccatgcggccgcgttcgcgctggccgatgacggcccgctgccggccgacatggcgatcgtcgcgaccgacgacggcgcctgccgcctcgaactggcggccgatgtccatgacgccgccggcgccgaccggctcgcggccaacctggccgagctgttgcaaggcgccgccgccgcgccgggcgagcggctgtcgcgcctgccgctgctgggcgcggcggagcgccaccgcgtgctggtgcaattcaacgacagcgcccagcacttcgacgacacccgccagttgcaccagatggtcgaagaccaggccgccgccgatcccggcgcgctggccctgctgtacggcagcgacacgatgacgtacgaggtgctgaaccgccgtgccaaccaggtggcgcaattcctgcacggccatggcatcggtgccaacgaccgcgtcgccgtctgcatggagcgtggcctggagatggtggtcgcgatcctcggcgtgctcaaggccggcgccgcctacatgccgctcgacccggcctatccggtcgagcgtatcgcctatatgctcgacgacagcgcgccccgggcgctgctggcccaggcgccgctgctggcggccttggagccggtgcgccggctggcggccgagctgccttgcctgctgctggccgaaggcctggcggtgctggacgggctgccggatgcgaacccgcccgcgccgccgctggcgcaggccgcagccaacctgatgtacgtgctgtacacgtccggctcgaccggccggcccaaaggggtcgcgatggcccagggcccgctggtcaacctgatccggtggcaggcttcgtcgcgttcgaagctggcccagcgcgaacgcacgctgcagttctccgccctgggcttcgatgccacgttccaggagatcttcagcgcattgtgctatggcgccagcctggtgctgctggccgagtccatccggcgcgatccgcgcgaactggtgcggctgatgcgccggtacgacgtggaacgcattttcctgccgttcgtcgcgctgcagaacatcgccgaggcggcggtggagctgggcgaaccgttgcctgcgctgaacacgatgatcacggcaggcgaacagttgcgcatcagtcccgccatcgtgcagttcttccgcatgcgcgccggccgcagcctgcacaactactacggcccgaccgagagccacgtcgtgacgacgtatgtgctggacggcgatccgggcgcgtggcccgcgttgccgccgatcggcgcgccgatcgccaacacccagatctacattctcgacgcggcgctgcagccggtggccctgggcgcgcatggcgagctgtatatcgccggcgattgcctggccgacggctacctgaaccggcctgacctgacggcggagcgcttcgtcggcaatgtcttccggccaggcacgcgcatgtacaagacgggcgacatcgcccgctggctggaggacggcaatatcgaatacctgggccgcaacgacagccaggtcaagatccgcggctaccgcatcgagccgggcgaggtcgaggcggcactggccgcgtgcgccggcgtgcgcgaggcggtcgtggtggcgcgcgaagacgtgccgggacagaagcgcctggtggcgtatctgctggcccagccaggccacacgctggcaccggcggcgctgcgcgaccggctggccaccgtgctgccggactacatggtgccggccgcctttgtctgcatgacggcgttccccgtcagcccgaacggcaagctggaccggcgcgcgctgccggcgcccgacgccgccgcgcaattgcgccagccgtacgaagcgccgcaaggaagcaccgaaacggcgctggcggcgatctgggaagacctgctggccgtacgcgacgttggccgccgcgaccacttcttcgaactcggcggccactcgttgctggccgtgcggctgaccacgcgcgtacgccaggtactgcagcgtgagctggcgctgcgggcgttgttcgagcagccggtgctggccgatctcgcccgcgtcgtcgatggcctggacagcgccggtaccgcaccgctgcgcgcgttgccgcgtacgcccgaccaggtgctgcccctgtcgttcgcgcagcagcgactgtggttcgtgcaggagctcgaaggtcccacgccgacctacaacatgccggccgcgctgcgcctgacggggcggctggatgccgccgcgctggagccggcgctgcaatacctgatcgagcgccacgaggtcctgcgcaccaacttcgacagcgtggagggcgtgccgcacctgcgcatcgcgccgtcgcgtaccgtgacgctggccgttaccgacgtcgcgccggacgaggtggaggcgcgtgccgcgcgccatgcggcgctgccgttcgacctggcgcgcgagcccttgctgcgtgccgaactgctgcggctgtcggccgaccagcacgtgctgctgctgaacgtgcaccatatcgtcagcgacggctggtcgctgaacatcctggccgacgaatggctgcgtgcgtacgacgccctgcgcgccggccgcgcgccggcgctgccggtgctgccgctgcagtacgccgactatgcgtactggcagcgcgaacaactgaccgaagccgtgcgcgagcgccagctggcctattggaccgggcaactggccggtgcgccggagctgctcgacctgccgaccgaccgcgtgcggccggcggtgcagcgcttcgatggcggcgatgaacagctgcggctggacccggcgctgtcgcacgccgtgcgccagctggggcatgcgcacaatgccagcctgttcatgacgctggtcacggcgttcggcctgctgctgggccgtctcagcggccaggacgacgtgctggtcggcgtgccgcaggccacccgcgaccggcgcgagctggagggcatgctcggcatgctgctgggtaacctggtcctgcgcatgcgcctggacgacgcggccggtttcggcacgctgctggagcaggtgcgccgcaccgcgctggaggcttacgaacacagcgccatcccgttcgagcaggtcgtcgacgcgttgccgctgcagcgtgacctgagccgcaatccgctgttccaggtcttcttcaacatgctcaacctgccggagacgaactatacgtcgccggagctggcgatcgaaggactgcaaagcacgctgctggacgccaagttcgacctgacgctgtatgcgcaggacagcgaagaaggcatcctgctgcacctggtgtacaaccgtggcctgttcgatgcgcagcgcatgcgcgaattgctgcggcagtaccacagcttgctggagcaggtcagccaggcgcccgccatcgcctgcaaggccgtgtcgctgctgacggcgccagcgcgcgcggtcctgcccgatccggcggtcgtcctggatgcgacctggcacggcagcattcccggccgctttgccgcgctggtggcggcgcagccggcggcgctggccgtcacggcggcgcacctgcagtggacctacgcggaactggacgagcgcagcgaggccgtggcctgctggctgcaggaggccggcgtcggcgccggcgccgtggtggcgatctgcgccgcccgccgcgcggcgctggtgccggccgtgctgggcgtgctgaaggcgggtgccgcctataccatcgtcgatcccgcttacccggccgagcacgtgcgcgcctgcctggccgtggcccggcccgccgcgtggctgacggtggccgagggcggcgatgccgcattgcttgcctgcctgcccgcgccggtgccgcgactcgatctgagcgggaacgatggctggccggtgctggcagcgggcgtgcgtgccgtgccggccgcctggacggccgacgacgtcgccgtgctgacgttcacgtccggctccaccggcctgcccaaggccgtcgaaggccgccacggcgcgctgacgcacttctacccatggctgcaacaacacttcggcatggggccgcaggatcgctacgcactgttgtcgggcctcgcgcacgacccgctgcagcgcgatatcttcaataccttatggatgggcgccagcctgcacgtgccgccggtggacgccatcggcccgggcctgctggccgactggatggcggccgagaacatcagtgccgtcaacctgacgccggccatgctgcagctgctgtgccaggacgcacgcgctctgccgacattgcggcatgccttcctggtgggcgatatcctgacgcaggccgacgtggccctgctgcagcaggtggcgccgcgctgcgccgtggtcagctactacggcgccaccgaggcgcagcgggcgttcggcatggtggagatcgccccgggtacggcggctggcctgacgcgcgacgtcatcgcgctgggccacggcatccccggcgtgcagctgctggtgctgaacggcgccggcacgctggccgggatcggcgaggtgggcgaagtgtgcatccgcagcccgcacctggcgcgcggctaccgcgacgacgcggcgatgacggcacgccagttcgtcgccaacccgttcggtggcggcgaccgcctgtaccgcacgggcgacctgggacgctatctgcccgacggcatggtggcgggcctgggccgcaacgaccagcaggtcaagctgcgcggcttccgcatcgagctgggccacgtcgaggccgcgctggcccggctgccgcaagtgcgcgaagccgtggtgctggcgttgggcagcggcgaggcgcgtcgactggtcgcatacgtcgtcccgcgcggcaccttcgatgccgacgcggccgcggcggccttgcgcggcaccttgcccgactatatgcggccggccgcctacgtggtcctcgagcgtctgccgctgacgcccaacggcaagctcgatcgtcgtgcgctgcccgcgccggcggccacgcccgcggtggcggacacggcgccggcgacggcactggaagcctcgctgtgcgcgctcatggccgagctgctgaaccgcgacgcggtcggtccggccgagaatttcttcgcgctgggcggccattcgttgttggcgacgcgcctggtatcgcgcatccgcgcagcctgcggcgtgcagttgccgctgcgcgccgtgttcgaggaacccacgccggcggcgctggcgcggctggtggaacgggccggcggcgacaacgccgggccggcgccgcgcgaacgctcgggctggcatccgctcagctcgcagcagcagcgcctgtggttcctcgaccgcttcgagcccgccaacccgttctacaacatcccgctcgcgctgcgcctgcgcggcacgttggtgccggcgcagctgcagcaaagcctcgatgcgctggccgcgcgccatccgtccctgcgcacccgcttcgccacgcaggacggccagccggtacaggaaatcctggcaccggcagcggtgccgctggcgctcacggacctgacgggactggctccggcgcagcgcgaggaggcggcccggcgcgccgccgccaccgtgacgctgcagccgttcgtgctggaacagggcaatctgctgcgtgcggcgctgctgcggctggacgatgccgaccatgtgctggtactggtggtccatcacatcgtcagcgatggccgtmwcgctggcggtgctcgccgacgaactcgcggcgtgtaccgcgccggcacgaccggcggcgccgcggcgctgccgccgctgccattgcactacagcgatttcgcgcactggcagcgcgactggctgcagcagccggccgcgctgcgccagctggcctactggaacgctcaactggccgacgcgccggccgtgcacgcgctgccgctggaccggccgcgcccggccatccagagctatcgcggcgcgacgcacggtttcgccatcggcgccgcgacgctggccgggctgcgtgagctggcagccgcgcaggcggaaccgaccacgctgttcatggtgctgtgcgccgccttcaatgtgctgctgtaccgtcacagcggccaggccgacctgtgcatcggtaccccgatcgccaaccgccagcacgacggcctggaccgggtggtgggcttctttgccaacacgctggtgctgcgcagccggccggctcccggccagccgttccagcagttcctgcgcgacgttcgcgcgacggcgctggacgcctacgccaaccaggacatcgccttcgaacgcgtggtggaggcggtcaagccgcaacgtcataccagccatgcgccgctgttccaggtcatgctctccctgcaggagtcgctggccctgccgcaggtggacgatacgctgcggctggaagcgctcacgctggacagttccgtggcgcgcttcgacctgacgctcagcctggtggaggaaggcggcacgctgctggcggcgttcgagtacaacaccgacctgttcgacgccgcgaccatcgagcgctgggccggccacttcagccacctgctcgatgcggtggtggccacgccgcagctggcgctggatcgcctgccgttgctggacgacgccgagcgtcgtgacgtactgctggccagcgccggcgagcgcgccggcccggtcggcgacaccgtgctgcatgcgctgttcgaacagcaggcgctggcgcatccgcagcgttgcgcggcgcaggccggggccgccagcatcacctatggtgagctcaatacgcgtgccgccgagctggcattgcggctgcgccacgccggggtcgcagcgggcgaccgggtggcggtgcacgcgcagcgctcgctcgagctgctggtcgcgctgctcggcgtgctgaaggccggtgccgcctacgtgccgctcgatccggcacagccgcaggaacggctcgctcatatgctgcgcgacagtgcgccggccgccgtgctgacccagcaggggctggccggtggcgcgctgctggcaagtgtcccgtgccgtgtgttgttactggacgggccagccgccgccgcacccgcgccgctggcggacgtgctcgtacaaccgcacgacctggcgtatgtgatgtacacgtccggttcgacgggtatgccgaagggcgtgatggtcgaacatgccagcatcgtcaacacggtgcgcgcgcatgtgcggcaatgcgcgctgcaggcccaggatcgcgtgctgcagtttgtctcgtacggcttcgacgtctcggccggcgagatcttcggcgcgttcgcggccggcgccacgctcgtgctgcggccggacgagctgcgcgtgccggacgaagcgttcgccgccttcctgcgcgagcaggccgttaccgtggccgacctgccggcggcgttctggcaccagtgggtgcacgagatcgccgccggccgcagcttgccggggccggcgttgcggctcgtcctggccggcggcgaaaaggccgacgtggcgcgcctgcgcacctggctgaccctgccggcaacgcggcacgtacgctggatcaatgcctatggccccaccgagaccacggtcaacgcgagttacatgccgtatgacgcgctgtccgagccgccagccggcgaggtgccgatcggccggccgatcgacaataccgtcgcgtatgtcctcgacgcacacctgcagccggtagccttcggtatcgccggcgagatctacctcggcggcgctggcgtggcgcgcggctacctgaaccagccggaactgaccgaacgcgcgtttgtcgccgatccgttcgccggcggcgcggcgcgcatgtaccgctccggcgacctgggacgccggctggacgacggtacgctcgaatacctgggccgtaacgacagccaggtgaaattgcgcggctaccgcatcgagctgggcgaaatccagtcgcgcctggccacgctggacggcgtgcgcgaggcatgcgtcatgctgcgcgaggtggccggcacaccgcgcctggtggcttacctggcggcggcggagggcatgcagctgtccgctgcggagctgcgtcgcatgctggccgccagcctgccggactatatggtgccgtcggccttcgtctggctgccggtcctgccggtcaatgccagtggcaaggtcgagacggcggcgttgccggaaccggggcccgccgacatggaagcgcgcgtgatcgaaacgccggtgggagcgcgcgagcagctgctggcgcagatctggcaggacttgctggcattgccgcaggtgagccggcaggatcacttcttcgaactgggcggccactcgctgatggtggtgaccttgatcgaccgactgcatcaacacgacctgcatgtggacgtgcgtaccgtattttccagcccgacgctggcggcgatggcggcggccctggccgaccgcgccggcgcgacggccgcctttgtcgcaccaccgaacctgattccgggcgaatttgccgcctcggcctccaccgatcaagccaactttgaagagtttgaactatga

SEQ ID NO: 6 - protein, 4629 aa, Empedobacter Haloabium
MKLHELISHLHATGVSVQNRDGKLQVTSADGDLPDATLAALKKHKKDVAAYYAEPAPVDVAAPEREQPLSFAQRRLYFLYQYEPAATHFNLPMELGIEGALDSERLRGALLDVVQRHPIYRTTYGMRDGVPFQRVRSDLQPTLGLDDLRHLDAAAADERMALQRARIAATPFDLANELPLRMHLFRQGEAAYSLLIVFHHIATDEWSIQQLMRELSDAYRGTGPAAPVPAYGEYVAWQNSRHAGRGYEAARSYWTEHLADAEPVLALPADRARPSRQTYRTGLERLALPAALRERASQCAGRLGISEFALYLGLYQLLLHRLTGQRDLVVGTDVFGRDHGRFREVAGFFVNQLALRQQVPAGAQADEFLRQVARDVNDAMLFQDLPFDQLVDALQVERDPAYSPLFQVKFLYRRNSLTPDLFDGLRSWNKEMFAVQSQYDLTLQVLPDTVEAYFNPDLFDAARVAGWLELYVALAEEVVADPAQPLAGLLDARLRAMVAPFSHGEATGPAGLALCDRIASWAGATPERVAIGSAEGDLTYAELVRRMEAVAGQLAALGTGRGDKVAVYLDRSADLVVAVLAIARVGAVLVPLDTDNPPEHIAFVLHDSGANVVLSESLRADDIVDFYGLWLDIGALSAAPAPQALPAYDTLQGDDLVYQLYTSGSTGRPKGVLVTRAGFANLCDWYASFARIGPDSPVLLMIPIGFDASLKNIFTPLMQGATLVLAPAAPFDPDALLALIASRGVAVVNTAPSALYALLQQDAPRQYAALAGLTMFAVGGEALDLGLVRPWLDSPNCRALLANIYGPTECTDISLAFAADAATWLARATVTIGRPIRNTQAFIVNDELALCPPGTPGELVIAGCGVARGYHQLPDADARSFVHAALAQGRIYRTGDYACHEADGNVLYLGRRDGQIKIRGKRVETGEVLAQMARLLPGRTLSVQRYARDRVEMLVGFVAGLPSDLDSVQLRAELARHLPRHAVPADIVFVPSMPLSANGKIAAAALLALYEEHRSTRQSATRALSATEAAIAAIWHQLLGEVAVEADSSFFAVGGDSIFSIQLVAELQKLGYAVAVADIFKYPVLEQLAAFCDSRSHVAVTTTEALAPFALVDPADLAALPEGLEDAYPVTSLQQGMLFHCRMEPDSAMYHDVFSYELRFDYDAALLKQAVGLVLAHNQVLRTGFELDTVSEPLQLVYARVEPEWSEQDLRHLSAAEQEAAVATAIASLKRTGFALSGPSLIRFTVLRKAEGCIQLLIDAHHAILDGWSMATLQRQIFEHYGHLRFGLPLADVFDTGGLRFADYVAQQAAAEQDDAAAAHWRTYCRAAGSGALSARLPQQGEAVLHTLPLPADLPARLAQRVATDGVMLKTLLMMAHAYMLRALLPSERLSTALTDNGRPETPGAQNIVGLFVNVLPVAFDLDASWRQLAAALQADEVARKPFRRFPFAHIVREQRALQIDTLFTYNNFHVSEALQAAEWLQIEPGNSYEETNFKLAVLVNGNLQSGLTLTLESRLALTAAQVATLQREFVFALDCMAQAFDAPIPQRADRLLPVLAQAGAAVARLRWQGVAPAAVLEAALARCALRVAAIERAPAQAPFDIAASVEQDGQRLEWRIAPEWAQHPDLPALLSETMERVLATGAPAGDVAVACDAQGAAWPLRQLEDDMAFWRGRLAEAPAHLNLPQTLALAAGAERTDERHVRAVDTAALAALTARTGLSRGAILLGAWLALLARLSGQETVLTGVRLRAGGPLLPLVAETGDDPAATVLLTRAAGALQACAAHAGVPASLLPARHAAAFALADDGPLPADMAIVATDDGACRLELAADVHDAAGADRLAANLAELLQGAAAAPGERLSRLPLLGAAERHRVLVQFNDSAQHFDDTRQLHQMVEDQAAADPGALALLYGSDTMTYEVLNRRANQVAQFLHGHGIGANDRVAVCMERGLEMVVAILGVLKAGAAYMPLDPAYPVERIAYMLDDSAPRALLAQAPLLAALEPVRRLAAELPCLLLAEGLAVLDGLPDANPPAPPLAQAAANLMYVLYTSGSTGRPKGVAMAQGPLVNLIRWQASSRSKLAQRERTLQFSALGFDATFQEIFSALCYGASLVLLAESIRRDPRELVRLMRRYDVERIFLPFVALQNIAEAAVELGEPLPALNTMITAGEQLRISPAIVQFFRMRAGRSLHNYYGPTESHVVTTYVLDGDPGAWPALPPIGAPIANTQIYILDAALQPVALGAHGELYIAGDCLADGYLNRPDLTAERFVGNVFRPGTRMYKTGDIARWLEDGNIEYLGRNDSQVKIRGYRIEPGEVEAALAACAGVREAVVVAREDVPGQKRLVAYLLAQPGHTLAPAALRDRLATVLPDYMVPAAFVCMTAFPVSPNGKLDRRALPAPDAAAQLRQPYEAPQGSTETALAAIWEDLLAVRDVGRRDHFFELGGHSLLAVRLTTRVRQVLQRELALRALFEQPVLADLARVVDGLDSAGTAPLRALPRTPDQVLPLSFAQQRLWFVQELEGPTPTYNMPAALRLTGRLDAAALEPALQYLIERHEVLRTNFDSVEGVPHLRIAPSRTVTLAVTDVAPDEVEARAARHAALPFDLAREPLLRAELLRLSADQHVLLLNVHHIVSDGWSLNILADEWLRAYDALRAGRAPALPVLPLQYADYAYWQREQLTEAVRERQLAYWTGQLAGAPELLDLPTDRVRPAVQRFDGGDEQLRLDPALSHAVRQLGHAHNASLFMTLVTAFGLLLGRLSGQDDVLVGVPQATRDRRELEGMLGMLLGNLVLRMRLDDAAGFGTLLEQVRRTALEAYEHSAIPFEQVVDALPLQRDLSRNPLFQVFFNMLNLPETNYTSPELAIEGLQSTLLDAKFDLTLYAQDSEEGILLHLVYNRGLFDAQRMRELLRQYHSLLEQVSQAPAIACKAVSLLTAPARAVLPDPAVVLDATWHGSIPGRFAALVAAQPAALAVTAAHLQWTYAELDERSEAVACWLQEAGVGAGAVVAICAARRAALVPAVLGVLKAGAAYTIVDPAYPAEHVRACLAVARPAAWLTVAEGGDAALLACLPAPVPRLDLSGNDGWPVLAAGVRAVPAAWTADDVAVLTFTSGSTGLPKAVEGRHGALTHFYPWLQQHFGMGPQDRYALLSGLAHDPLQRDIFNTLWMGASLHVPPVDAIGPGLLADWMAAENISAVNLTPAMLQLLCQDARALPTLRHAFLVGDILTQADVALLQQVAPRCAVVSYYGATEAQRAFGMVEIAPGTAAGLTRDVIALGHGIPGVQLLVLNGAGTLAGIGEVGEVCIRSPHLARGYRDDAAMTARQFVANPFGGGDRLYRTGDLGRYLPDGMVAGLGRNDQQVKLRGFRIELGHVEAALARLPQVREAVVLALGSGEARRLVAYVVPRGTFDADAAAAALRGTLPDYMRPAAYVVLERLPLTPNGKLDRRALPAPAATPAVADTAPATALEASLCALMAELLNRDAVGPAENFFALGGHSLLATRLVSRIRAACGVQLPLRAVFEEPTPAALARLVERAGGDNAGPAPRERSGWHPLSSQQQRLWFLDRFEPANPFYNIPLALRLRGTLVPAQLQQSLDALAARHPSLRTRFATQDGQPVQEILAPAAVPLALTDLTGLAPAQREEAARRAAATVTLQPFVLEQGNLLRAALLRLDDADHVLVLVVHHIVSDGRAGGARRRTRGVYRAGTTGGAAALPPLPLHYSDFAHWQRDWLQQPAALRQLAYWNAQLADAPAVHALPLDRPRPAIQSYRGATHGFAIGAATLAGLRELAAAQAEPTTLFMVLCAAFNVLLYRHSGQADLCIGTPIANRQHDGLDRVVGFFANTLVLRSRPAPGQPFQQFLRDVRATALDAYANQDIAFERVVEAVKPQRHTSHAPLFQVMLSLQESLALPQVDDTLRLEALTLDSSVARFDLTLSLVEEGGTLLAAFEYNTDLFDAATIERWAGHFSHLLDAVVATPQLALDRLPLLDDAERRDVLLASAGERAGPVGDTVLHALFEQQALAHPQRCAAQAGAASITYGELNTRAAELALRLRHAGVAAGDRVAVHAQRSLELLVALLGVLKAGAAYVPLDPAQPQERLAHMLRDSAPAAVLTQQGLAGGALLASVPCRVLLLDGPAAAAPAPLADVLVQPHDLAYVMYTSGSTGMPKGVMVEHASIVNTVRAHVRQCALQAQDRVLQFVSYGFDVSAGEIFGAFAAGATLVLRPDELRVPDEAFAAFLREQAVTVADLPAAFWHQWVHEIAAGRSLPGPALRLVLAGGEKADVARLRTWLTLPATRHVRWINAYGPTETTVNASYMPYDALSEPPAGEVPIGRPIDNTVAYVLDAHLQPVAFGIAGEIYLGGAGVARGYLNQPELTERAFVADPFAGGAARMYRSGDLGRRLDDGTLEYLGRNDSQVKLRGYRIELGEIQSRLATLDGVREACVMLREVAGTPRLVAYLAAAEGMQLSAAELRRMLAASLPDYMVPSAFVWLPVLPVNASGKVETAALPEPGPADMEARVIETPVGAREQLLAQIWQDLLALPQVSRQDHFFELGGHSLMVVTLIDRLHQHDLHVDVRTVFSSPTLAAMAAALADRAGATAAFVAPPNLIPGEFAASASTDQANFEEFEL

SEQ ID NO: 7 - DNA, 6108 bp, Empedobacter Haloabium
atgacattcccacagcttctcgcccacctgcgcagccattccatccacctgaaggccgagcagggcaagctccaggtccgtgccgagaagggcacggtcgatgccgagctgcgcacccagctcgccgcccacaaggaagcgctgctggcgctgctcgccggcgacccggccgcctgtacctggaccgcggcggcgccgcgcatcacgcccgagatgctgccgctggtgcagctgagccagggcgaaatcgatacgatcgttgccgctaccgaaggtggcgcggcggcgatccaggacatctacccgctgtcgccgctgcaggaaggcttcctgttccaccacctgctgcaggccgagggcgacgtctacctggaacgggcgctgatcggcttcgacagccgggacaggctcgatgccttcgtggcggcgctgcagaaggtcatcgaccgccacgacatcctgcgcagcagcgcgcgctggcaggacctgtcgcgccaggtgcaggtggtgcaccggcaggcgcgcctgccggtggtcgaactgaagctgcctgaaggcggcgacggcatggccgtgctgaaggaagcgaccgatccgcgcaagctgcgcctggacctgcaggccgcaccgctgctggcgacacgcatcgtgccggacggcgccagcggcggctggctgatggcgctgctgcatcaccatatggtgtgcgatcacgtgacgctcgaattcatcgtcggcgaggtcgcgctgatcctgggcgggcgcgaggcgctgctgccgccggcactgccgtaccggaacttcatcgcgcagacgctggcggtaccggccagcgcgcacgagggctacttcaagtcccgccttgccgatgtgacggaaaccaccgcgccgttcggcgtgctgaacgtgatgggcgagggcggcgaagtcagcgagggacacgtgcggctcgatggcgcgctggcccagcggatccgcacgcaggcggcgcgcttcggcgtcactaccgccgtcctgttccacgtggcatgggcgcgcgtggttgccctgtgcagcggccgcgacgacgtcgtattcggtaccgtgctgtccggccgcctgcagggcagcgaagccgccgggcgggtgctgggactgttcatcaacgcgctgccgatccgcctcacgctggccggacgcagtaccgaacaactggtgcgcgaaacctacgccgacctgaccgcgttgctggagcacgaacaggcgtcgttgacgctggcacaacaatgcagcggtatcgcggcaccggcgccgctgttcaccagcctgctcaattatcgccacagccacggcggcgcactgcaggccgacggccagtgggacggcatgcgcctgctcgatttcggcgaacgcacgaactatccgatcaccgtttccatcgacgacaccggcgatggctttgaactggaggcgcagtgcgtgaccgggatcgatcccgcgcgcatcgtggactacctggcgaccgccttggccggcctggccgatggcctggcgggcggcaaggccgccaccgagatggcggtgttgccggacgccgaacggacccgcctgctggagctgagccaaggcggcccggcttatggcgcggggctgctgccggccgaactgctggcggcgcgctggccgcaggatgccgccgcgatcgccgtcatcgatggcgagcgccacacgagctatgcggagctggccgcattgagcaaccgcctggcgcagcagatgctggcggccggcgccggacccggcacccgcgtgggcgtcttcgccgagcgcggactggcgatggtcgtggcgctgctcgcggtcgtcaaggcgggcgccacctatctgccgctcgacaccgcgcacccggccgaccgcctcggccacatcctgaacgacagcgcccctgccgccgtgatcctgcaggcagggctggagacggcgctgccgcggcacccggcgaccgccatcgtgctcgatgccgatggcatcgcgcgcggactgccggcggccccggaaagtgcgcccgacctgcgcgcgctgggcgtaacgccggccgacgcggcgtacgtcatctacacttccggttccaccgggctgccgaaaggcgttgccaattcgggcgccggcctggtgaaccgcctggactggttcgccaccgaagtgctggatcacgtgccggtcacggcgatgcgcaccagtatcagcttcgtcgactccgtcaccgaagtcctcgatacgctgctggcgggcggcacgctggtcgtcttcgacaaggccgccacgctcgacccggcgaccttcgcggaaggcacggcgcgctatggcatctcccatctgatggtggtgccggcgctgctgcatcacgtgctggaggtcgcgccgtccgcgctggcacgcgtgcgcaccgtgatcaccagcggcgagcggctgccgccggaactggcgcagcgcctgaaggccgccttcccggccatccggctggtgaacacgtacggctgctccgaagtgaacggtgacgccaccgcctgcgattgcgacggcacggaagcgacggcaacctccgtgatcggccgtccgatcgcgggcgtgcaggcgctggtgctcgatggtgcgcgccagctggtaccgctgggcgctaccggcgagatctacctcggcggcgtgggcgtggcgggcggctacctcaatcgtccggaattgacggccgagcgcttcgtgccgaacccctacggtgcgggcctgctgtacaagacgggcgacctggggcgcctgcgcgccgacggcagcctggaatacctgggccgcaacgacttccaggtcaagatgcgcggcttccgcatcgaactgggcgaaatcgaagcgcggctgcgcacccaccctggcgtcagcgatgccgtcgtggtcgcgcgcacggagcgggccggcgacccgcgcctggccgcgtacgtgctgccgcgccgcgagcgcgccgcggcggccgacgaggccgggttcagcctgttctatttcggtgccacgacctccggagcgggggccgacaaataccggctgtacctggaagcggcccgcttcgccgacgacaacggcttcgaagccatctggacacccgaacgccacttcgacgatgtggctggcctgtatcccaaccctgcgttgctgagcgccgcgctggcgaccagcacgcgccgcgtgcacctgcgcgccggcagcgtggtgctgccgctgcagcagccgatccgggtggtcgaggactggtcggtgctggacaacctgaccggcgggcgggtcggcgtcgcgatcgcctccggctggcacatgcgcgacttcgtgctggcgcccgagcatcacgcgcagcgccaccgcatcatgtacgaaggcatcgagaccgtgcgcgacctgtggcgcggcactgcgcgttcgttccgcgacggcgccggcctgcagagcgaaatccaggtctatccacgcccggtgcaggccgagctgccgatgtggctgacgtcggccggcgccaacgagaccttcatcgaggctggccggctgggactgaacctgctgacccacctgctgggccagaccatccaggaagtggccggcaagatcgccctgtaccgcgaatcgctgcagcggcacggcttcgatccggacagccgcaaggtcacgttgatgatccacacctacgtcggggcggaccaggcggctgccctggcgcaggcacgcgagccgttcaagcgttacatgaaggcgcacgtggggctgctcaaatcgctgtcggccacgctgacgcacgcggtcgacaacgtcgaacaggaaaacctcgacagcctggccgagcacgcgttccagcgttatgcgagcagcgcggccttcatcggctcgcccgagtcgtgcctgccgatctatcggcagttgcgcgaggcgggcgtcgacgaattcgcctgcctgttcgactggatggcgccggaagaagcgctggccggactgccgcagttgcgccggctgcaggacctggcgcgcagcgatgccccgggcgtgcgccagctgcgccgccacctgttggccgcgctgcccgattacatggtgccctcgacgttcagctacttggagcggatgccgctgaccgccagcggcaaggtcaaccgcctggccctgccggcgcccgagcagcaaagtacggaacagacggccttcgatgcgccgcagggcgtcgaggagacctccgtggcacgcctgtggcaggacatgctgaacgttccgccgatcgaccgcaacggcaacttcttcgagttgggcggccactccctgctggccgtgcagatgatcgccgccgtgggcaagctgttcgccacggaggtgccgctgcggcagctgttcgccaatccgaccgtcgccaaattcgccgccgcgattcgcgaacagtcgagcaatgcgaagcatccgaacctggtcacgttgcgcaagcgcggcagcaaggcgccgctgttcctggtgcaccccggcgaaggcgagatcggctacgcgcgcaatctggcaccccatatcgccagcgacgtgccgctgtacggtttcgccgccaccggcctcctgagcggcgaagcgccgttgacgtcgatcgaggagatcgccagccgctacgtgcgcgccatgcgctcggtccagccggaaggtccgtaccgcatcgccggctggtcggccggcggcacgatcgcctacgagatggcccgtcagttgctcggcgtggaccagcaggtcgggttcatcggcctgctcgacaccgacttcagctacgaccacctgtttgcccggaccgatggcgaggaggacctggcgttcgacgagatcaactcgctgctcggttacctgccaccgcggctgccggccgaggtcagcggggaagtgcgcctgctggcgcagagccgcgacttcgatgcgctgctggcgcgcatgcatgcgcacgatttcatcccgaaaggcgtcgatggcggcatcctgcagcgccacctcgccctgcgccatgccctggccgtggcgctgtatcgctatcagccgcagcgcctgccgatcggcgtgacgctgttctcggccagcggcgaaagccgcgtcgacccgacgatcggctggcgcgcgcaccacgcggccgacctgctgcacctgatcccggtcagcggcacgcactatacgatcgtcgaggagccgaacgtcatcgagctgggcaaggccatcagcgcggagctggcccgcagccagccgaacggtccggcaccgtacgcgccgcgcgtcgtcatccagagcggcatggccggcgaggcaccgctgttctgcgtgccgggcgcgggcgccagcgtctcgtcactgcacgaactggcccaggcgctgggcgagaacgtgccggtccatggcctgcaggcgcgcggcctggacggcaccatgctgccgcatgccgacgtgcagtcggccgcgcgggcctatctggccgccgtgcgcgacgtgcagccggccgggccataccggctgctgggccactcgttcggcggctggatcgctttcgagatggcgcagcaactgacggcggccggtgagacggtggagcagctggtcgtcatcgacagccgcagcccggcgccggaaggcacggcggtgcggcactacacccggatcgagacgctgctggaactggtggctctgtacaacctgcgcctggccgacaagctggccctgacggcggccgacttccggccgctcaacccggcggcgcaactggccctgctgcacgagcacctggtgcgcgccggcctggtgtcgccgcgggcccaaccgggcatgctggagggcgtggtgaacgtgctgcaggcgaacctgtcgacggtgtaccggccagccagggtgtatgaaggtgccctgttgctggtcaacgccagcgagcaggaagggcgcggcgacaatgccgcgcgggtggcggcctggcgcagccacgcgccggcgctggtcgaggccgaggcgcctggcaatcacctgacgctgctggcgtcgccgcacgtggacgcggtggccagccgcatcctgggccaggtgccgagcatgctttga

SEQ ID NO: 8 - protein, 2035 aa, Empedobacter Haloabium
MTFPQLLAHLRSHSIHLKAEQGKLQVRAEKGTVDAELRTQLAAHKEALLALLAGDPAACTWTAAAPRITPEMLPLVQLSQGEIDTIVAATEGGAAAIQDIYPLSPLQEGFLFHHLLQAEGDVYLERALIGFDSRDRLDAFVAALQKVIDRHDILRSSARWQDLSRQVQVVHRQARLPVVELKLPEGGDGMAVLKEATDPRKLRLDLQAAPLLATRIVPDGASGGWLMALLHHHMVCDHVTLEFIVGEVALILGGREALLPPALPYRNFIAQTLAVPASAHEGYFKSRLADVTETTAPFGVLNVMGEGGEVSEGHVRLDGALAQRIRTQAARFGVTTAVLFHVAWARVVALCSGRDDVVFGTVLSGRLQGSEAAGRVLGLFINALPIRLTLAGRSTEQLVRETYADLTALLEHEQASLTLAQQCSGIAAPAPLFTSLLNYRHSHGGALQADGQWDGMRLLDFGERTNYPITVSIDDTGDGFELEAQCVTGIDPARIVDYLATALAGLADGLAGGKAATEMAVLPDAERTRLLELSQGGPAYGAGLLPAELLAARWPQDAAAIAVIDGERHTSYAELAALSNRLAQQMLAAGAGPGTRVGVFAERGLAMVVALLAVVKAGATYLPLDTAHPADRLGHILNDSAPAAVILQAGLETALPRHPATAIVLDADGIARGLPAAPESAPDLRALGVTPADAAYVIYTSGSTGLPKGVANSGAGLVNRLDWFATEVLDHVPVTAMRTSISFVDSVTEVLDTLLAGGTLVVFDKAATLDPATFAEGTARYGISHLMVVPALLHHVLEVAPSALARVRTVITSGERLPPELAQRLKAAFPAIRLVNTYGCSEVNGDATACDCDGTEATATSVIGRPIAGVQALVLDGARQLVPLGATGEIYLGGVGVAGGYLNRPELTAERFVPNPYGAGLLYKTGDLGRLRADGSLEYLGRNDFQVKMRGFRIELGEIEARLRTHPGVSDAVVVARTERAGDPRLAAYVLPRRERAAAADEAGFSLFYFGATTSGAGADKYRLYLEAARFADDNGFEAIWTPERHFDDVAGLYPNPALLSAALATSTRRVHLRAGSVVLPLQQPIRVVEDWSVLDNLTGGRVGVAIASGWHMRDFVLAPEHHAQRHRIMYEGIETVRDLWRGTARSFRDGAGLQSEIQVYPRPVQAELPMWLTSAGANETFIEAGRLGLNLLTHLLGQTIQEVAGKIALYRESLQRHGFDPDSRKVTLMIHTYVGADQAAALAQAREPFKRYMKAHVGLLKSLSATLTHAVDNVEQENLDSLAEHAFQRYASSAAFIGSPESCLPIYRQLREAGVDEFACLFDWMAPEEALAGLPQLRRLQDLARSDAPGVRQLRRHLLAALPDYMVPSTFSYLERMPLTASGKVNRLALPAPEQQSTEQTAFDAPQGVEETSVARLWQDMLNVPPIDRNGNFFELGGHSLLAVQMIAAVGKLFATEVPLRQLFANPTVAKFAAAIREQSSNAKHPNLVTLRKRGSKAPLFLVHPGEGEIGYARNLAPHIASDVPLYGFAATGLLSGEAPLTSIEEIASRYVRAMRSVQPEGPYRIAGWSAGGTIAYEMARQLLGVDQQVGFIGLLDTDFSYDHLFARTDGEEDLAFDEINSLLGYLPPRLPAEVSGEVRLLAQSRDFDALLARMHAHDFIPKGVDGGILQRHLALRHALAVALYRYQPQRLPIGVTLFSASGESRVDPTIGWRAHHAADLLHLIPVSGTHYTIVEEPNVIELGKAISAELARSQPNGPAPYAPRVVIQSGMAGEAPLFCVPGAGASVSSLHELAQALGENVPVHGLQARGLDGTMLPHADVQSAARAYLAAVRDVQPAGPYRLLGHSFGGWIAFEMAQQLTAAGETVEQLVVIDSRSPAPEGTAVRHYTRIETLLELVALYNLRLADKLALTAADFRPLNPAAQLALLHEHLVRAGLVSPRAQPGMLEGVVNVLQANLSTVYRPARVYEGALLLVNASEQEGRGDNAARVAAWRSHAPALVEAEAPGNHLTLLASPHVDAVASRILGQVPSML

(19) United States
(12) Patent Application Publication
(10) Pub. No.: US 2009/0124539 A1
(43) Pub. Date: May 14, 2009

(54) Methods of treating infection

(76) Inventors: Ake P. Elhammer, Kalamazoo, MI (US); Torsten Stachelhaus, Kalamazoo, MI (US)

Correspondence Address: HONIGMAN MILLER SCHWARTZ & COHN LLP, 444 WEST MICHIGAN AVENUE, KALAMAZOO, MI, 49007-3714, US

(21) Appl. No.: 12/284,954

(22) Filed: Sep. 26, 2008

Related U.S. Application Data
(63) Continuation-in-part of application No. PCT/US2008/002435, filed on Feb. 25, 2008.
(60) Provisional application No. 60/903,487, filed on Feb. 26, 2007.

(51) Int. Cl. A61K 38/12; C07H 21/00; C07K 14/00; A61P 31/04

(52) U.S. Cl. 514/11; 536/23.1; 530/350

(57) ABSTRACT

The present invention relates to methods of inhibiting the proliferation of bacteria for either ex vivo or in vivo use. The invention also relates to methods of treating a patient infected with an antibiotic resistant bacteria by administering a pharmaceutical composition comprising an Empedopeptin; methods of sanitizing surfaces and instruments; and methods of assaying bacteria for Empedopeptin resistance.

### CROSS-REFERENCE TO RELATED APPLICATIONS

This application is a continuation-in-part of PCT patent application serial no PCT/US2008/002435, filed on Feb. 25, 2008, under 35 U.S.C. § 363, which claims priority to U.S. provisional application Ser. No. 60/903,487, filed Feb. 26, 2007; each of which is hereby incorporated by reference entirely.

### FIELD OF THE INVENTION

The present invention relates to methods of inhibiting the proliferation of bacteria in a patient by administering to the patient an antibiotic compound. The invention also presents ex vivo methods of use for the same antibiotic compound such as methods of sanitizing surfaces and/or objects, and methods of assaying Gram positive bacteria.

### BACKGROUND

Bacteria are unicellular microorganisms. They are typically a few micrometers long and have many shapes including spheres, rods, and spirals. Bacteria are ubiquitous in every habitat on Earth, growing in soil, acidic hot springs, radioactive waste [Fredrickson J, Zachara J, Balkwill D, et al (2004). “Geomicrobiology of high-level nuclear waste-contaminated vadose sediments at the hanford site, Washington state”. Appl Environ Microbiol 70 (7): 4230-41], seawater, and deep in the earth's crust. Some bacteria can even survive in the extreme cold and vacuum of outer space. There are typically 40 million bacterial cells in a gram of soil and a million bacterial cells in a milliliter of fresh water; in all, there are approximately five nonillion (5×10³⁰) bacteria in the world. Whitman W, Coleman D, Wiebe W (1998). “Prokaryotes: the unseen majority”. Proc Natl Acad Sci USA 95 (12): 6578-83. Bacteria are vital in recycling nutrients, and many important steps in nutrient cycles depend on bacteria, such as the fixation of nitrogen from the atmosphere. However, most of these bacteria have not been characterized, and only about half of the phyla of bacteria have species that can be cultured in the laboratory. Rappé M, Giovannoni S. “The uncultured microbial majority”. Annu Rev Microbiol 57: 369-94.

Although the vast majority of these bacteria are rendered harmless or beneficial by the protective effects of the mammalian immune system, a few pathogenic bacteria cause infectious diseases, including cholera, syphilis, anthrax, leprosy and bubonic plague. The most common fatal bacterial diseases are respiratory infections, with tuberculosis alone killing about 2 million people a year, mostly in sub-Saharan Africa. See http://www.who.int/healthinfo/bodgbd2002revised/en/index.html.

Although there are numerous antibiotics that are effective in treating patients suffering from bacterial infections, several recent generations of disease causing bacteria possess multiple drug resistance and have become serious clinical problems.

The number of patients treated for antibiotics-resistant infections has increased drastically in recent years. What started in the 1980s as problem primarily associated with hospital-acquired Enterococcus infections in long-term care patients has become a problem that has moved into the general community and has grown to include a number of common and very serious human pathogens. Drug-resistant Streptococci, Staphylococci and Pseudomonas strains are quite common. In fact, currently as many as 70% of hospital-acquired infections in the US are resistant to at least one antibiotic, and about 40% of S. aureus infections are multidrug-resistant. Coates, A., Hu, Y., Bax, R., and Page, C. (2002) “The Future Challenges Facing the Development of New Antimicrobial Drugs. Nat. Rev. Drug Discov. 1:895-910.

Even very powerful drugs like vancomycin and teicoplanin, which for years represented the “agents of last resort” for treatment of antibiotics-resistant infections, are no longer efficacious against certain strains of bacteria (see e.g., Smith, T. L., and Jarvis, W. R. (1999) Antimicrobial resistance in Staphylococcus aureus. Microb. Infect. 1:795-805; Ge, M., Chen, Z., Onishi, H. R., Kohler, J., Silver, L. L., Kerns, R., Fuzukawa, S., Thompson, C., and Kahne, D. (1999) Vancomycin derivatives that inhibit peptidoglycan biosynthesis without binding D-Ala-D-Ala. Science 284:507-511; and Goldman, R. C., and Gange, D. (2000) Inhibition of transglycosylation involved in bacterial peptidoglycan synthesis. Curr. Med. Chem. 7:801-820). Hence, these compounds are predicted to be of little use for the treatment of future infections. In this context, it is important to realize that the loss of efficacy of vancomycin and related compounds leaves very few treatment options for patients with multi-drug resistant infections. The seriousness of the situation is clearly illustrated by the fact that as many as 90,000, of the two million people who acquired a bacterial infection in US hospitals in 2004, died as a result of it (Leeb, M. (2004) A shot in the arm. Nature 431:892-893). There is clearly an immediate need for new antibiotics with novel modes of action. Thus, there is a strong demand for a compound having excellent antibacterial activity against antibiotic resistant strains of disease causing bacteria.

### SUMMARY OF THE INVENTION

The present invention provides methods of inhibiting bacterial proliferation including providing a pharmaceutical composition comprising Empedopeptin or a pharmaceutically acceptable salt thereof, wherein the bacteria comprises at least one Gram positive strain.

In several embodiments, the Gram positive strain is resistant to glycopeptides, aminoglycosides, oxazolidinones, penicillins, macrolides, rifamycins, polypeptides, lipopeptides, chloramphenicol, or any combination thereof. For example, the Gram positive strain further comprises Enterococcus faecalis, Staphylococcus aureus, Staphylococcus epidermidis, Streptococcus pneumoniae, Streptococcus pyogenes, or any combination thereof.

In other embodiments, the Gram positive strain is resistant to at least one of linezolid, oxacillin, vancomycin, daptomycin, erythromycin, methicillin, gentamicin, chloramphenicol, fusidic acid, rifampin, or combinations thereof. For instance, the Gram positive strain is resistant to methicillin.

In several embodiments, the Gram positive strain consists essentially of Enterococcus faecalis, the Gram positive strain consists essentially of Staphylococcus aureus, the Gram positive strain consists essentially of Staphylococcus epidermidis, the Gram positive strain consists essentially of Streptococcus pneumoniae, or the Gram positive strain consists essentially of Streptococcus pyogenes.

In some embodiments, the method further includes providing a second antibiotic agent. For instance, some methods further include providing a second pharmaceutical composition, wherein the second pharmaceutical composition comprises a second antibiotic agent, or providing a single pharmaceutical composition comprising Empedopeptin and a second antibiotic agent.

Another aspect of the present invention provides methods of treating a patient infected with bacteria including providing a pharmaceutical composition comprising Empedopeptin or a pharmaceutically acceptable salt thereof, wherein the bacteria comprises at least one Gram positive strain.

In several embodiments, the Gram positive strain is resistant to one or more of glycopeptides, oxazolidinones, penicillins, macrolides, rifamycins, polypeptides, lipopeptides, chloramphenicol, or combinations thereof. For example, the Gram positive strain further comprises Enterococcus faecalis, Staphylococcus aureus, Staphylococcus epidermidis, Streptococcus pneumoniae, Streptococcus pyogenes, or any combination thereof. For example, the Gram positive strain is resistant to linezolid, oxacillin, vancomycin, daptomycin, methicillin, gentamicin, erythromycin, chloramphenicol, fusidic acid, rifampin, or any combination thereof. In other examples, the Gram positive strain is resistant to methicillin.

In several embodiments, the Gram positive strain consists essentially of Enterococcus faecalis, the Gram positive strain consists essentially of Staphylococcus aureus, the Gram positive strain consists essentially of Staphylococcus epidermidis, the Gram positive strain consists essentially of Streptococcus pneumoniae, or the Gram positive strain consists essentially of Streptococcus pyogenes.

In some embodiments, the method further includes providing a second antibiotic agent. For instance, some methods further include providing a second pharmaceutical composition, wherein the second pharmaceutical composition comprises a second antibiotic agent, or providing a single pharmaceutical composition comprising Empedopeptin and a second antibiotic agent.

Another aspect of the present invention provides methods of treating a patient infected with Staphylococcus aureus or Staphylococcus epidermidis, either of which is resistant to glycopeptides, oxazolidinones, penicillins, macrolides, rifamycins, polypeptides, lipopeptides, chloramphenicol, or any combination thereof, comprising administering to the patient an effective amount of a pharmaceutical composition comprising Empedopeptin or a pharmaceutically acceptable salt thereof.

In several embodiments, the pharmaceutical composition is administered to the patient parenterally or intravenously. In other embodiments, the pharmaceutical composition is intravenously administered to the patient, or the pharmaceutical composition is topically administered to the patient.

Another aspect of the present invention provides methods of sanitizing a surface or object comprising contacting the surface or object with a cleaning composition comprising Empedopeptin and a carrier.

In several embodiments, the carrier comprises water or alcohol.

In other embodiments, the surface is skin, or the object is an agricultural product, a medical instrument, a kitchen utensil, or an article of clothing.

In some embodiments, the cleaning composition further comprises a second antibiotic agent, e.g., one that does not substantially affect the antibiotic activity of Empedobactin.

Another aspect of the present invention provides methods of assaying bacteria for Empedopeptin resistance comprising colonizing bacteria in a medium; and incubating the medium, wherein the medium comprises Empedopeptin.

Another aspect of the present invention provides an isolated nucleotide sequence comprising SEQ. ID. NO. 1.

Another aspect of the present invention provides an isolated protein sequence comprising SEQ. ID. NO. 2.

Another aspect of the present invention provides an isolated nucleotide sequence comprising SEQ. ID. NO. 3.

Another aspect of the present invention provides an isolated protein sequence comprising SEQ. ID. NO. 4.

Another aspect of the present invention provides an isolated nucleotide sequence comprising SEQ. ID. NO. 5.

Another aspect of the present invention provides an isolated protein sequence comprising SEQ. ID. NO. 6.

Another aspect of the present invention provides an isolated nucleotide sequence comprising SEQ. ID. NO. 7

Another aspect of the present invention provides an isolated protein sequence comprising SEQ. ID. NO. 8.

### BRIEF DESCRIPTION OF THE FIGURES

FIG. 1 illustrates a gene cluster sequence that is responsible for the biosynthesis of Empedopeptin in E. haloabium;

FIG. 2 illustrates an organization of the Empedopeptin biosynthesis gene;

FIG. 3 provides the sequence listing for SEQ ID NO 1

### DETAILED DESCRIPTION

The present invention provides methods of restricting bacterial proliferation by providing a pharmaceutical composition comprising Empedopeptin, wherein the bacteria comprises at least one Gram positive strain that is resistant to one or more of aminoglycosides, carbacephems, carbapenems, cephalosporins (e.g., first generation, second generation, third generation, or fourth generation), glycopeptides, lipopeptides, macrolides, monobactams, penicillins, polypeptides, quinolones, sulfonamides, tetracyclines, oxazolidinones, rifamycins, other unclassified antibiotics (e.g., chloramphenicol), or combinations thereof. This method is useful for ex vivo or in vivo purposes.

### I. DEFINITIONS

As used herein, “Empedopeptin”, refers to a cyclic peptide having the structure:

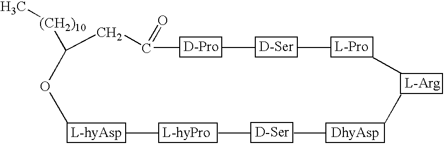

As used herein, “antibiotic” or “antibiotic agent” refers to a compound, such as penicillin, streptomycin, methicillin, vancomycin, erythromycin, daptomycin, and/or bacitracin produced by or derived from certain fungi, bacteria, and other organisms, or are synthetically produced, that can destroy or inhibit the growth of other microorganisms. Antibiotics are widely used in the prevention and treatment of infectious diseases such as bacterial infection. Common antibiotics are discussed below.

As used herein, “antibiotic resistant” or “antibiotic resistance” refers to a characteristic of some bacteria, wherein at least some portion of a population of bacteria can survive and proliferate despite being treated with large amounts of antibiotic. For example, antibiotic resistance is used to mean that the bacteria does not lyse or is not otherwise destroyed by the antibiotic. Antibiotic resistance can also mean that the bacteria actively grows and proliferates in the presence of the antibiotic. In several examples, antibiotic resistant bacteria are those that when treated with one or more antibiotics yield a minimal inhibitory concentration from between about 2-fold to more than about 100-fold higher (e.g., from about 3 fold to about more than 100 fold, from about 4 fold to about more than 100 fold, or the like) than that observed for bacteria sensitive to the one or more antibiotic(s), or bacteria having intermediate resistance to the one or more antibiotic(s).

As used herein, “alcohol” refers to an organic compound in any physical state (e.g., solid, gas, or liquid) that includes a carbon atom that is bonded to a hydroxy (—OH) functional group. Without limitation, exemplary alcohols include methanol, ethanol, propanol, isopropanol, or the like.

As used herein, “bacteria” means ubiquitous one-celled organisms, spherical, spiral, or rod-shaped and appearing singly or in chains, comprising the Schizomycota, a phylum of the kingdom Monera (in some classification systems the plant class Schizomycetes), various species of which are involved in fermentation, putrefaction, infectious diseases, or nitrogen fixation.

As used herein, “bacterial proliferation” means growth or reproduction of bacteria.

As used herein, “an effective amount” is defined as the amount required to confer a therapeutic effect on the treated patient, and is typically determined based on age, surface area, weight, and condition of the patient. The interrelationship of dosages for animals and humans (based on milliGrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970).

As used herein, “agricultural product” means fruits, vegetables, nuts, flowers, honey, and animal products such as beef, pork, chicken, fish, lamb, or the like.

As used herein, “medical instrument” means instruments associated with medical uses such as a scalpels, hemostats, saws, retractors, forceps, surgical needles, catheters, drills, bandages, rib spreaders, tongue depressors, and any other instrument that is commonly inserted into a living organism.

As used herein, “kitchen utensils” means instruments commonly used in food preparation such as knives, forks, spoons, tongs, spatulas, any other instruments that are commonly used in food preparation.

As used herein, “Gram positive” refers to bacteria that retain a crystal violet color during the Gram stain process. Gram positive bacteria will appear blue or violet under a microscope.

As used herein, “Gram negative” refers to bacteria that retain a red or pink color during the Gram stain process. Gram negative bacteria will appear red or pink under a microscope. The difference in classification between Gram positive and Gram negative bacteria is largely based on a difference in the bacteria's cell wall structure.

As used herein, “patient” refers to a mammal, including a human.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays, or as therapeutic agents.

### II. ABBREVIATIONS

Abbreviations used herein have the following meanings:

L-Arg: L-Arginine

D-Ser: D-Serine

L-Pro: L-Proline

D-Pro: D-Proline

L-Ala: L-Alanine

L-Thr: L-Threonine

D-aThr: D-allo-Threonine

L-hyPro: L-trans-3-hydroxyproline

D-hyAsp: D-threo-β-hydroxyaspartic acid

L-hyAsp: L-threo-β-hydroxyaspartic acid

### III. METHODS

The present invention provides methods of inhibiting bacterial proliferation comprising providing a pharmaceutical composition comprising Empedopeptin or a pharmaceutically acceptable salt thereof, wherein the bacteria comprises at least one Gram positive strain, and the Gram positive strain is resistant to one or more of glycopeptides, lipopeptides, aminoglycosides, oxazolidinones, penicillins, macrolides, rifamycins, polypeptides, other unclassified antibiotics (e.g., chloramphenicol), or combinations thereof. In several methods, the Gram positive strain further comprises Enterococcus faecalis, Staphylococcus aureus, Staphylococcus epidermidis, Streptococcus pneumoniae, Streptococcus pyogenes, or any combination thereof.

For example, in one group of methods, the Gram positive strain comprises Enterococcus faecalis, Staphylococcus aureus, Staphylococcus epidermidis, Streptococcus pneumoniae, Streptococcus pyogenes, or any combination thereof; and the Gram positive strain is further resistant to one or more glycopeptides including amikacin, gentamicin, kanamycin, neomycin, netilmicin, paromomycin, streptomycin, tobramycin, vancomcin, teicoplanin, and apramycin. In other methods, the Gram positive strain comprises Enterococcus faecalis, Staphylococcus aureus, Staphylococcus epidermidis, Streptococcus pneumoniae, Streptococcus pyogenes, or any combination thereof; and the Gram positive strain is further resistant to one or more penicillins including methicillin, dicloxacillin, flucloxacillin, oxacillin, nafcillin, amoxicillin, ampicillin, azlocillin, carbenicillin, cloxacillin, mezlocillin, penicillin, piperacillin, ticarcillin, or any combination thereof. In another method, the Gram positive strain comprises Enterococcus faecalis, Staphylococcus aureus, Staphylococcus epidermidis, Streptococcus pneumoniae, Streptococcus pyogenes, or any combination thereof; and the Gram positive strain is further resistant to one or more aminoglycosides including amikacin, gentamicin, kanamycin, neomycin, netilmicin, paromomycin, streptomycin, tobramycin, apramycin, or combinations thereof. In another method, the Gram positive strain comprises Enterococcus faecalis, Staphylococcus aureus, Staphylococcus epidermidis, Streptococcus pneumoniae, Streptococcus pyogenes, or combinations thereof; and the Gram positive strain is further resistant to one or more macrolides including erythromycin, azithromycin, troleandomycin, clarithromycin, dirithromycin, roxithromycin, or any combination thereof. In another method, the Gram positive strain comprises Enterococcus faecalis, Staphylococcus aureus, Staphylococcus epidermidis, Streptococcus pneumoniae, Streptococcus pyogenes, or any combination thereof; and the Gram positive strain is further resistant to one or more rifamycins including rifampin, rifabutin, rifapentine, or any combination thereof. In another method, the Gram positive strain comprises Enterococcus faecalis, Staphylococcus aureus, Staphylococcus epidermidis, Streptococcus pneumoniae, Streptococcus pyogenes, or any combination thereof; and the Gram positive strain is further resistant to one or more polypeptides or lipopeptides including daptomycin, bacitracin, colistin, polymyxin B, or any combination thereof. In other methods, the Gram positive strain comprises Enterococcus faecalis, Staphylococcus aureus, Staphylococcus epidermidis, Streptococcus pneumoniae, Streptococcus pyogenes, or any combination thereof; and the Gram positive strain is further resistant to one or more of linezolid, oxacillin, vancomycin, daptomycin, methicillin, gentamicin, chloramphenicol, fusidic acid, rifampin, or any combination thereof.

In several alternative methods, the Gram positive strain consists essentially of Enterococcus faecalis that is resistant to one or more of linezolid, oxacillin, vancomycin, daptomycin, methicillin, gentamicin, chloramphenicol, fusidic acid, rifampin, or combinations thereof. In other methods, the Gram positive strain consists essentially of Staphylococcus aureus that is resistant to one or more of linezolid, oxacillin, vancomycin, daptomycin, methicillin, gentamicin, chloramphenicol, fusidic acid, rifampin, or combinations thereof. In several methods, the Gram positive strain consists essentially of Staphylococcus epidermidis that is resistant to one or more of linezolid, oxacillin, vancomycin, daptomycin, methicillin, gentamicin, chloramphenicol, fusidic acid, rifampin, or combinations thereof. In other methods, the Gram positive strain consists essentially of Streptococcus pneumoniae that is resistant to one or more of linezolid, oxacillin, vancomycin, daptomycin, methicillin, gentamicin, chloramphenicol, fusidic acid, rifampin, or combinations thereof. In other methods, the Gram positive strain consists essentially of Streptococcus pyogenes that is resistant to one or more of linezolid, oxacillin, vancomycin, daptomycin, methicillin, gentamicin, chloramphenicol, fusidic acid, rifampin, or combinations thereof.

The methods of inhibiting bacterial proliferation are also useful for treating a patient infected with bacteria, wherein the bacteria is a Gram positive strain that is resistant to glycopeptides, lipopeptides, aminoglycosides, oxazolidinones, penicillins, macrolides, rifamycins, polypeptides, or other unclassified antibiotics (e.g., chloramphenicol), or any combination thereof.

Such methods comprise providing a pharmaceutical composition comprising Empedopeptin or a pharmaceutically acceptable salt thereof to treat an infection of Gram positive bacteria that are resistant to glycopeptides, lipopeptides, aminoglycosides, oxazolidinones, penicillins, macrolides, rifamycins, polypeptides, unclassified antibiotics (e.g., chloramphenicol), or combinations thereof.

In several methods, a patient infected with bacteria is treated with a pharmaceutical composition comprising Empedopeptin or a pharmaceutically acceptable salt thereof, wherein the bacteria comprises at least one Gram positive strain, and the Gram positive strain is resistant to one or more glycopeptides, lipopeptides, aminoglycosides, oxazolidinones, penicillins, macrolides, rifamycins, polypeptides, unclassified antibiotics (e.g., chloramphenicol), or combinations thereof. In other methods, patient is infected with Enterococcus faecalis that is resistant to glycopeptides, aminoglycosides, oxazolidinones, lipopeptides, penicillins, macrolides, rifamycins, polypeptides, unclassified antibiotics (e.g., chloramphenicol), or combinations thereof. In several methods, the patient is infected with Staphylococcus aureus that is resistant to one or more glycopeptides, aminoglycosides, lipopeptides, oxazolidinones, penicillins, macrolides, rifamycins, polypeptides, unclassified antibiotics (e.g., chloramphenicol), or combinations thereof. In several methods, the patient is infected with Staphylococcus epidermidis that is resistant to one or more glycopeptides, lipopeptides, aminoglycosides, oxazolidinones, penicillins, macrolides, rifamycins, polypeptides, unclassified antibiotics (e.g., chloramphenicol), or combinations thereof. In other methods, the patient is infected with Streptococcus pneumoniae that is resistant to one or more glycopeptides, lipopeptides, aminoglycosides, oxazolidinones, penicillins, macrolides, rifamycins, polypeptides, or unclassified antibiotics (e.g., chloramphenicol), or combinations thereof. In some methods, the patient is infected with Streptococcus pyogenes that is resistant to one or more of linezolid, oxacillin, vancomycin, daptomycin, methicillin, gentamicin, chloramphenicol, fusidic acid, rifampin, or combinations thereof.

Other methods provide for treating a patient infected with bacteria comprising providing Empedopeptin, or a pharmaceutically acceptable salt thereof, wherein the bacteria comprises Enterococcus faecalis, Staphylococcus aureus, Staphylococcus epidermidis, Streptococcus pneumoniae, Streptococcus pyogenes, or combinations thereof. More specifically, the bacteria comprises methicillin resistant Staphylococcus aureus, methicillin resistant Streptococcus pneumoniae, methicillin resistant Streptococcus pyogenes, or combinations thereof. In several methods, the population of bacteria is resistant to linezolid, oxacillin, vancomycin, daptomycin, methicillin, gentamicin, chloramphenicol, fusidic acid, rifampin, or combinations thereof. In other embodiments, the population of bacteria consists essentially of Enterococcus faecalis. In still more embodiments, the population of bacteria consists essentially of Staphylococcus aureus. Alternatively, the population of bacteria consists essentially of Staphylococcus epidermidis. Or, the population of bacteria consists essentially of Streptococcus pneumoniae. In some embodiments, the population of bacteria consists essentially of Streptococcus pyogenes.

Other embodiments of the present invention provide methods of treating a patient infected with Staphylococcus aureus or Staphylococcus epidermidis, either of which is resistant to linezolid, oxacillin, vancomycin, daptomycin, methicillin, gentamicin, chloramphenicol, fusidic acid, rifampin, or combinations thereof, comprising administering to the patient an effective amount of Empedopeptin or a pharmaceutically acceptable salt thereof.

Still more embodiments provide methods of sanitizing a surface or object comprising contacting the surface or object with a cleaning composition comprising Empedopeptin and an effective carrier. Several cleaning compositions of the present invention include a carrier comprising water, alcohol, or mixtures thereof. In other examples, the solvent comprises ethanol, methanol, isopropanol, water, or combinations thereof. This method is well-suited for sanitizing surfaces such as skin, countertops, tabletops, and other surfaces that can host infectious bacteria. Moreover, this method is well-suited for sanitizing objects such as surgical instruments (e.g., scalpel, oral thermometer, retractor, saw blades, forceps, hemostat, scissors, or the like), kitchen utensils, or the like.

In several embodiments, the pharmaceutical composition useful for treating infection or restricting the proliferation of bacteria can optionally include a second antibiotic agent. For instance the pharmaceutical composition can comprise Empedopeptin and one or more antibiotic agents independently selected from glycopeptides, lipopeptides, aminoglycosides, oxazolidinones, penicillins, macrolides, rifamycins, polypeptides, or unclassified antibiotics (e.g., chloramphenicol).

Another aspect of the present invention provides methods of assaying bacteria for Empedopeptin resistance comprising colonizing bacteria in a medium comprising Empedopetin, and incubating the bacteria. Any bacteria can be assayed using this method.

### IV. ANTIBIOTICS

Antibiotics are often classified by the scope of their respective bioactivities. An antibiotic's scope of bioactivity is qualitatively assessed as being narrow spectrum, moderate spectrum, or broad spectrum.

Narrow spectrum antibiotics have activity in only a few strains of bacteria or small family of bacteria, while antibiotics having activities in multiple strains or families of bacteria are classified as moderate spectrum antibiotics, and those antibiotics having activities in a large number of strains or families of bacteria (e.g., Gram negative bacteria and/or Gram positive bacteria) are classifies as broad spectrum antibiotics.

Antibiotics can also be classified by the organisms against which they are effective, and by the type of infection in which they are useful, which depends on the sensitivities of the organisms that most commonly cause the infection and the concentration of antibiotic obtainable in the affected tissue.

At the most generic level, antibiotics can be classified as either bactericidal or bacteriostatic. Bactericidals kill bacteria directly where bacteriostatics prevent them from dividing. However, these classifications are based on laboratory behavior; in practice, both of these can end a bacterial infection.

Common commercial antibiotics include aminoglycosides, carbacephems, carbapenems, cephalosporins (e.g., first generation, second generation, third generation, or fourth generation), glycopeptides, lipopeptides, macrolides, monobactams, penicillins, polypeptides, quinolones, sulfonamides, tetracyclines, oxazolidinones, rifamycins, and unclassified antibiotics (e.g., chloramphenicol). Each class of antibiotic is briefly discussed below.

Penicillins include those antibiotic drugs obtained from penicillium molds or produced synthetically, which are most active against Gram-positive bacteria and used in the treatment of various infections and diseases. Penicillin is one of the beta-lactam antibiotics, all of which possess a four-ring beta-lactam structure fused with a five-membered thiazolidine ring. These antibiotics are nontoxic and kill sensitive bacteria during their growth stage by the inhibition of biosynthesis of their cell wall mucopeptide. Penicillin antibiotics provide narrow spectrum bioactivity, moderate or intermediate spectrum bioactivity, and broad spectrum bioactivity. Without limitation, narrow spectrum penicillins include methicillin, dicloxacillin, flucloxacillin, oxacillin, nafcillin, or the like. Without limitation, moderate or intermediate spectrum penicillins include amoxicillin, ampicillin, or the like. Penicillins include, without limitation, ampicillin, azlocillin, carbenicillin, cloxacillin, dicloxacillin, flucloxacillin, mezlocillin, nafcillin, penicillin, piperacillin, and ticarcillin.

Aminoglycosides are a group of antibiotics that are effective against certain types of bacteria. They include amikacin, gentamicin, kanamycin, neomycin, netilmicin, paromomycin, streptomycin, tobramycin and apramycin. Those which are derived from Streptomyces genus are named with the suffix -mycin, while those which are derived from micromonospora are named with the suffix -micin. Aminoglycosides are useful primarily in infections involving aerobic, Gram-negative bacteria, such as Pseudomonas, Acinetobacter, and Enterobacter. In addition, some mycobacteria, including the bacteria that cause tuberculosis, are susceptible to aminoglycosides. The most frequent use of aminoglycosides is empiric therapy for serious infections such as septicemia, complicated intraabdominal infections, complicated urinary tract infections, and nosocomial respiratory tract infections. Usually, once cultures of the causal organism are grown and their susceptibilities tested, aminoglycosides are discontinued in favor of less toxic antibiotics.

Carbacephem is a class of antibiotic medication, specifically modified forms of cephalosporin. It prevents bacterial cell division by inhibiting cell wall synthesis. Without limitation, carbacephems include loracarbef, or the like.

Carbapenems are a class of beta-lactam antibiotics, the structure of which renders them highly resistant to beta-lactamases. Carbapenems include, without limitation, imipenem (often given as part of imipenem/cilastatin), meropenem, ertapenem, faropenem, doripenem, panipenem/betamipron, or the like.

Cephalosporins are a class of beta-lactam antibiotics. Together with cephamycins they belong to a sub-group called cephems. First-generation cephalosporins are predominantly active against Gram positive bacteria. First generation cephalosporins are moderate spectrum agents, with a spectrum of activity that includes penicillinase-producing, methicillin-susceptible staphylococci and streptococci, though they are not the drugs of choice for such infections. They also have activity against some Escherichia coli, Klebsiella pneumoniae and Proteus mirabilis, but have no activity against Bacteroides fragilis, enterococci, methicillin-resistant staphylococci, Pseudomonas, Acinetobacter, Enterobacter, indole-positive Proteus or Serratia. First generation cephalosporins include, without limitation, cefadroxil, cefazolin, and cephalexin.

The second generation cephalosporins have a greater Gram negative spectrum while retaining some activity against Gram positive cocci. They are also more resistant to beta-lactamase. Second generation cephalosporins include, for example, cefonicid, cefprozil, cefproxil, cefuroxime, cefuzonam, cefaclor, cefamandole, ceforanide, and cefotiam.

Third generation cephalosporins have a broad spectrum of activity and further increased activity against Gram negative organisms. Some members of this group (particularly those available in an oral formulation, and those with anti-pseudomonal activity) have decreased activity against Gram positive organisms. They may be particularly useful in treating hospital-acquired infections, although increasing levels of extended-spectrum beta-lactamases are reducing the clinical utility of this class of antibiotics. Without limitation, third generation cephalosporins include cefcapene, cefdaloxime, cefdinir, cefditoren, cefetamet, cefixime, cefmenoxime, cefodizime, cefoperazone, cefotaxime, cefpimizole, cefpodoxime, cefteram, ceftibuten, ceftiofur, ceftiolene, ceftizoxime, and ceftriaxone. Third generation cephalosporins with antipseudomonal activity include ceftazidime, cefpiramide, and cefsulodin.

Oxacephems are also sometimes grouped with third-generation cephalosporins and include latamoxef and flomoxef.

Fourth generation cephalosporins are extended-spectrum agents with similar activity against Gram positive organisms as first-generation cephalosporins. They also have a greater resistance to beta-lactamases than the third generation cephalosporins. Many can cross blood brain barrier and are effective in meningitis. Exemplary fourth generation cephalosporins include cefclidine, cefepime, cefluprenam, cefoselis, cefozopran, cefpirome, and cefquinome.

These cephems have progressed far enough to be named, but have not been assigned to a particular generation: ceftobiprole, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, ceftioxide, ceftobiprole, ceftobiprole, and cefuracetime.

Glycopeptide antibiotics are another class of antibiotic drugs. They consist of a glycosylated cyclic or polycyclic nonribosomal peptide. Exemplary glycopeptide antibiotics include vancomycin, teicoplanin, ramoplanin, and decaplanin.

Macrolides are a group of drugs (typically antibiotics) whose activity stems from the presence of a macrolide ring, a large lactone ring to which one or more deoxy sugars, usually cladinose and desosamine, are attached. The lactone ring can be either 14-, 15- or 16-membered. Macrolides belong to the polyketide class of natural products. Common antibiotic macrolides include erythromycin, azithromycin, troleandomycin, clarithromycin, dirithromycin, and roxithromycin.

Monobactams are beta-lactam antibiotics wherein the beta-lactam ring is alone, and not fused to another ring (in contrast to most other beta-lactams, which have at least two rings). An example is aztreonam.

Polypeptide antibiotics include bacitracin, colistin, and polymyxin B.

Quinolones are another family of broad spectrum antibiotics. The parent of the group is nalidixic acid. The majority of quinolones in clinical use belong to the subset of fluoroquinolones, which have a fluoro group attached the central ring system. Exemplary quinolone antibiotics include cinoxacin, flumequine, nalidixic acid, oxolinic acid, piromidic acid, pipemidic acid, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin mesilate, sparfloxacin, temafloxacin, tosufloxacin, clinafloxacin, gemifloxacin, moxifloxacin, gatifloxacin, sitafloxacin, and trovafloxacin.

Antibacterial sulfonamides (sometimes called simply sulfa drugs) are synthetic antimicrobial agents that contain the sulfonamide group. In bacteria, antibacterial sulfonamides act as competitive inhibitors of the enzyme dihydropteroate synthetase, DHPS. Several antibacterial sulfonamides include mafenide prontosil, sulfacetamide, sulfamethizole, sulfanilamide, sulfasalazine, sulfisoxazole, trimethoprim, and trimethoprim-sulfamethoxazole.

Tetracyclines are a group of broad-spectrum antibiotics named for their four (“tetra-”) hydrocarbon rings (“-cycl-”) derivation (“-ine”). Exemplary tetracyclines include tetracycline, chlortetracycline, oxytetracycline, demeclocycline, doxycycline, lymecycline, meclocycline, methacycline, minocycline, rolitetracycline, and tigecycline.

Oxazolidinones are a class of compounds containing 2-oxazolidone in their structures. Oxazolidinones are useful antibiotics. Some of the most important oxazolidinones are the last generation of antibiotics used against Gram positive bacterial strains. One example of an oxazolidinone is linezolid.

Rifamycins are a group antibiotics that are synthesized either naturally by the bacterium Amycolatopsis mediterranei, or artificially. Rifamycins are particularly effective against mycobacteria, and are therefore used to treat tuberculosis, leprosy, and mycobacterium avium complex (MAC) infections. The rifamycin antibiotic group includes, without limitation, rifampin, rifL.

Lipopeptide antibiotics includes peptides with attached lipids or a mixture of lipids and peptides such as the cyclic lipopeptide, daptomycin.

Other unclassified antibiotics include chloramphenicol, clindamycin, ethambutol, fosfomycin, furazolidone, isoniazid, metronidazole, mupirocin, nitrofurantoin, platensimycin, pyrazinamide, quinupristin/dalfopristin, spectinomycin, and telithromycin.

Pharmaceutical compositions comprising the abovementioned antibiotics can comprise a combination of antibiotics.

Furthermore, the abovementioned antibiotics can be administered via any suitable method (e.g., orally, topically, intravenously, ip injection, muscular injection (IM), or by any combination thereof). These antibiotics can further be administered concurrently, i.e., at approximately the same time, or sequentially, i.e., at different times.

Recent generations of bacteria have developed resistance to one or more of the abovementioned antibiotic agents. Such bacteria include Enterococcus faecalis, Staphylococcus aureus, Staphylococcus epidermidis, Streptococcus pneumoniae, Streptococcus pyogenes, each of which can cause illness in mammals; especially humans.

### V. PRODUCING EMPEDOPEPTIN

Cyclic peptides are composed of several biosynthetic units, typically amino acids, linked in sequence to form a closed ring structure. The producing organisms contain large enzyme complexes referred to as non-ribosomal peptide synthetase (NRPS) complexes, which are responsible for the synthesis of these molecules. NRPS complexes have an assembly line-like organization comprising a number of biosynthetic modules, each of which is responsible for the addition of one, specific amino acid (biosynthetic unit) to the sequence of the cyclic peptide.

Because each biosynthetic module in the NRPS complex is specific for a certain amino acid, the sequential arrangement of the modules in the complex does, in itself, determine the sequence and structure of the cyclic peptide produced. From this follows that if the sequence, or order, of the modules is changed, the amino acid sequence of the peptide will also change. That is, if a biosynthetic module specific for a particular amino acid is substituted for a module specific for another amino acid, the net effect will be a different amino acid, at that position, in the peptide produced by the modified NRPS complex. Moreover, since the arrangement of modules in an NRPS complex is a direct reflection of the arrangement of the module-encoding gene sequences in the corresponding NRPS gene, deletion, insertion and/or substitution of biosynthetic modules in an NRPS complex can be accomplished by deletion, insertion and/or substitution of the relevant sequence segments in the corresponding NRPS gene. Consequently, genetic engineering (of the relevant cyclic peptide-producing organism) can now be used to generate molecules with features that previously could only be introduced using the complicated and expensive synthetic chemistry methods discussed above.

Nonetheless, utilization of the genetic engineering approach outlined above, for introduction of modifications to the structure of Empedopeptin, requires knowledge of the sequence and structure of the NRPS gene encoding the Empedopeptin synthetase. This gene has to date not been identified or cloned. Consequently, with the aim of cloning this gene, a set of degenerate PCR primers, targeted at the coding regions of the highly conserved core adenylation domain sequence motifs A3 (AUG1470: GGWTCYACWGGWACWCCWTTRCC; forward) and A8 (AUG1473: CCWARYTCWATACGRAAWCCACG; reverse; with R=A or G; W=A or T; Y═C or T), were prepared. The design of the primers was optimized with regard to the codon usage of the Empedopeptin-producer organism Empedobacter spp. ATCC 31962. A PCR amplification was subsequently carried out using these primers, standard reaction conditions and the Expand High-Fidelity PCR system (Roche), according to the manufacturer's protocol. The reaction yielded an 806 bp DNA fragment, which was cloned and subjected to sequence analysis. This revealed that the fragment encodes a portion of an NRPS adenylation domain. The amplified fragment shares highest amino acid sequence homology (55% identity, 66% similarity) with the proline-activating adenylation domain of module 2 in the syringopeptin synthetase from Pseudomonas syringae pv. syringae. Determination and analysis of the presumed substrate-binding constituents, in the fragment sequence, revealed that the adenylation domain amplified from Empedobacter spp. likely recognizes and activates proline. Together these observations suggest that the cloned PCR fragment represents a fragment of the Empedopeptin synthetase NRPS gene.

The sequence of the putative Empedopeptin synthase fragment is SEQ ID NO 1, and is provided in the sequence listing below.

The corresponding protein sequence is SEQ. ID. NO. 2, and is also provided in the sequence listing below.

The first step in the cloning of the remaining portion(s) the Empedopeptin synthetase NRPS gene (epp) cluster involved construction of an Empedobacter haloabium fosmid library. This was done using the CopyControl Cloning System (Epicentre) which combines the clone stability afforded by single copy cloning with the advantages of high yields of DNA obtained by on-demand induction of clones to a high copy number (usually 10-200 copies per cell). First, high-molecular-weight E. haloabium genomic DNA (>80 kb) was prepared, using standard procedures. The genomic DNA was then sheared to approximately 40 kb fragments which, subsequently, were end-repaired to generate the appropriate blunt and 5′-phosphorylated ends. The end-repaired DNA was then size-fractioned on a low-melting-point agarose gel, using field-inversion gel electrophoresis (FIGE). DNA fragments of the appropriate size (approx. 40 kb) were excised, extracted from the gel, and, subsequently, ligated into the CopyControl pCC1FOS cloning vector. Following packaging of the ligated DNA into Lambda phage particles, the packaging reaction mix was used for transfection of Escherichia coli EPI300-T1, to determine the library's titer. And, once the titer was determined the library was plated and screened.

Individual clones derived from plating of the fosmid library were screened by PCR, using primers designed to amplify the NRPS gene fragment, previously amplified from E. haloabium genomic DNA (see above). E. haloabium belongs to the family of Flavobacteriaceae (e.g. Flavobacterium johnsoniae, Flavobacterium pschrophilum, and Flavobacterium sp. MED217), which has an average genome size of approximately 4.4 Mb. Consequently, about 500 clones were screened to ensure a 99% probability of finding at least one clone that contained the (entire) sequence information of the (putative) empedopeptin biosynthetic gene cluster (predicted size: approx. 30 kb).

Twelve 48-well-microtiter plates were prepared by adding 0.8 ml of Luria-Broth (LB) medium, supplemented with 12.5 μg/ml chloramphenicol, and inoculating the medium in each well with a single clone from the plated fosmid library (see above). Following overnight incubation at 30° C./250 rpm, 20 μl of each culture was used as inoculum for the copy number amplification procedure outlined below. The remainder of the cultures were supplemented with 0.4 ml glycerol and stored, as a master plate, at −80° C. The aliquots induced for copy number amplification produced the (high) yields of fosmid DNA required for PCR analysis and fingerprinting. Fresh 48-well-microtiter plates were prepared by adding 0.8 ml LB medium, supplemented with 12.5 μg/ml chloramphenicol and 0.1% arabinose, and inoculating the medium in each well with 20 μl of the pre-culture prepared earlier. The cultures were incubated overnight at 30° C./250 rpm. To reduce the time and effort involved in the screening of the fosmid clones, small aliquots of the individual cultures were combined into defined pools (of 24 clones each), and the (fosmid) DNA present in each pool was isolated using standard procedures. The pooled fosmid DNAs was used as template in PCR amplifications with primers designed to amplify the NRPS gene fragment isolated previously by degenerate primer PCR (see above). Genomic E. haloabium DNA and/or the previously cloned putative empedopeptin NRPS gene fragment was used as positive controls for these experiments. Fosmid DNA from the individual clones in the clone pools that produced an amplicon of the expected size (in the first round of PCR) were subsequently prepared and analyzed individually in the same manner. This second round of PCR identified two individual fosmid clone(s) that, upon sequencing, were found to both contain the entire NRPS portion of the (putative) empedopeptin biosynthetic gene cluster.

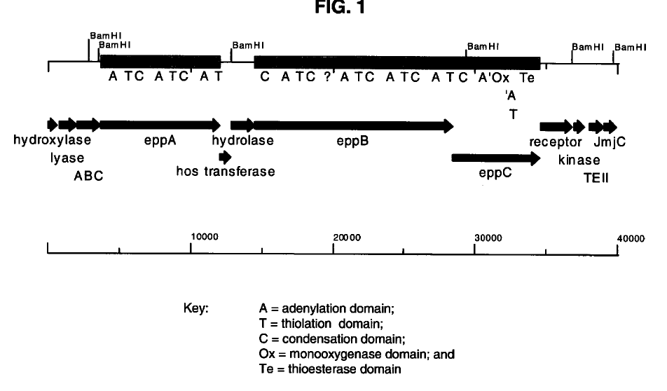

An illustration of the gene cluster sequence identified in two fosmid clones prepared from E. haloabium genomic DNA is provided as FIG. 1. The locations of sequences encoding putative “decorating enzymes” are also indicated in FIG. 1.

In FIG. 1, the following abbreviations are employed: A, adenylation domain; T, thiolation domain; C, condensation domain; Ox, monooxygenase domain; and Te, thioesterase domain.

The isolated nucleotide and protein sequences of the three NRPS genes comprising the Empedopeptin biosynthetic gene cluster are also provided as follows:

| (Nucleotide Sequence of Empedopeptin synthase fragment) | |
|---|---|
| SEQ ID NO 1 | |
| gtcgggttcg acgggtacgc caaaggggtc gcgatggccc agggcccgct ggtcaacctg | 60 |
| atccggtggc aggcttcgtc gcgttcgaag ctggcccagc gcgaacgcac gctgcagttc | 120 |
| tccgccctgg gcttcgatgc cacgttccag gagatcttca gcgcattgtg ctatggcgcc | 180 |
| agcctggtgc tgctggccga gtccatccgg cgcgatccgc gcgaactggt gcggctgatg | 240 |
| cgccggtacg acgtggaacg cattttcctg ccgttcgtcg cgctgcagaa catcgccgag | 300 |
| gcggcggtgg agctgggcga accgttgcct gcgctgaaca cgatgatcac ggcaggcgaa | 360 |
| cagttgcgca tcagtcccgc catcgtgcag ttcttccgca tgcgcgccgg ccgcagcctg | 420 |
| cacaactact acggcccgac cgagagccac gtcgtgacga cgtatgtgct ggacggcgat | 480 |
| ccgggcgcgt ggcccgcgtt gccgccgatc ggcgcgccga tcgccaacac ccagatctac | 540 |
| attctcgacg cggcgctgca gccggtggcc ctgggcgcgc atggcgagct gtatatcgcc | 600 |
| ggcgattgcc tggccgacgg ctacctgaac cggcctgacc tgacggcgga gcgcttcgtc | 660 |
| ggcaatgtct tccggccagg cacgcgcatg tacaagacgg gcgacatcgc ccgctggctg | 720 |
| gaggacggca atatcgaata cctgggccgc aacgacagcc aggtcaagat ccgcggctac | 780 |
| cgcatcgagc tgggcgaaat cgaggc | 806 |
| (Peptide Sequence of Empedopeptin synthase fragment) | |
| SEQ ID NO 2 | |
| Val Gly Phe Asp Gly Tyr Ala Lys Gly Val Ala Met Ala Gln Gly Pro | |
| 1 5 10 15 | |
| Leu Val Asn Leu Ile Arg Trp Gln Ala Ser Ser Arg Ser Lys Leu Ala | |
| 20 25 30 | |
| Gln Arg Glu Arg Thr Leu Gln Phe Ser Ala Leu Gly Phe Asp Ala Thr | |
| 35 40 45 | |
| Phe Gln Glu Ile Phe Ser Ala Leu Cys Tyr Gly Ala Ser Leu Val Leu | |
| 50 55 60 | |
| Leu Ala Glu Ser Ile Arg Arg Asp Pro Arg Glu Leu Val Arg Leu Met | |
| 65 70 75 80 | |
| Arg Arg Tyr Asp Val Glu Arg Ile Phe Leu Pro Phe Val Ala Leu Gln | |
| 85 90 95 | |
| Asn Ile Ala Glu Ala Ala Val Glu Leu Gly Glu Pro Leu Pro Ala Leu | |
| 100 105 110 | |
| Asn Thr Met Ile Thr Ala Gly Glu Gln Leu Arg Ile Ser Pro Ala Ile | |
| 115 120 125 | |
| Val Gln Phe Phe Arg Met Arg Ala Gly Arg Ser Leu His Asn Tyr Tyr | |
| 130 135 140 | |
| Gly Pro Thr Glu Ser His Val Val Thr Thr Tyr Val Leu Asp Gly Asp | |
| 145 150 155 160 | |
| Pro Gly Ala Trp Pro Ala Leu Pro Pro Ile Gly Ala Pro Ile Ala Asn | |
| 165 170 175 | |
| Thr Gln Ile Tyr Ile Leu Asp Ala Ala Leu Gln Pro Val Ala Leu Gly | |
| 180 185 190 | |
| Ala His Gly Glu Leu Tyr Ile Ala Gly Asp Cys Leu Ala Asp Gly Tyr | |
| 195 200 205 | |
| Leu Asn Arg Pro Asp Leu Thr Ala Glu Arg Phe Val Gly Asn Val Phe | |
| 210 215 220 | |
| Arg Pro Gly Thr Arg Met Tyr Lys Thr Gly Asp Ile Ala Arg Trp Leu | |
| 225 230 235 240 | |
| Glu Asp Gly Asn Ile Glu Tyr Leu Gly Arg Asn Asp Ser Gln Val Lys | |
| 245 250 255 | |
| Ile Arg Gly Tyr Arg Ile Glu Leu Gly Glu Ile Glu | |
| 260 265 | |
| (Nucleotide Sequence of eppA) | |
| SEQ ID NO 3 | |
| atgcatacct ccgccatacc cgacacctgc gcgaccttgt tcgacgtcct ccgccatcgt | 60 |
| gccagcgccg ccggcacggc ggaccggccg gccttcacct atctgaacga tggtgaatcg | 120 |
| gtcagcggtg cgctcagtta tgcccagctc gacgccgcgg cgcagcgcct ggcggcgcac | 180 |
| ctgcagcagg tcaccagccc gggcgaccgc gtgctgctcg tgtatccgcc cagcctggac | 240 |
| tacatcgtcg ccttctatgc ctgcgtgtac gccggtgtca ccgccgtgcc cgcgctgccg | 300 |
| ccggccaatc cgcgtgccct gccgcggctg cggctgcagg cggaagacgc ccagcccagc | 360 |
| gcggccctga ccagcgccgc gatccgcgcc acgatcgtcg atggcgcggc gggcgacgac | 420 |
| gcgctgcgcc gctgccactg gctggcgacc gatgcgctgg acgagacggc gccgccatgg | 480 |
| cgcgagccgt cggtgcgtgc cagcgacatc gtgttcctgc agtacacctc gggttcgacc | 540 |
| ggtgcgccca aaggcgtcat ggtgagccat gccagcctgc tggccaacgt cgccctcagc | 600 |
| cagcagctgt acggcatgcg cggcgacgac gtgttcgtct cgtggctgcc gccgcaccac | 660 |
| gacttcggcc tgatcggcac gatcgtctcg ccggtctatg tcggctgcca cagcgtgcag | 720 |
| ttcccgcccg ccgcgttcct gatgcgcccg caccgctggc tcaagctcat cgcggcatac | 780 |
| cgcgcccgca tcaccggcgc gcccaacttc gcctaccagt tgtgcgcgca gcgcgtcacg | 840 |
| ccggcgcagc gtgccggcct cgatctgtcc tgcctcgagg tcgcggtcaa cggcgccgag | 900 |
| cgtatccgca tggagacggt acgggagttc gccgccgcct tcgccgactg cggcctgagg | 960 |
| ccggaagcga tggtgccggc gtatggcatg gccgagtgtg tgctgctggc ttgcgcggcg | 1020 |
| atggacaagc ggccgggcgc cttgccgcac agccgccatc tcagcaaggc ggcgctggag | 1080 |
| cgcaacgtcg tgaccgacag cgccggcgcg gcggacgaga tcgagattgc ctgcacgggc | 1140 |
| gcggccgtca acggcgcgca ccgcatcgtt tgcgtcgagc cggacagccg cgtggcgctg | 1200 |
| ccggacaacg cggtcggcga agtctggatc agcggcccat ccgtcgccga tggctactgg | 1260 |
| ggcaagccgg acgccagcgc ggcggtattc ggcgccgcgc tggccggtgg ccccggccgc | 1320 |
| tggttgcgca cgggcgacct gggattcgtc gccgatggcc gcctgtacat cacgggccgc | 1380 |
| atcaaggaaa tgatgatctt taacggccgc aacgtctatc cgcaggacgt cgagatcacg | 1440 |
| gtcgagaagc tcgataccgc tttccggccc agcggctgcg ccgtgttcgc ggtggaggac | 1500 |
| gacgccacga ccgcgctggt cgtcgtgcag gagctcgagg cgcgccagca ggcctacacg | 1560 |
| gccacgctgg tggcccgact gcgcgaggcg ctggccgagc gccacgacat cctcgacctg | 1620 |
| gccggtgtcg tgctggtcaa ggcgggccgc attccacgca cctccagcgg caagctgcag | 1680 |
| cgcgtggcgt gccgccagct gtatctggaa ggcgccctcg atcccatctg gagctggcgc | 1740 |
| cgtgaagacg acagcgtggc cgcggtggcg ggtgccgtcg cacccgccga gcagcgcatg | 1800 |
| ctggcgatct ggcaggagct gttcgagcag gcgccgctgg cgctggacga caatttcttc | 1860 |
| cgcctgggcg gccactcgct gctggcgacc cagctgatcg gtgccgtcaa cgcggcattc | 1920 |
| ggcgtgcagc tgccgctgcg ggtcgtgttc cacgcgccga ccccgcgggc gatggccgcg | 1980 |
| gcggtcggtg acgcggccgc gggcggcgcc accgatgtgc tggcgccggc cgggcacgcg | 2040 |
| ggtctggcgc cgctgtcgtt cgcccagcag cgtttctggt tcctcgacca gtaccagccc | 2100 |
| ggcaacccgt tctacaacat cccgctggcg ctcgcgctga cgggcgccgt cgatgccgca | 2160 |
| ctgctggaac gggcgctgaa cgcgctggtc gcgcggcatg acacgctgcg taccagcttc | 2220 |
| cccgccgacg gcggcgtgcc gcggcagcac gtggcggcgc agctggcgct gccgctgacc | 2280 |
| atcgtcgacc tggccgcgct gccggtcgcc gaggccgagg cgcgcaccga acgcatcgtg | 2340 |
| cgtgccgagg ccgcgcagcc attcgacctg acggccggtc cattgctgcg agccagtctg | 2400 |
| gtgtcgattg ccgatacgcg ccatgtgctg ctgctgacgt tgcaccacat cgtgcacgac | 2460 |
| ggctggtcca cgccggtgct gctgggcgaa ctgcgccgca tctacgcggc gctgcgcgac | 2520 |
| agtcaggccg cggccctgcc tgcgccggcc ttgcagtacg ccgactatgc cgtgtgggag | 2580 |
| cagcgccgct ggcagggcga ggcgctggcc gcggcgctgg cattctggcg cgccaacctg | 2640 |
| gccgacgcct cgccgctgct ggcgctgccg accgaccggc cgcgcgccaa tgtgatggcg | 2700 |
| cacgaaggcc gggcatggca gacgcgcgtg ccggcggcac tggtgcgcga cctgaaccgg | 2760 |
| cttgccgcca gctcgaacgc gacgctgttc atggtgctga ccgcggcgtt gaacgccgtg | 2820 |
| ctgtaccgct attccggcca gaccgatttc gccatcggcg ccctgtcggc caaccgcccg | 2880 |
| gcaggtaccg agcacatgcc gggcaacttc gtcaacgtgg tgccgctgcg tgcccgcgtg | 2940 |
| cacggcgacg atacgttcgc ggcgctgctt gccgatacgg cggcgaacct gctggccgcc | 3000 |
| tacgactgcc agctgccgtt cgagttgatc ctgcagcacg tggtgtccga gcgcagcccg | 3060 |
| gcctacacgc cctatgcgca ggtggtactg aattaccaca gcgagttcga aggccaggaa | 3120 |
| caggcggcgc tggcaccgga cggcgacgcg ctccacatcg aaggccgcca cgcggccagc | 3180 |
| gtccagtacg cggcgttcga cctgaagatc gagatgaacc gcgtcggcgc cgagctggac | 3240 |
| ctggtgttcg agtacagcac ggcgctgttc gaccaagcga cgatcgcccg gctggccggc | 3300 |
| cactacgtgc gcgtgctcga acaggtcggc gccgatgccc aggcgcgtgt cgccgcgctg | 3360 |
| gcgctgctgt cggaaggtga gctggcggcg ctgtcggcgc agtggcagtc cgcccgccac | 3420 |
| gattacccgc gcacggccaa cctggccacg ctgctggagc agcaggccgc gcgcacgccg | 3480 |
| gatgcgccgg cggtggcttg cgccggcacg gtgctgacgt acgcccagtt gcacggccgg | 3540 |
| gccaaccgcc tggcccacct gctgcgcgcg cgcggcgtcg ggccggacgt gctggtgggc | 3600 |
| gtctgcgtcg agcgttcgct cgacatggtc gtggccgtgc tggccgtcgt caaggccggc | 3660 |
| ggtgcctacc tgccgctcga cccgaactat ccggccgcgc gcctcgcata catgctggaa | 3720 |
| gacgccgccc cggcgctggt gctgacgcaa cagcacctgg ccgcgcgcct gccggcgcag | 3780 |
| gcgccggcca tcgtgatcga cgccgatcac acggcacacc cggacagcgc accggctccg | 3840 |
| gtgggcgggc cggacgacct ggcatacgtc atctacacgt ccggttcgac cggcaagccg | 3900 |
| aagggcgcca tggtgcagcg ccagggcgtg ctgaacctgc tgacgtggtt cgtgcgcgag | 3960 |
| tacgccatcg gcgcggccga tcgcgtgctg ctggtgtcgt ccttcagctt cgacctgacg | 4020 |
| cagaagaaca tcttcggcat cctgctggtc ggcggcgagc tgcacctgat ggcggacgac | 4080 |
| tacgcgccgg aacgcatcgg cgcctatgcg gggaccgccg ggatcacgct gatcaactgc | 4140 |
| gcgcccagcg cgttctatcc gctgctggcc gacggcggcg cggcgcgcat ggcgtcgctg | 4200 |
| cgcgccgtct tcctgggcgg cgagccgatc caggtcggcc tgctgcgcgc ggcataccgc | 4260 |
| gacgtcgcca cgccaccact ggtgcacaac acgtacggcc cgaccgaggc ctccgatgtc | 4320 |
| gtgtcgcact acgcctggca cccgcatgag ccggtgacga cgctgccgat cggccgggcg | 4380 |
| atcgccaaca cccgcctgta tgtgctcgat ggcggccgcc agctggtgcc gcaaggcgcc | 4440 |
| gtgggcgagc tgtatgtggg cggcgacggg gtcgggcgcg gctatctgca ccgtcccgaa | 4500 |
| ctgaccgccg aacggttcct gcccgatccg tttgccgggc agccaggcgc gcgcatgtac | 4560 |
| cgcaccggcg acctggtgcg ctccctgccg gacggcgtgc tggaatacct gggccgtatc | 4620 |
| gatcaccagg tcaaggtgcg tggcctgcgc atcgagctcg gggaaatcga agaggcgctg | 4680 |
| gcggcgctgc cggccatcga ccaggcactg gtgctcgcct gcgacgatct ggccgccgat | 4740 |
| gtgcgcctgg tcgcctacct ggtcggcgtc gatgcgcagg ccgcgctcga tcccgtcgca | 4800 |
| ctgcgtgcgg cgctgacgca aaccctgccg cagtacatgc tgccgtcgca tttcgtccag | 4860 |
| ctgccggcgt tcccgttgag ccccaacggc aaggtggacc gggccgcgtt gccgcgaccc | 4920 |
| gtacaggacc tgcatgcacc gttcgtcgcg ccgagcggcg ccaccgagca ggcgctcgcg | 4980 |
| caaatctggg cggaggtgct gaagtgtgcc gacgtgggtc gcgccgacga cttcttccag | 5040 |
| ctgggcggcc actcgctgct ggccacgcag gtgatgtcgc atgtgcgcgc gcgccttggc | 5100 |
| gtcgacctgc cgctgcgcac cctgttcgaa tacccgacgc tggcggcact gggcgagcag | 5160 |
| atcgaccgcg ccgacaaggc cgcgagcggc ccgctggccc tggccgccgg cgacggcgcc | 5220 |
| gcggcgggcg cgttggcgcc gctgtcgtat gcgcagcagc gcctgtgggt gctgcagaag | 5280 |
| ctgggcgaga atccggccgt ctacaacctg ccgttcgccg tcgagctcga gggggcggtc | 5340 |
| gacgtgcccg cgttgcagca cgcgctggac ctgctggcgc ggcggcacgc ggcgttgcgt | 5400 |
| accgccttcg tcaccgtcga cggcgagccg ctgtgcgcgg tggccgccca tgccgcgttg | 5460 |
| ccgctgcaga ccgccagcct ggccgacgcg gcgccgcagg cggtgcacga ctggctggtc | 5520 |
| gccgcggcgc aggtgccgtt cgacctggag tgcgcgccac tggcgcgtgc gaccctgttg | 5580 |
| cacgtcgcgc cggcccggca cgtgctgctg ctggtcatgc accacatcat tgccgacggc | 5640 |
| tggtcgatcg gtgtcctgag ccgcgaactg tcggtgctgt acaacgccgc ccgccgtggt | 5700 |
| gtgccggcgg cactgccggc cttgccgctc cagtacagcg attatgcccg ctggcagcgc | 5760 |
| agccgcgcgg aagagggcgc gttcgacaat cagctggctt actggcgcga ccgcctggcg | 5820 |
| cacgcgcccg ccatgctggc cttgccgctg gaccatccgc gtccggccct gccggccctg | 5880 |
| cgcggcgacg tgctggcttt caccgtcgaa ccgggcctgc tggcaggcct gcggcgcctg | 5940 |
| gcgcgcgaag ggcaggcgag cctgttcatg gtactgagcg ccgccttcgg tgtgctgctg | 6000 |
| ggccgctact ccggccagcg cgacctgtgc atcggcacgc cgatcgccaa ccgccatcat | 6060 |
| ggcgagctgg aaggattggt cggcttcttc gtcaacacgc tcgtgctgcg cctgacgctc | 6120 |
| gagccggcgc acggcttcga ggcgctgctg gcgcaggtgc gcgaaacggt gctgcaggca | 6180 |
| ttcgccaacc aggacctccc gttcgaacag gtcgtggcgg ccagcgccgg tgcgcgccag | 6240 |
| gccggccaga cgccgctgtt ccaggccatg ctcgcgctgc agaacgcgcc gcaggacgag | 6300 |
| gtggcgctgg aggccctgtc cggccgcgtg ctcgacgtgc acaacggtgg cgccaaattc | 6360 |
| gacctgacgc tcgacatcac gccgcgcggc gaccgcctgg actgccgctt tgaatacgat | 6420 |
| tgcgcgctgt tcgaacgcgc cacggtggcg cgcttggccg ataacctgct cacactgctg | 6480 |
| gccagcatcg tcgccgcgcc gcaggcaccg ttgcaaacgc tggcattgct ggcgccagcc | 6540 |
| gagcaggcct tgctggcacg gctgggcgcc ggcacgcccg ccggcgccgc gccgctggtg | 6600 |
| catcgcgcat tcgagtccca cgcggcacgc aacccggacg ccgtggcatt gacgcacgaa | 6660 |
| ggtaccaccc tgacgtacgc cgaattgaac gcgcgggccg acacgctggc acgcgcgctt | 6720 |
| acggccgccg gggtgggacc ggacagccgg gtggtcctgt atgccgaacg cggcatcgga | 6780 |
| ttgatcaccg gtgtgctggc gatcctgaaa gctggcggcg cctacgtgcc attcgatccg | 6840 |
| gcgtatccgc gcgaacggct ggcatacatg gcacaggact gcatgccggc ggcgctcgtc | 6900 |
| acggaaccgg cgctgctggc cgaggcacag gcgctgggac cggccctggc ggccgtgccc | 6960 |
| tgctgcctga tcgaagcggg cggcgcgcag cccggcgctg cgccggcgcc ggcatcgggc | 7020 |
| gccgccgttg gccccggcca tctcgcttac atgatctata cctccggctc gacgggacag | 7080 |
| ccgaaaggcg tgcaggtgga acatggcggc ctggccagcc tggcggcgga ccagaaccgg | 7140 |
| gcgctggcga tcggtcccgg cagccgcgtg ctgcaattcg cgtcgatcag cttcgatgcc | 7200 |
| agcatctggg aaatcgtcat ggcgctggcc agcggcgcgg cgctggtttc cgcaccgcgc | 7260 |
| gccgcgctga tgccgggcgc gccgctgctc gcgttcctgg gcgagcagaa catcagccac | 7320 |
| gcgctgctgc caccttcggt gctggcgatc atggctgacg acgagcggct ggcgccgatg | 7380 |
| acgttgctgg tgggcggcga agcctgcccg ccgtccgtcg ccgcccactg gggccggcgc | 7440 |
| caccgtttcg tcaacgccta tggtccgagc gagatcacgg tctgcgccac gacctggcat | 7500 |
| tacgacggcc gcgccggcgg cgccattccg atcgggcggc cactggcggg tacccgcatc | 7560 |
| catatcctgg acgaggcggg ccagccggta ccggtcggcg cggtcggcga gatccatatc | 7620 |
| ggcggcgtcg gcgtggcgcg cggttacctg aaccggccgg acctgaccgc acagcgcttc | 7680 |
| ctggccgaac cggggcaccc cgatacccgc ttgtaccgca ccggcgacct ggggcgatgg | 7740 |
| gatgcggccg gcatgctgca ctatgcgggc cgcaacgatt tccaggtcaa ggtacggggc | 7800 |
| ttccgcatcg agctgggcga aatcgaagcc gtgctgcgcg cccagccggc attggccgat | 7860 |
| gccgccgtga tcgcccgtgc gggggcggac ggccagcagc gcctgctggc ctatgtggtg | 7920 |
| ccacgcgcgg atacggcgcc cgaaccggcg gccctgcgca gcgccttgct ggcacgcctg | 7980 |
| ccggactaca tggtgcctgg agcgttcatc gcgctgccgg cattgccgca gacacccaac | 8040 |
| ggcaagctcg atcgcgatgc gctgccgctg cccgatgacg atgccttggc gcggcaggct | 8100 |
| ttcgtgccgc cgcaggacgg catcgagcgg cgcctggccg acatctggca aggcgtgctc | 8160 |
| ggtgtcgcgg cggtgggccg tttcgatcac ttcttcgagc tgggcggcca ctcgctggcg | 8220 |
| ttgacgaagc tcagcttcct ggtgcaggaa gcgttcggcg tgacgctcag cctgggtcag | 8280 |
| ctctaccagc tgcagcagct ggcgcagcag gccgaccata tcgccgcggc gcttgccacg | 8340 |
| gcaagccgca agaaggtgct ggtactggac ctggacgacg aggaggaagc cgcatga | 8397 |
| (Protein Sequence of eppA) | |
| SEQ ID NO 4 | |
| Met His Thr Ser Ala Ile Pro Asp Thr Cys Ala Thr Leu Phe Asp Val | |
| 1 5 10 15 | |
| Leu Arg His Arg Ala Ser Ala Ala Gly Thr Ala Asp Arg Pro Ala Phe | |
| 20 25 30 | |
| Thr Tyr Leu Asn Asp Gly Glu Ser Val Ser Gly Ala Leu Ser Tyr Ala | |
| 35 40 45 | |
| Gln Leu Asp Ala Ala Ala Gln Arg Leu Ala Ala His Leu Gln Gln Val | |
| 50 55 60 | |
| Thr Ser Pro Gly Asp Arg Val Leu Leu Val Tyr Pro Pro Ser Leu Asp | |
| 65 70 75 80 | |
| Tyr Ile Val Ala Phe Tyr Ala Cys Val Tyr Ala Gly Val Thr Ala Val | |
| 85 90 95 | |
| Pro Ala Leu Pro Pro Ala Asn Pro Arg Ala Leu Pro Arg Leu Arg Leu | |
| 100 105 110 | |
| Gln Ala Glu Asp Ala Gln Pro Ser Ala Ala Leu Thr Ser Ala Ala Ile | |
| 115 120 125 | |
| Arg Ala Thr Ile Val Asp Gly Ala Ala Gly Asp Asp Ala Leu Arg Arg | |
| 130 135 140 | |
| Cys His Trp Leu Ala Thr Asp Ala Leu Asp Glu Thr Ala Pro Pro Trp | |
| 145 150 155 160 | |
| Arg Glu Pro Ser Val Arg Ala Ser Asp Ile Val Phe Leu Gln Tyr Thr | |
| 165 170 175 | |
| Ser Gly Ser Thr Gly Ala Pro Lys Gly Val Met Val Ser His Ala Ser | |
| 180 185 190 | |
| Leu Leu Ala Asn Val Ala Leu Ser Gln Gln Leu Tyr Gly Met Arg Gly | |
| 195 200 205 | |
| Asp Asp Val Phe Val Ser Trp Leu Pro Pro His His Asp Phe Gly Leu | |
| 210 215 220 | |
| Ile Gly Thr Ile Val Ser Pro Val Tyr Val Gly Cys His Ser Val Gln | |
| 225 230 235 240 | |
| Phe Pro Pro Ala Ala Phe Leu Met Arg Pro His Arg Trp Leu Lys Leu | |
| 245 250 255 | |
| Ile Ala Ala Tyr Arg Ala Arg Ile Thr Gly Ala Pro Asn Phe Ala Tyr | |
| 260 265 270 | |
| Gln Leu Cys Ala Gln Arg Val Thr Pro Ala Gln Arg Ala Gly Leu Asp | |
| 275 280 285 | |
| Leu Ser Cys Leu Glu Val Ala Val Asn Gly Ala Glu Arg Ile Arg Met | |
| 290 295 300 | |
| Glu Thr Val Arg Glu Phe Ala Ala Ala Phe Ala Asp Cys Gly Leu Arg | |
| 305 310 315 320 | |
| Pro Glu Ala Met Val Pro Ala Tyr Gly Met Ala Glu Cys Val Leu Leu | |
| 325 330 335 | |
| Ala Cys Ala Ala Met Asp Lys Arg Pro Gly Ala Leu Pro His Ser Arg | |
| 340 345 350 | |
| His Leu Ser Lys Ala Ala Leu Glu Arg Asn Val Val Thr Asp Ser Ala | |
| 355 360 365 | |
| Gly Ala Ala Asp Glu Ile Glu Ile Ala Cys Thr Gly Ala Ala Val Asn | |
| 370 375 380 | |
| Gly Ala His Arg Ile Val Cys Val Glu Pro Asp Ser Arg Val Ala Leu | |
| 385 390 395 400 | |
| Pro Asp Asn Ala Val Gly Glu Val Trp Ile Ser Gly Pro Ser Val Ala | |
| 405 410 415 | |
| Asp Gly Tyr Trp Gly Lys Pro Asp Ala Ser Ala Ala Val Phe Gly Ala | |
| 420 425 430 | |
| Ala Leu Ala Gly Gly Pro Gly Arg Trp Leu Arg Thr Gly Asp Leu Gly | |
| 435 440 445 | |
| Phe Val Ala Asp Gly Arg Leu Tyr Ile Thr Gly Arg Ile Lys Glu Met | |
| 450 455 460 | |
| Met Ile Phe Asn Gly Arg Asn Val Tyr Pro Gln Asp Val Glu Ile Thr | |
| 465 470 475 480 | |
| Val Glu Lys Leu Asp Thr Ala Phe Arg Pro Ser Gly Cys Ala Val Phe | |
| 485 490 495 | |
| Ala Val Glu Asp Asp Ala Thr Thr Ala Leu Val Val Val Gln Glu Leu | |
| 500 505 510 | |
| Glu Ala Arg Gln Gln Ala Tyr Thr Ala Thr Leu Val Ala Arg Leu Arg | |
| 515 520 525 | |
| Glu Ala Leu Ala Glu Arg His Asp Ile Leu Asp Leu Ala Gly Val Val | |
| 530 535 540 | |
| Leu Val Lys Ala Gly Arg Ile Pro Arg Thr Ser Ser Gly Lys Leu Gln | |
| 545 550 555 560 | |
| Arg Val Ala Cys Arg Gln Leu Tyr Leu Glu Gly Ala Leu Asp Pro Ile | |
| 565 570 575 | |
| Trp Ser Trp Arg Arg Glu Asp Asp Ser Val Ala Ala Val Ala Gly Ala | |
| 580 585 590 | |
| Val Ala Pro Ala Glu Gln Arg Met Leu Ala Ile Trp Gln Glu Leu Phe | |
| 595 600 605 | |
| Glu Gln Ala Pro Leu Ala Leu Asp Asp Asn Phe Phe Arg Leu Gly Gly | |
| 610 615 620 | |
| His Ser Leu Leu Ala Thr Gln Leu Ile Gly Ala Val Asn Ala Ala Phe | |
| 625 630 635 640 | |
| Gly Val Gln Leu Pro Leu Arg Val Val Phe His Ala Pro Thr Pro Arg | |
| 645 650 655 | |
| Ala Met Ala Ala Ala Val Gly Asp Ala Ala Ala Gly Gly Ala Thr Asp | |
| 660 665 670 | |
| Val Leu Ala Pro Ala Gly His Ala Gly Leu Ala Pro Leu Ser Phe Ala | |
| 675 680 685 | |
| Gln Gln Arg Phe Trp Phe Leu Asp Gln Tyr Gln Pro Gly Asn Pro Phe | |
| 690 695 700 | |
| Tyr Asn Ile Pro Leu Ala Leu Ala Leu Thr Gly Ala Val Asp Ala Ala | |
| 705 710 715 720 | |
| Leu Leu Glu Arg Ala Leu Asn Ala Leu Val Ala Arg His Asp Thr Leu | |
| 725 730 735 | |
| Arg Thr Ser Phe Pro Ala Asp Gly Gly Val Pro Arg Gln His Val Ala | |
| 740 745 750 | |
| Ala Gln Leu Ala Leu Pro Leu Thr Ile Val Asp Leu Ala Ala Leu Pro | |
| 755 760 765 | |
| Val Ala Glu Ala Glu Ala Arg Thr Glu Arg Ile Val Arg Ala Glu Ala | |
| 770 775 780 | |
| Ala Gln Pro Phe Asp Leu Thr Ala Gly Pro Leu Leu Arg Ala Ser Leu | |
| 785 790 795 800 | |
| Val Ser Ile Ala Asp Thr Arg His Val Leu Leu Leu Thr Leu His His | |
| 805 810 815 | |
| Ile Val His Asp Gly Trp Ser Thr Pro Val Leu Leu Gly Glu Leu Arg | |
| 820 825 830 | |
| Arg Ile Tyr Ala Ala Leu Arg Asp Ser Gln Ala Ala Ala Leu Pro Ala | |
| 835 840 845 | |
| Pro Ala Leu Gln Tyr Ala Asp Tyr Ala Val Trp Glu Gln Arg Arg Trp | |
| 850 855 860 | |
| Gln Gly Glu Ala Leu Ala Ala Ala Leu Ala Phe Trp Arg Ala Asn Leu | |
| 865 870 875 880 | |
| Ala Asp Ala Ser Pro Leu Leu Ala Leu Pro Thr Asp Arg Pro Arg Ala | |
| 885 890 895 | |
| Asn Val Met Ala His Glu Gly Arg Ala Trp Gln Thr Arg Val Pro Ala | |
| 900 905 910 | |
| Ala Leu Val Arg Asp Leu Asn Arg Leu Ala Ala Ser Ser Asn Ala Thr | |
| 915 920 925 | |
| Leu Phe Met Val Leu Thr Ala Ala Leu Asn Ala Val Leu Tyr Arg Tyr | |
| 930 935 940 | |
| Ser Gly Gln Thr Asp Phe Ala Ile Gly Ala Leu Ser Ala Asn Arg Pro | |
| 945 950 955 960 | |
| Ala Gly Thr Glu His Met Pro Gly Asn Phe Val Asn Val Val Pro Leu | |
| 965 970 975 | |
| Arg Ala Arg Val His Gly Asp Asp Thr Phe Ala Ala Leu Leu Ala Asp | |
| 980 985 990 | |
| Thr Ala Ala Asn Leu Leu Ala Ala Tyr Asp Cys Gln Leu Pro Phe Glu | |
| 995 1000 1005 | |
| Leu Ile Leu Gln His Val Val Ser Glu Arg Ser Pro Ala Tyr Thr | |
| 1010 1015 1020 | |
| Pro Tyr Ala Gln Val Val Leu Asn Tyr His Ser Glu Phe Glu Gly | |
| 1025 1030 1035 | |
| Gln Glu Gln Ala Ala Leu Ala Pro Asp Gly Asp Ala Leu His Ile | |
| 1040 1045 1050 | |
| Glu Gly Arg His Ala Ala Ser Val Gln Tyr Ala Ala Phe Asp Leu | |
| 1055 1060 1065 | |
| Lys Ile Glu Met Asn Arg Val Gly Ala Glu Leu Asp Leu Val Phe | |
| 1070 1075 1080 | |
| Glu Tyr Ser Thr Ala Leu Phe Asp Gln Ala Thr Ile Ala Arg Leu | |
| 1085 1090 1095 | |
| Ala Gly His Tyr Val Arg Val Leu Glu Gln Val Gly Ala Asp Ala | |
| 1100 1105 1110 | |
| Gln Ala Arg Val Ala Ala Leu Ala Leu Leu Ser Glu Gly Glu Leu | |
| 1115 1120 1125 | |
| Ala Ala Leu Ser Ala Gln Trp Gln Ser Ala Arg His Asp Tyr Pro | |
| 1130 1135 1140 | |
| Arg Thr Ala Asn Leu Ala Thr Leu Leu Glu Gln Gln Ala Ala Arg | |
| 1145 1150 1155 | |
| Thr Pro Asp Ala Pro Ala Val Ala Cys Ala Gly Thr Val Leu Thr | |
| 1160 1165 1170 | |
| Tyr Ala Gln Leu His Gly Arg Ala Asn Arg Leu Ala His Leu Leu | |
| 1175 1180 1185 | |
| Arg Ala Arg Gly Val Gly Pro Asp Val Leu Val Gly Val Cys Val | |
| 1190 1195 1200 | |
| Glu Arg Ser Leu Asp Met Val Val Ala Val Leu Ala Val Val Lys | |
| 1205 1210 1215 | |
| Ala Gly Gly Ala Tyr Leu Pro Leu Asp Pro Asn Tyr Pro Ala Ala | |
| 1220 1225 1230 | |
| Arg Leu Ala Tyr Met Leu Glu Asp Ala Ala Pro Ala Leu Val Leu | |
| 1235 1240 1245 | |
| Thr Gln Gln His Leu Ala Ala Arg Leu Pro Ala Gln Ala Pro Ala | |
| 1250 1255 1260 | |
| Ile Val Ile Asp Ala Asp His Thr Ala His Pro Asp Ser Ala Pro | |
| 1265 1270 1275 | |
| Ala Pro Val Gly Gly Pro Asp Asp Leu Ala Tyr Val Ile Tyr Thr | |
| 1280 1285 1290 | |
| Ser Gly Ser Thr Gly Lys Pro Lys Gly Ala Met Val Gln Arg Gln | |
| 1295 1300 1305 | |
| Gly Val Leu Asn Leu Leu Thr Trp Phe Val Arg Glu Tyr Ala Ile | |
| 1310 1315 1320 | |
| Gly Ala Ala Asp Arg Val Leu Leu Val Ser Ser Phe Ser Phe Asp | |
| 1325 1330 1335 | |
| Leu Thr Gln Lys Asn Ile Phe Gly Ile Leu Leu Val Gly Gly Glu | |
| 1340 1345 1350 | |
| Leu His Leu Met Ala Asp Asp Tyr Ala Pro Glu Arg Ile Gly Ala | |
| 1355 1360 1365 | |
| Tyr Ala Gly Thr Ala Gly Ile Thr Leu Ile Asn Cys Ala Pro Ser | |
| 1370 1375 1380 | |
| Ala Phe Tyr Pro Leu Leu Ala Asp Gly Gly Ala Ala Arg Met Ala | |
| 1385 1390 1395 | |
| Ser Leu Arg Ala Val Phe Leu Gly Gly Glu Pro Ile Gln Val Gly | |
| 1400 1405 1410 | |
| Leu Leu Arg Ala Ala Tyr Arg Asp Val Ala Thr Pro Pro Leu Val | |
| 1415 1420 1425 | |
| His Asn Thr Tyr Gly Pro Thr Glu Ala Ser Asp Val Val Ser His | |
| 1430 1435 1440 | |
| Tyr Ala Trp His Pro His Glu Pro Val Thr Thr Leu Pro Ile Gly | |
| 1445 1450 1455 | |
| Arg Ala Ile Ala Asn Thr Arg Leu Tyr Val Leu Asp Gly Gly Arg | |
| 1460 1465 1470 | |
| Gln Leu Val Pro Gln Gly Ala Val Gly Glu Leu Tyr Val Gly Gly | |
| 1475 1480 1485 | |
| Asp Gly Val Gly Arg Gly Tyr Leu His Arg Pro Glu Leu Thr Ala | |
| 1490 1495 1500 | |
| Glu Arg Phe Leu Pro Asp Pro Phe Ala Gly Gln Pro Gly Ala Arg | |
| 1505 1510 1515 | |
| Met Tyr Arg Thr Gly Asp Leu Val Arg Ser Leu Pro Asp Gly Val | |
| 1520 1525 1530 | |
| Leu Glu Tyr Leu Gly Arg Ile Asp His Gln Val Lys Val Arg Gly | |
| 1535 1540 1545 | |
| Leu Arg Ile Glu Leu Gly Glu Ile Glu Glu Ala Leu Ala Ala Leu | |
| 1550 1555 1560 | |
| Pro Ala Ile Asp Gln Ala Leu Val Leu Ala Cys Asp Asp Leu Ala | |
| 1565 1570 1575 | |
| Ala Asp Val Arg Leu Val Ala Tyr Leu Val Gly Val Asp Ala Gln | |
| 1580 1585 1590 | |
| Ala Ala Leu Asp Pro Val Ala Leu Arg Ala Ala Leu Thr Gln Thr | |
| 1595 1600 1605 | |
| Leu Pro Gln Tyr Met Leu Pro Ser His Phe Val Gln Leu Pro Ala | |
| 1610 1615 1620 | |
| Phe Pro Leu Ser Pro Asn Gly Lys Val Asp Arg Ala Ala Leu Pro | |
| 1625 1630 1635 | |
| Arg Pro Val Gln Asp Leu His Ala Pro Phe Val Ala Pro Ser Gly | |
| 1640 1645 1650 | |
| Ala Thr Glu Gln Ala Leu Ala Gln Ile Trp Ala Glu Val Leu Lys | |
| 1655 1660 1665 | |
| Cys Ala Asp Val Gly Arg Ala Asp Asp Phe Phe Gln Leu Gly Gly | |
| 1670 1675 1680 | |
| His Ser Leu Leu Ala Thr Gln Val Met Ser His Val Arg Ala Arg | |
| 1685 1690 1695 | |
| Leu Gly Val Asp Leu Pro Leu Arg Thr Leu Phe Glu Tyr Pro Thr | |
| 1700 1705 1710 | |
| Leu Ala Ala Leu Gly Glu Gln Ile Asp Arg Ala Asp Lys Ala Ala | |
| 1715 1720 1725 | |
| Ser Gly Pro Leu Ala Leu Ala Ala Gly Asp Gly Ala Ala Ala Gly | |
| 1730 1735 1740 | |
| Ala Leu Ala Pro Leu Ser Tyr Ala Gln Gln Arg Leu Trp Val Leu | |
| 1745 1750 1755 | |
| Gln Lys Leu Gly Glu Asn Pro Ala Val Tyr Asn Leu Pro Phe Ala | |
| 1760 1765 1770 | |
| Val Glu Leu Glu Gly Ala Val Asp Val Pro Ala Leu Gln His Ala | |
| 1775 1780 1785 | |
| Leu Asp Leu Leu Ala Arg Arg His Ala Ala Leu Arg Thr Ala Phe | |
| 1790 1795 1800 | |
| Val Thr Val Asp Gly Glu Pro Leu Cys Ala Val Ala Ala His Ala | |
| 1805 1810 1815 | |
| Ala Leu Pro Leu Gln Thr Ala Ser Leu Ala Asp Ala Ala Pro Gln | |
| 1820 1825 1830 | |
| Ala Val His Asp Trp Leu Val Ala Ala Ala Gln Val Pro Phe Asp | |
| 1835 1840 1845 | |
| Leu Glu Cys Ala Pro Leu Ala Arg Ala Thr Leu Leu His Val Ala | |
| 1850 1855 1860 | |
| Pro Ala Arg His Val Leu Leu Leu Val Met His His Ile Ile Ala | |
| 1865 1870 1875 | |
| Asp Gly Trp Ser Ile Gly Val Leu Ser Arg Glu Leu Ser Val Leu | |
| 1880 1885 1890 | |
| Tyr Asn Ala Ala Arg Arg Gly Val Pro Ala Ala Leu Pro Ala Leu | |
| 1895 1900 1905 | |
| Pro Leu Gln Tyr Ser Asp Tyr Ala Arg Trp Gln Arg Ser Arg Ala | |
| 1910 1915 1920 | |
| Glu Glu Gly Ala Phe Asp Asn Gln Leu Ala Tyr Trp Arg Asp Arg | |
| 1925 1930 1935 | |
| Leu Ala His Ala Pro Ala Met Leu Ala Leu Pro Leu Asp His Pro | |
| 1940 1945 1950 | |
| Arg Pro Ala Leu Pro Ala Leu Arg Gly Asp Val Leu Ala Phe Thr | |
| 1955 1960 1965 | |
| Val Glu Pro Gly Leu Leu Ala Gly Leu Arg Arg Leu Ala Arg Glu | |
| 1970 1975 1980 | |
| Gly Gln Ala Ser Leu Phe Met Val Leu Ser Ala Ala Phe Gly Val | |
| 1985 1990 1995 | |
| Leu Leu Gly Arg Tyr Ser Gly Gln Arg Asp Leu Cys Ile Gly Thr | |
| 2000 2005 2010 | |
| Pro Ile Ala Asn Arg His His Gly Glu Leu Glu Gly Leu Val Gly | |
| 2015 2020 2025 | |
| Phe Phe Val Asn Thr Leu Val Leu Arg Leu Thr Leu Glu Pro Ala | |
| 2030 2035 2040 | |
| His Gly Phe Glu Ala Leu Leu Ala Gln Val Arg Glu Thr Val Leu | |
| 2045 2050 2055 | |
| Gln Ala Phe Ala Asn Gln Asp Leu Pro Phe Glu Gln Val Val Ala | |
| 2060 2065 2070 | |
| Ala Ser Ala Gly Ala Arg Gln Ala Gly Gln Thr Pro Leu Phe Gln | |
| 2075 2080 2085 | |
| Ala Met Leu Ala Leu Gln Asn Ala Pro Gln Asp Glu Val Ala Leu | |
| 2090 2095 2100 | |
| Glu Ala Leu Ser Gly Arg Val Leu Asp Val His Asn Gly Gly Ala | |
| 2105 2110 2115 | |
| Lys Phe Asp Leu Thr Leu Asp Ile Thr Pro Arg Gly Asp Arg Leu | |
| 2120 2125 2130 | |
| Asp Cys Arg Phe Glu Tyr Asp Cys Ala Leu Phe Glu Arg Ala Thr | |
| 2135 2140 2145 | |
| Val Ala Arg Leu Ala Asp Asn Leu Leu Thr Leu Leu Ala Ser Ile | |
| 2150 2155 2160 | |
| Val Ala Ala Pro Gln Ala Pro Leu Gln Thr Leu Ala Leu Leu Ala | |
| 2165 2170 2175 | |
| Pro Ala Glu Gln Ala Leu Leu Ala Arg Leu Gly Ala Gly Thr Pro | |
| 2180 2185 2190 | |
| Ala Gly Ala Ala Pro Leu Val His Arg Ala Phe Glu Ser His Ala | |
| 2195 2200 2205 | |
| Ala Arg Asn Pro Asp Ala Val Ala Leu Thr His Glu Gly Thr Thr | |
| 2210 2215 2220 | |
| Leu Thr Tyr Ala Glu Leu Asn Ala Arg Ala Asp Thr Leu Ala Arg | |
| 2225 2230 2235 | |
| Ala Leu Thr Ala Ala Gly Val Gly Pro Asp Ser Arg Val Val Leu | |
| 2240 2245 2250 | |
| Tyr Ala Glu Arg Gly Ile Gly Leu Ile Thr Gly Val Leu Ala Ile | |
| 2255 2260 2265 | |
| Leu Lys Ala Gly Gly Ala Tyr Val Pro Phe Asp Pro Ala Tyr Pro | |
| 2270 2275 2280 | |
| Arg Glu Arg Leu Ala Tyr Met Ala Gln Asp Cys Met Pro Ala Ala | |
| 2285 2290 2295 | |
| Leu Val Thr Glu Pro Ala Leu Leu Ala Glu Ala Gln Ala Leu Gly | |
| 2300 2305 2310 | |
| Pro Ala Leu Ala Ala Val Pro Cys Cys Leu Ile Glu Ala Gly Gly | |
| 2315 2320 2325 | |
| Ala Gln Pro Gly Ala Ala Pro Ala Pro Ala Ser Gly Ala Ala Val | |
| 2330 2335 2340 | |
| Gly Pro Gly His Leu Ala Tyr Met Ile Tyr Thr Ser Gly Ser Thr | |
| 2345 2350 2355 | |
| Gly Gln Pro Lys Gly Val Gln Val Glu His Gly Gly Leu Ala Ser | |
| 2360 2365 2370 | |
| Leu Ala Ala Asp Gln Asn Arg Ala Leu Ala Ile Gly Pro Gly Ser | |
| 2375 2380 2385 | |
| Arg Val Leu Gln Phe Ala Ser Ile Ser Phe Asp Ala Ser Ile Trp | |
| 2390 2395 2400 | |
| Glu Ile Val Met Ala Leu Ala Ser Gly Ala Ala Leu Val Ser Ala | |
| 2405 2410 2415 | |
| Pro Arg Ala Ala Leu Met Pro Gly Ala Pro Leu Leu Ala Phe Leu | |
| 2420 2425 2430 | |
| Gly Glu Gln Asn Ile Ser His Ala Leu Leu Pro Pro Ser Val Leu | |
| 2435 2440 2445 | |
| Ala Ile Met Ala Asp Asp Glu Arg Leu Ala Pro Met Thr Leu Leu | |
| 2450 2455 2460 | |
| Val Gly Gly Glu Ala Cys Pro Pro Ser Val Ala Ala His Trp Gly | |
| 2465 2470 2475 | |
| Arg Arg His Arg Phe Val Asn Ala Tyr Gly Pro Ser Glu Ile Thr | |
| 2480 2485 2490 | |
| Val Cys Ala Thr Thr Trp His Tyr Asp Gly Arg Ala Gly Gly Ala | |
| 2495 2500 2505 | |
| Ile Pro Ile Gly Arg Pro Leu Ala Gly Thr Arg Ile His Ile Leu | |
| 2510 2515 2520 | |
| Asp Glu Ala Gly Gln Pro Val Pro Val Gly Ala Val Gly Glu Ile | |
| 2525 2530 2535 | |
| His Ile Gly Gly Val Gly Val Ala Arg Gly Tyr Leu Asn Arg Pro | |
| 2540 2545 2550 | |
| Asp Leu Thr Ala Gln Arg Phe Leu Ala Glu Pro Gly His Pro Asp | |
| 2555 2560 2565 | |
| Thr Arg Leu Tyr Arg Thr Gly Asp Leu Gly Arg Trp Asp Ala Ala | |
| 2570 2575 2580 | |
| Gly Met Leu His Tyr Ala Gly Arg Asn Asp Phe Gln Val Lys Val | |
| 2585 2590 2595 | |
| Arg Gly Phe Arg Ile Glu Leu Gly Glu Ile Glu Ala Val Leu Arg | |
| 2600 2605 2610 | |
| Ala Gln Pro Ala Leu Ala Asp Ala Ala Val Ile Ala Arg Ala Gly | |
| 2615 2620 2625 | |
| Ala Asp Gly Gln Gln Arg Leu Leu Ala Tyr Val Val Pro Arg Ala | |
| 2630 2635 2640 | |
| Asp Thr Ala Pro Glu Pro Ala Ala Leu Arg Ser Ala Leu Leu Ala | |
| 2645 2650 2655 | |
| Arg Leu Pro Asp Tyr Met Val Pro Gly Ala Phe Ile Ala Leu Pro | |
| 2660 2665 2670 | |
| Ala Leu Pro Gln Thr Pro Asn Gly Lys Leu Asp Arg Asp Ala Leu | |
| 2675 2680 2685 | |
| Pro Leu Pro Asp Asp Asp Ala Leu Ala Arg Gln Ala Phe Val Pro | |
| 2690 2695 2700 | |
| Pro Gln Asp Gly Ile Glu Arg Arg Leu Ala Asp Ile Trp Gln Gly | |
| 2705 2710 2715 | |
| Val Leu Gly Val Ala Ala Val Gly Arg Phe Asp His Phe Phe Glu | |
| 2720 2725 2730 | |
| Leu Gly Gly His Ser Leu Ala Leu Thr Lys Leu Ser Phe Leu Val | |
| 2735 2740 2745 | |
| Gln Glu Ala Phe Gly Val Thr Leu Ser Leu Gly Gln Leu Tyr Gln | |
| 2750 2755 2760 | |
| Leu Gln Gln Leu Ala Gln Gln Ala Asp His Ile Ala Ala Ala Leu | |
| 2765 2770 2775 | |
| Ala Thr Ala Ser Arg Lys Lys Val Leu Val Leu Asp Leu Asp Asp | |
| 2780 2785 2790 | |
| Glu Glu Glu Ala Ala | |
| 2795 | |
| (Nucleotide Sequence of eppB) | |
| SEQ ID NO 5 | |
| atgaaactcc atgaactgat ctcccatctg catgccaccg gcgtctcggt gcagaaccgc | 60 |
| gacggcaagc tgcaggtgac gagcgccgac ggcgacctgc ccgacgccac gctggcggcg | 120 |
| ctgaagaagc acaagaagga cgtggccgca tactatgccg agcccgcgcc ggtcgatgtc | 180 |
| gcggcaccgg aacgggagca gccactttcg ttcgcgcagc gccgcctgta tttcctgtac | 240 |
| cagtacgagc cggccgcgac gcacttcaac ctgccgatgg agctcggcat cgagggcgcc | 300 |
| ctcgacagcg agcgcctgcg cggcgcgctg ctcgacgtgg tgcagcgcca tcccatctac | 360 |
| cgcaccacgt atggcatgcg cgacggcgtg ccattccagc gcgtgcgcag cgacctgcag | 420 |
| cccaccctcg ggctggacga cctgcgccac ctcgatgccg ccgctgccga tgaacggatg | 480 |
| gcgctgcagc gcgcacgtat tgccgccacg ccattcgacc tggccaacga gctgccgctg | 540 |
| cggatgcacc tgttccgcca gggcgaggcg gcgtattcgc tgctgatcgt gttccaccat | 600 |
| atcgcgaccg acgaatggtc gatccagcag ctgatgcgcg aactgtcgga cgcctatcgg | 660 |
| ggcaccggcc ccgccgcgcc ggtgccggcg tacggtgaat acgtcgcctg gcagaacagc | 720 |
| cggcatgcgg ggcgcggcta cgaagcggcc cggtcctact ggaccgaaca cctggccgac | 780 |
| gcggagcccg tgctggcatt gccggcggac cgcgcgcgcc cgtcacgcca gacctaccgc | 840 |
| accggcctcg agcggcttgc gttgccggcg gccttgcgcg aacgcgccag ccagtgcgcc | 900 |
| ggccggctcg gcatctccga gttcgcgctg tatctcggcc tgtaccaact gctgctgcac | 960 |
| cgcctgacgg ggcagcgcga cctcgtggtc ggcacggacg tgttcggccg cgatcacggc | 1020 |
| cggttccgcg aggtggcggg cttcttcgtc aatcagctgg cactgcgcca gcaggtcccg | 1080 |
| gccggcgccc aggccgatga attcctgcgc caggtggcgc gcgacgtcaa cgatgcgatg | 1140 |
| ctgttccagg acctgccgtt cgaccagctg gtcgacgctt tgcaggtgga gcgcgacccg | 1200 |
| gcctattcgc cgctgttcca ggtgaagttc ctgtaccgcc gcaacagcct gacgccggac | 1260 |
| ctgttcgacg gcctgcgcag ctggaacaag gagatgttcg cggtacagtc ccagtacgac | 1320 |
| ctgacgctgc aggtgctgcc ggacacggtg gaagcgtatt tcaacccgga cctgttcgac | 1380 |
| gcggcgcgcg tggccggctg gctggaactg tatgtggcgc tggccgagga ggtcgtggcc | 1440 |
| gacccggcgc agccgcttgc cggcctgctc gatgcgcgcc tgcgcgccat ggtcgcaccg | 1500 |
| ttcagccatg gcgaggcgac cggcccggcc gggctggcgc tgtgcgaccg catcgccagc | 1560 |
| tgggcgggtg ccacgccgga gcgtgtcgcc atcggcagcg ccgaaggcga cctgacgtac | 1620 |
| gccgaactgg tacgccgcat ggaggccgtg gccgggcaac tggcggcgct gggcaccggc | 1680 |
| cgcggcgaca aggtggcggt ctatctcgac cgttcggccg acctggtggt cgccgtgctg | 1740 |
| gcgatcgccc gcgtgggcgc ggtgctggtg ccgctcgaca cggacaatcc accggagcac | 1800 |
| atcgcgttcg tgctgcacga cagcggtgcc aacgtggtgc tgagcgaaag cctgcgggcc | 1860 |
| gacgacatcg tcgatttcta tgggctgtgg ctggacatcg gcgcgctgag cgcggcgccg | 1920 |
| gcaccgcagg cgctgcccgc atacgacacg ctgcaaggcg acgacctggt ctaccagctg | 1980 |
| tacacctccg gctcgacggg gcggccgaag ggcgtgctcg tcacgcgcgc cggcttcgcc | 2040 |
| aatctgtgcg actggtatgc ctcgttcgcc cgaatcggcc ccgacagccc ggtgctgttg | 2100 |
| atgattccga tcggcttcga cgcttcgctg aagaacatct tcacgccctt gatgcagggc | 2160 |
| gcgacgctgg tgctggcacc ggcggcgccg ttcgatccgg atgccctgct ggcgctgatc | 2220 |
| gccagccgcg gcgtggccgt ggtgaacacg gcgccgagcg cgctgtatgc gctgctgcag | 2280 |
| caggacgcgc cgcgccagta cgcggcgctg gccgggctga ccatgttcgc cgtcggcggc | 2340 |
| gaggcgctgg acctggggct ggtacgcccg tggctggaca gcccgaactg ccgtgcgctg | 2400 |
| ctggccaata tctatggccc gaccgagtgc accgatatct cgctggcgtt cgcggccgat | 2460 |
| gccgcgacct ggctggcgcg cgccacggtg acgatcggcc ggccgatccg caacacccag | 2520 |
| gctttcatcg tgaacgacga gctggcgctg tgcccacccg gcacgccggg cgaactggtg | 2580 |
| attgccggct gcggcgtcgc gcgcggctat caccagctgc cggacgcgga tgcgcgcagc | 2640 |
| ttcgtgcacg ccgcgctggc acaggggcgt atctatcgca ccggcgacta tgcctgccat | 2700 |
| gaggccgacg gcaatgtgct gtacctgggc cgccgcgacg gccagatcaa gatccgcggc | 2760 |
| aagcgggtgg agacgggcga agtgctggcg caaatggcgc gcctgctgcc gggccgcacg | 2820 |
| ctgagcgtgc agcgctatgc gcgcgaccgc gtcgagatgc tggtgggctt cgtggcgggc | 2880 |
| cgtccg atctggacag cgtgcagctg cgtgccgaac tggcgcgcca cctgccgcgc | 2940 |
| cacgcggtgc cggccgatat cgtcttcgtg ccgtcgatgc cgctgagtgc caacggcaag | 3000 |
| atcgcggcgg cggcgttgct ggcgctgtac gaggaacacc gcagcacccg ccagtccgcc | 3060 |
| acgcgc cgcgc tgagtgcgac cgaagcggcc atcgccgcga tctggcacca gttgctgggc | 3120 |
| gaggtcgcgg tggaggcgga cagcagcttc ttcgccgtcg gcggcgactc gatcttctcg | 3180 |
| atccagctgg tggcggaatt gcagaagctg gggtacgcgg tcgcggtggc cgacatcttc | 3240 |
| aaatacccgg tactggaaca gctggccgcg ttctgcgaca gtcgttcgca tgtcgccgtc | 3300 |
| acgaccacgg aggcgctcgc accgttcgcg ctggtcgacc cggccgacct ggccgctctg | 3360 |
| ccggaagggc tggaggatgc ctacccggtc acgtcgctgc agcaggggat gctgttccac | 3420 |
| tgccggatgg agccggacag tgcgatgtac cacgatgtct tcagctacga gctgcgtttc | 3480 |
| gactacgatg ccgccctgct gaagcaggcc gtggggctgg tgctggccca caaccaggtg | 3540 |
| ctgcgtaccg gcttcgaact cgataccgtg tccgagccgc tgcaactggt gtatgcgcgc | 3600 |
| gtcgagccgg agtggtcgga gcaggacctg cgccacctgt ccgcggcgga gcaggaggcg | 3660 |
| gcggtggcca cggccatcgc ttcgctcaag cgcaccggtt tcgccctgtc cggcccgagc | 3720 |
| ctgatccgct tcaccgtgtt gcgcaaggcc gagggctgca tccagctgct gatcgacgcg | 3780 |
| caccacgcga tcctggacgg ctggagcatg gcaacgctgc agcggcagat cttcgagcac | 3840 |
| tacggccatc tgcgcttcgg cctgccgctg gccgacgtct tcgacacggg cgggttgcgg | 3900 |
| ttcgccgact acgtcgccca gcaggccgcg gccgagcagg acgacgccgc ggccgcgcac | 3960 |
| tggcgtacgt attgccgcgc cgccggcagc ggcgcgctgt cggcgcggct gccgcaacag | 4020 |
| ggcgaagcgg tgttgcacac gctgcccttg ccggcggacc tgcccgcacg cctggcgcaa | 4080 |
| cgtgtcgcga ccgatggcgt gatgctgaaa acgctgctga tgatggcgca cgcgtacatg | 4140 |
| ctgcgcgcgc tcctgccgag tgagcgcctc agcacggcgc tgacggacaa cggccggccc | 4200 |
| gaaacgccgg gcgcgcagaa catcgtcggc ctgttcgtca acgtgctgcc ggtggccttc | 4260 |
| gacctggacg ccagctggcg ccagctggcc gccgcgttgc aggcggacga ggtggcgcgc | 4320 |
| aagccgttcc ggcgcttccc gttcgcgcac atcgtgcgcg aacaacgggc gctgcagatc | 4380 |
| gacacgctgt ttacctacaa taacttccat gtcagcgagg cgctgcaggc ggccgagtgg | 4440 |
| ctgcagatcg agccgggcaa cagctatgag gaaaccaatt tcaagctggc ggtgctggtc | 4500 |
| aacggcaacc tgcagagcgg cctgacgctg acgctcgaaa gccgcctggc gctgacggcg | 4560 |
| gcgcaggtcg caacgctgca gcgcgagttc gtgttcgccc tcgactgcat ggcacaggcg | 4620 |
| ttcgacgcgc cgatcccgca gcgtgccgat cgcctgctgc ccgtgctggc gcaggccggt | 4680 |
| gcggcagtgg cccggttgcg ctggcagggc gtcgccccgg cggcggtgct ggaggcggcg | 4740 |
| ctggcccgtt gcgccctgcg tgtcgcggca atcgagcgcg cgccggcaca ggcgccgttc | 4800 |
| gatatcgccg ccagcgtgga gcaggacggc cagcggctgg agtggcggat cgcgccggag | 4860 |
| tgggcgcagc atcccgacct gccggccctg ctgtccgaaa cgatggaacg cgtgctggcg | 4920 |
| acaggtgcgc ccgcgggcga cgtcgccgtc gcttgcgatg cgcagggagc ggcatggccg | 4980 |
| ctgcgccagc tggaagacga catggcgttc tggcggggcc ggctggccga agcgccagcg | 5040 |
| cacctgaacc tgccgcaaac gctggcgctg gccgcgggcg cggaacgcac ggacgagcgg | 5100 |
| catgtgcggg ccgtcgatac ggcggcgctg gcggccctga ccgcgcgcac cgggctgtcc | 5160 |
| cgcggcgcca tcctgctggg ggcatggctg gcactgctgg cgcgcctgag cgggcaggaa | 5220 |
| accgtgctga ccggcgtacg cctgcgcgcc ggcggaccgt tgctgccgct ggtggccgag | 5280 |
| accggcgacg acccggctgc aacggtcctg ctgacgcgtg ccgctggcgc gctgcaggcc | 5340 |
| tgcgccgcac acgccggcgt gcccgccagc ctgctgccgg cacgccatgc ggccgcgttc | 5400 |
| gcgctggccg atgacggccc gctgccggcc gacatggcga tcgtcgcgac cgacgacggc | 5460 |
| gcctgccgcc tcgaactggc ggccgatgtc catgacgccg ccggcgccga ccggctcgcg | 5520 |
| gccaacctgg ccgagctgtt gcaaggcgcc gccgccgcgc cgggcgagcg gctgtcgcgc | 5580 |
| ctgccgctgc tgggcgcggc ggagcgccac cgcgtgctgg tgcaattcaa cgacagcgcc | 5640 |
| cagcacttcg acgacacccg ccagttgcac cagatggtcg aagaccaggc cgccgccgat | 5700 |
| cccggcgcgc tggccctgct gtacggcagc gacacgatga cgtacgaggt gctgaaccgc | 5760 |
| cgtgccaacc aggtggcgca attcctgcac ggccatggca tcggtgccaa cgaccgcgtc | 5820 |
| gccgtctgca tggagcgtgg cctggagatg gtggtcgcga tcctcggcgt gctcaaggcc | 5880 |
| ggcgccgcct acatgccgct cgacccggcc tatccggtcg agcgtatcgc ctatatgctc | 5940 |
| gacgacagcg cgccccgggc gctgctggcc caggcgccgc tgctggcggc cttggagccg | 6000 |
| gtgcgccggc tggcggccga gctgccttgc ctgctgctgg ccgaaggcct ggcggtgctg | 6060 |
| gacgggctgc cggatgcgaa cccgcccgcg ccgccgctgg cgcaggccgc agccaacctg | 6120 |
| atgtacgtgc tgtacacgtc cggctcgacc ggccggccca aaggggtcgc gatggcccag | 6180 |
| ggcccgctgg tcaacctgat ccggtggcag gcttcgtcgc gttcgaagct ggcccagcgc | 6240 |
| gaacgcacgc tgcagttctc cgccctgggc ttcgatgcca cgttccagga gatcttcagc | 6300 |
| gcattgtgct atggcgccag cctggtgctg ctggccgagt ccatccggcg cgatccgcgc | 6360 |
| gaactggtgc ggctgatgcg ccggtacgac gtggaacgca ttttcctgcc gttcgtcgcg | 6420 |
| ctgcagaaca tcgccgaggc ggcggtggag ctgggcgaac cgttgcctgc gctgaacacg | 6480 |
| atgatcacgg caggcgaaca gttgcgcatc agtcccgcca tcgtgcagtt cttccgcatg | 6540 |
| cgcgccggcc gcagcctgca caactactac ggcccgaccg agagccacgt cgtgacgacg | 6600 |
| tatgtgctgg acggcgatcc gggcgcgtgg cccgcgttgc cgccgatcgg cgcgccgatc | 6660 |
| gccaacaccc agatctacat tctcgacgcg gcgctgcagc cggtggccct gggcgcgcat | 6720 |
| ggcgagctgt atatcgccgg cgattgcctg gccgacggct acctgaaccg gcctgacctg | 6780 |
| acggcggagc gcttcgtcgg caatgtcttc cggccaggca cgcgcatgta caagacgggc | 6840 |
| gacatcgccc gctggctgga ggacggcaat atcgaatacc tgggccgcaa cgacagccag | 6900 |
| gtcaagatcc gcggctaccg catcgagccg ggcgaggtcg aggcggcact ggccgcgtgc | 6960 |
| gccggcgtgc gcgaggcggt cgtggtggcg cgcgaagacg tgccgggaca gaagcgcctg | 7020 |
| gtggcgtatc tgctggccca gccaggccac acgctggcac cggcggcgct gcgcgaccgg | 7080 |
| ctggccaccg tgctgccgga ctacatggtg ccggccgcct ttgtctgcat gacggcgttc | 7140 |
| cccgtcagcc cgaacggcaa gctggaccgg cgcgcgctgc cggcgcccga cgccgccgcg | 7200 |
| caattgcgcc agccgtacga agcgccgcaa ggaagcaccg aaacggcgct ggcggcgatc | 7260 |
| tgggaagacc tgctggccgt acgcgacgtt ggccgccgcg accacttctt cgaactcggc | 7320 |
| ggccactcgt tgctggccgt gcggctgacc acgcgcgtac gccaggtact gcagcgtgag | 7380 |
| ctggcgctgc gggcgttgtt cgagcagccg gtgctggccg atctcgcccg cgtcgtcgat | 7440 |
| ggcctggaca gcgccggtac cgcaccgctg cgcgcgttgc cgcgtacgcc cgaccaggtg | 7500 |
| ctgcccctgt cgttcgcgca gcagcgactg tggttcgtgc aggagctcga aggtcccacg | 7560 |
| ccgacctaca acatgccggc cgcgctgcgc ctgacggggc ggctggatgc cgccgcgctg | 7620 |
| gagccggcgc tgcaatacct gatcgagcgc cacgaggtcc tgcgcaccaa cttcgacagc | 7680 |
| gtggagggcg tgccgcacct gcgcatcgcg ccgtcgcgta ccgtgacgct ggccgttacc | 7740 |
| gacgtcgcgc cggacgaggt ggaggcgcgt gccgcgcgcc atgcggcgct gccgttcgac | 7800 |
| ctggcgcgcg agcccttgct gcgtgccgaa ctgctgcggc tgtcggccga ccagcacgtg | 7860 |
| ctgctgctga acgtgcacca tatcgtcagc gacggctggt cgctgaacat cctggccgac | 7920 |
| gaatggctgc gtgcgtacga cgccctgcgc gccggccgcg cgccggcgct gccggtgctg | 7980 |
| ccgctgcagt acgccgacta tgcgtactgg cagcgcgaac aactgaccga agccgtgcgc | 8040 |
| gagcgccagc tggcctattg gaccgggcaa ctggccggtg cgccggagct gctcgacctg | 8100 |
| ccgaccgacc gcgtgcggcc ggcggtgcag cgcttcgatg gcggcgatga acagctgcgg | 8160 |
| ctggacccgg cgctgtcgca cgccgtgcgc cagctggggc atgcgcacaa tgccagcctg | 8220 |
| ttcatgacgc tggtcacggc gttcggcctg ctgctgggcc gtctcagcgg ccaggacgac | 8280 |
| gtgctggtcg gcgtgccgca ggccacccgc gaccggcgcg agctggaggg catgctcggc | 8340 |
| atgctgctgg gtaacctggt cctgcgcatg cgcctggacg acgcggccgg tttcggcacg | 8400 |
| ctgctggagc aggtgcgccg caccgcgctg gaggcttacg aacacagcgc catcccgttc | 8460 |
| gagcaggtcg tcgacgcgtt gccgctgcag cgtgacctga gccgcaatcc gctgttccag | 8520 |
| gtcttcttca acatgctcaa cctgccggag acgaactata cgtcgccgga gctggcgatc | 8580 |
| gaaggactgc aaagcacgct gctggacgcc aagttcgacc tgacgctgta tgcgcaggac | 8640 |
| agcgaagaag gcatcctgct gcacctggtg tacaaccgtg gcctgttcga tgcgcagcgc | 8700 |
| atgcgcgaat tgctgcggca gtaccacagc ttgctggagc aggtcagcca ggcgcccgcc | 8760 |
| atcgcctgca aggccgtgtc gctgctgacg gcgccagcgc gcgcggtcct gcccgatccg | 8820 |
| gcggtcgtcc tggatgcgac ctggcacggc agcattcccg gccgctttgc cgcgctggtg | 8880 |
| gcggcgcagc cggcggcgct ggccgtcacg gcggcgcacc tgcagtggac ctacgcggaa | 8940 |
| ctggacgagc gcagcgaggc cgtggcctgc tggctgcagg aggccggcgt cggcgccggc | 9000 |
| gccgtggtgg cgatctgcgc cgcccgccgc gcggcgctgg tgccggccgt gctgggcgtg | 9060 |
| ctgaaggcgg gtgccgccta taccatcgtc gatcccgctt acccggccga gcacgtgcgc | 9120 |
| gcctgcctgg ccgtggcccg gcccgccgcg tggctgacgg tggccgaggg cggcgatgcc | 9180 |
| gcattgcttg cctgcctgcc cgcgccggtg ccgcgactcg atctgagcgg gaacgatggc | 9240 |
| tggccggtgc tggcagcggg cgtgcgtgcc gtgccggccg cctggacggc cgacgacgtc | 9300 |
| gccgtgctga cgttcacgtc cggctccacc ggcctgccca aggccgtcga aggccgccac | 9360 |
| ggcgcgctga cgcacttcta cccatggctg caacaacact tcggcatggg gccgcaggat | 9420 |
| cgctacgcac tgttgtcggg cctcgcgcac gacccgctgc agcgcgatat cttcaatacc | 9480 |
| ttatggatgg gcgccagcct gcacgtgccg ccggtggacg ccatcggccc gggcctgctg | 9540 |
| gccgactgga tggcggccga gaacatcagt gccgtcaacc tgacgccggc catgctgcag | 9600 |
| ctgctgtgcc aggacgcacg cgctctgccg acattgcggc atgccttcct ggtgggcgat | 9660 |
| atcctgacgc aggccgacgt ggccctgctg cagcaggtgg cgccgcgctg cgccgtggtc | 9720 |
| agctactacg gcgccaccga ggcgcagcgg gcgttcggca tggtggagat cgccccgggt | 9780 |
| acggcggctg gcctgacgcg cgacgtcatc gcgctgggcc acggcatccc cggcgtgcag | 9840 |
| ctgctggtgc tgaacggcgc cggcacgctg gccgggatcg gcgaggtggg cgaagtgtgc | 9900 |
| atccgcagcc cgcacctggc gcgcggctac cgcgacgacg cggcgatgac ggcacgccag | 9960 |
| ttcgtcgcca acccgttcgg tggcggcgac cgcctgtacc gcacgggcga cctgggacgc | 10020 |
| tatctgcccg acggcatggt ggcgggcctg ggccgcaacg accagcaggt caagctgcgc | 10080 |
| ggcttccgca tcgagctggg ccacgtcgag gccgcgctgg cccggctgcc gcaagtgcgc | 10140 |
| gaagccgtgg tgctggcgtt gggcagcggc gaggcgcgtc gactggtcgc atacgtcgtc | 10200 |
| ccgcgcggca ccttcgatgc cgacgcggcc gcggcggcct tgcgcggcac cttgcccgac | 10260 |
| tatatgcggc cggccgccta cgtggtcctc gagcgtctgc cgctgacgcc caacggcaag | 10320 |
| ctcgatcgtc gtgcgctgcc cgcgccggcg gccacgcccg cggtggcgga cacggcgccg | 10380 |
| gcgacggcac tggaagcctc gctgtgcgcg ctcatggccg agctgctgaa ccgcgacgcg | 10440 |
| gtcggtccgg ccgagaattt cttcgcgctg ggcggccatt cgttgttggc gacgcgcctg | 10500 |
| gtatcgcgca tccgcgcagc ctgcggcgtg cagttgccgc tgcgcgccgt gttcgaggaa | 10560 |
| cccacgccgg cggcgctggc gcggctggtg gaacgggccg gcggcgacaa cgccgggccg | 10620 |
| gcgccgcgcg aacgctcggg ctggcatccg ctcagctcgc agcagcagcg cctgtggttc | 10680 |
| ctcgaccgct tcgagcccgc caacccgttc tacaacatcc cgctcgcgct gcgcctgcgc | 10740 |
| ggcacgttgg tgccggcgca gctgcagcaa agcctcgatg cgctggccgc gcgccatccg | 10800 |
| tccctgcgca cccgcttcgc cacgcaggac ggccagccgg tacaggaaat cctggcaccg | 10860 |
| gcagcggtgc cgctggcgct cacggacctg acgggactgg ctccggcgca gcgcgaggag | 10920 |
| gcggcccggc gcgccgccgc caccgtgacg ctgcagccgt tcgtgctgga acagggcaat | 10980 |
| ctgctgcgtg cggcgctgct gcggctggac gatgccgacc atgtgctggt actggtggtc | 11040 |
| atcacatcg tcagcgatgg ccgtmwcgct ggcggtgctc gccgacgaac tcgcggcgtg | 11100 |
| taccgcgccg gcacgaccgg cggcgccgcg gcgctgccgc cgctgccatt gcactacagc | 11160 |
| gatttcgcgc actggcagcg cgactggctg cagcagccgg ccgcgctgcg ccagctggcc | 11220 |
| tactggaacg ctcaactggc cgacgcgccg gccgtgcacg cgctgccgct ggaccggccg | 11280 |
| cgcccggcca tccagagcta tcgcggcgcg acgcacggtt tcgccatcgg cgccgcgacg | 11340 |
| ctggccgggc tgcgtgagct ggcagccgcg caggcggaac cgaccacgct gttcatggtg | 11400 |
| ctgtgcgccg ccttcaatgt gctgctgtac cgtcacagcg gccaggccga cctgtgcatc | 11460 |
| ggtaccccga tcgccaaccg ccagcacgac ggcctggacc gggtggtggg cttctttgcc | 11520 |
| aacacgctgg tgctgcgcag ccggccggct cccggccagc cgttccagca gttcctgcgc | 11580 |
| gacgttcgcg cgacggcgct ggacgcctac gccaaccagg acatcgcctt cgaacgcgtg | 11640 |
| gtggaggcgg tcaagccgca acgtcatacc agccatgcgc cgctgttcca ggtcatgctc | 11700 |
| tccctgcagg agtcgctggc cctgccgcag gtggacgata cgctgcggct ggaagcgctc | 11760 |
| acgctggaca gttccgtggc gcgcttcgac ctgacgctca gcctggtgga ggaaggcggc | 11820 |
| acgctgctgg cggcgttcga gtacaacacc gacctgttcg acgccgcgac catcgagcgc | 11880 |
| tgggccggcc acttcagcca cctgctcgat gcggtggtgg ccacgccgca gctggcgctg | 11940 |
| gatcgcctgc cgttgctgga cgacgccgag cgtcgtgacg tactgctggc cagcgccggc | 12000 |
| gagcgcgccg gcccggtcgg cgacaccgtg ctgcatgcgc tgttcgaaca gcaggcgctg | 12060 |
| cgcatccgc agcgttgcgc ggcgcaggcc ggggccgcca gcatcaccta tggtgagctc | 12120 |
| aatacgcgtg ccgccgagct ggcattgcgg ctgcgccacg ccggggtcgc agcgggcgac | 12180 |
| cgggtggcgg tgcacgcgca gcgctcgctc gagctgctgg tcgcgctgct cggcgtgctg | 12240 |
| aaggccggtg ccgcctacgt gccgctcgat ccggcacagc cgcaggaacg gctcgctcat | 12300 |
| atgctgcgcg acagtgcgcc ggccgccgtg ctgacccagc aggggctggc cggtggcgcg | 12360 |
| ctgctggcaa gtgtcccgtg ccgtgtgttg ttactggacg ggccagccgc cgccgcaccc | 12420 |
| gcgccgctgg cggacgtgct cgtacaaccg cacgacctgg cgtatgtgat gtacacgtcc | 12480 |
| ggttcgacgg gtatgccgaa gggcgtgatg gtcgaacatg ccagcatcgt caacacggtg | 12540 |
| cgcgcgcatg tgcggcaatg cgcgctgcag gcccaggatc gcgtgctgca gtttgtctcg | 12600 |
| tacggcttcg acgtctcggc cggcgagatc ttcggcgcgt tcgcggccgg cgccacgctc | 12660 |
| gtgctgcggc cggacgagct gcgcgtgccg gacgaagcgt tcgccgcctt cctgcgcgag | 12720 |
| caggccgtta ccgtggccga cctgccggcg gcgttctggc accagtgggt gcacgagatc | 12780 |
| gccgccggcc gcagcttgcc ggggccggcg ttgcggctcg tcctggccgg cggcgaaaag | 12840 |
| gccgacgtgg cgcgcctgcg cacctggctg accctgccgg caacgcggca cgtacgctgg | 12900 |
| atcaatgcct atggccccac cgagaccacg gtcaacgcga gttacatgcc gtatgacgcg | 12960 |
| ctgtccgagc cgccagccgg cgaggtgccg atcggccggc cgatcgacaa taccgtcgcg | 13020 |
| tatgtcctcg acgcacacct gcagccggta gccttcggta tcgccggcga gatctacctc | 13080 |
| ggcggcgctg gcgtggcgcg cggctacctg aaccagccgg aactgaccga acgcgcgttt | 13140 |
| gtcgccgatc cgttcgccgg cggcgcggcg cgcatgtacc gctccggcga cctgggacgc | 13200 |
| cggctggacg acggtacgct cgaatacctg ggccgtaacg acagccaggt gaaattgcgc | 13260 |
| ggctaccgca tcgagctggg cgaaatccag tcgcgcctgg ccacgctgga cggcgtgcgc | 13320 |
| gaggcatgcg tcatgctgcg cgaggtggcc ggcacaccgc gcctggtggc ttacctggcg | 13380 |
| gcggcggagg gcatgcagct gtccgctgcg gagctgcgtc gcatgctggc cgccagcctg | 13440 |
| ccggactata tggtgccgtc ggccttcgtc tggctgccgg tcctgccggt caatgccagt | 13500 |
| ggcaaggtcg agacggcggc gttgccggaa ccggggcccg ccgacatgga agcgcgcgtg | 13560 |
| atcgaaacgc cggtgggagc gcgcgagcag ctgctggcgc agatctggca ggacttgctg | 13620 |
| gcattgccgc aggtgagccg gcaggatcac ttcttcgaac tgggcggcca ctcgctgatg | 13680 |
| gtggtgacct tgatcgaccg actgcatcaa cacgacctgc atgtggacgt gcgtaccgta | 13740 |
| ttttccagcc cgacgctggc ggcgatggcg gcggccctgg ccgaccgcgc cggcgcgacg | 13800 |
| gccgcctttg tcgcaccacc gaacctgatt ccgggcgaat ttgccgcctc ggcctccacc | 13860 |
| gatcaagcca actttgaaga gtttgaacta tga | 13893 |
| (Protein Sequence of eppB) | |
| SEQ ID NO 6 | |
| Val Glu Leu Gly Glu Pro Leu Pro Ala Leu Asn Thr Met Ile Thr | |
| 2150 2155 2160 | |
| Ala Gly Glu Gln Leu Arg Ile Ser Pro Ala Ile Val Gln Phe Phe | |
| 2165 2170 2175 | |
| Arg Met Arg Ala Gly Arg Ser Leu His Asn Tyr Tyr Gly Pro Thr | |
| 2180 2185 2190 | |
| Glu Ser His Val Val Thr Thr Tyr Val Leu Asp Gly Asp Pro Gly | |
| 2195 2200 2205 | |
| Ala Trp Pro Ala Leu Pro Pro Ile Gly Ala Pro Ile Ala Asn Thr | |
| 2210 2215 2220 | |
| Gln Ile Tyr Ile Leu Asp Ala Ala Leu Gln Pro Val Ala Leu Gly | |
| 2225 2230 2235 | |
| Ala His Gly Glu Leu Tyr Ile Ala Gly Asp Cys Leu Ala Asp Gly | |
| 2240 2245 2250 | |
| Tyr Leu Asn Arg Pro Asp Leu Thr Ala Glu Arg Phe Val Gly Asn | |
| 2255 2260 2265 | |
| Val Phe Arg Pro Gly Thr Arg Met Tyr Lys Thr Gly Asp Ile Ala | |
| 2270 2275 2280 | |
| Arg Trp Leu Glu Asp Gly Asn Ile Glu Tyr Leu Gly Arg Asn Asp | |
| 2285 2290 2295 | |
| Ser Gln Val Lys Ile Arg Gly Tyr Arg Ile Glu Pro Gly Glu Val | |
| 2300 2305 2310 | |
| Glu Ala Ala Leu Ala Ala Cys Ala Gly Val Arg Glu Ala Val Val | |
| 2315 2320 2325 | |
| Val Ala Arg Glu Asp Val Pro Gly Gln Lys Arg Leu Val Ala Tyr | |
| 2330 2335 2340 | |
| Leu Leu Ala Gln Pro Gly His Thr Leu Ala Pro Ala Ala Leu Arg | |
| 2345 2350 2355 | |
| Asp Arg Leu Ala Thr Val Leu Pro Asp Tyr Met Val Pro Ala Ala | |
| 2360 2365 2370 | |
| Phe Val Cys Met Thr Ala Phe Pro Val Ser Pro Asn Gly Lys Leu | |
| 2375 2380 2385 | |
| Asp Arg Arg Ala Leu Pro Ala Pro Asp Ala Ala Ala Gln Leu Arg | |
| 2390 2395 2400 | |
| Gln Pro Tyr Glu Ala Pro Gln Gly Ser Thr Glu Thr Ala Leu Ala | |
| 2405 2410 2415 | |
| Ala Ile Trp Glu Asp Leu Leu Ala Val Arg Asp Val Gly Arg Arg | |
| 2420 2425 2430 | |
| Asp His Phe Phe Glu Leu Gly Gly His Ser Leu Leu Ala Val Arg | |
| 2435 2440 2445 | |
| Leu Thr Thr Arg Val Arg Gln Val Leu Gln Arg Glu Leu Ala Leu | |
| 2450 2455 2460 | |
| Arg Ala Leu Phe Glu Gln Pro Val Leu Ala Asp Leu Ala Arg Val | |
| 2465 2470 2475 | |
| Val Asp Gly Leu Asp Ser Ala Gly Thr Ala Pro Leu Arg Ala Leu | |
| 2480 2485 2490 | |
| Pro Arg Thr Pro Asp Gln Val Leu Pro Leu Ser Phe Ala Gln Gln | |
| 2495 2500 2505 | |
| Arg Leu Trp Phe Val Gln Glu Leu Glu Gly Pro Thr Pro Thr Tyr | |
| 2510 2515 2520 | |
| Asn Met Pro Ala Ala Leu Arg Leu Thr Gly Arg Leu Asp Ala Ala | |
| 2525 2530 2535 | |
| Ala Leu Glu Pro Ala Leu Gln Tyr Leu Ile Glu Arg His Glu Val | |
| 2540 2545 2550 | |
| Leu Arg Thr Asn Phe Asp Ser Val Glu Gly Val Pro His Leu Arg | |
| 2555 2560 2565 | |
| Ile Ala Pro Ser Arg Thr Val Thr Leu Ala Val Thr Asp Val Ala | |
| 2570 2575 2580 | |
| Pro Asp Glu Val Glu Ala Arg Ala Ala Arg His Ala Ala Leu Pro | |
| 2585 2590 2595 | |
| Phe Asp Leu Ala Arg Glu Pro Leu Leu Arg Ala Glu Leu Leu Arg | |
| 2600 2605 2610 | |
| Leu Ser Ala Asp Gln His Val Leu Leu Leu Asn Val His His Ile | |
| 2615 2620 2625 | |
| Val Ser Asp Gly Trp Ser Leu Asn Ile Leu Ala Asp Glu Trp Leu | |
| 2630 2635 2640 | |
| Arg Ala Tyr Asp Ala Leu Arg Ala Gly Arg Ala Pro Ala Leu Pro | |
| 2645 2650 2655 | |
| Val Leu Pro Leu Gln Tyr Ala Asp Tyr Ala Tyr Trp Gln Arg Glu | |
| 2660 2665 2670 | |
| Gln Leu Thr Glu Ala Val Arg Glu Arg Gln Leu Ala Tyr Trp Thr | |
| 2675 2680 2685 | |
| Gly Gln Leu Ala Gly Ala Pro Glu Leu Leu Asp Leu Pro Thr Asp | |
| 2690 2695 2700 | |
| Arg Val Arg Pro Ala Val Gln Arg Phe Asp Gly Gly Asp Glu Gln | |
| 2705 2710 2715 | |
| Leu Arg Leu Asp Pro Ala Leu Ser His Ala Val Arg Gln Leu Gly | |
| 2720 2725 2730 | |
| His Ala His Asn Ala Ser Leu Phe Met Thr Leu Val Thr Ala Phe | |
| 2735 2740 2745 | |
| Gly Leu Leu Leu Gly Arg Leu Ser Gly Gln Asp Asp Val Leu Val | |
| 2750 2755 2760 | |
| Gly Val Pro Gln Ala Thr Arg Asp Arg Arg Glu Leu Glu Gly Met | |
| 2765 2770 2775 | |
| Leu Gly Met Leu Leu Gly Asn Leu Val Leu Arg Met Arg Leu Asp | |
| 2780 2785 2790 | |
| Asp Ala Ala Gly Phe Gly Thr Leu Leu Glu Gln Val Arg Arg Thr | |
| 2795 2800 2805 | |
| Ala Leu Glu Ala Tyr Glu His Ser Ala Ile Pro Phe Glu Gln Val | |
| 2810 2815 2820 | |
| Val Asp Ala Leu Pro Leu Gln Arg Asp Leu Ser Arg Asn Pro Leu | |
| 2825 2830 2835 | |
| Phe Gln Val Phe Phe Asn Met Leu Asn Leu Pro Glu Thr Asn Tyr | |
| 2840 2845 2850 | |
| Thr Ser Pro Glu Leu Ala Ile Glu Gly Leu Gln Ser Thr Leu Leu | |
| 2855 2860 2865 | |
| Asp Ala Lys Phe Asp Leu Thr Leu Tyr Ala Gln Asp Ser Glu Glu | |
| 2870 2875 2880 | |
| Gly Ile Leu Leu His Leu Val Tyr Asn Arg Gly Leu Phe Asp Ala | |
| 2885 2890 2895 | |
| Gln Arg Met Arg Glu Leu Leu Arg Gln Tyr His Ser Leu Leu Glu | |
| 2900 2905 2910 | |
| Gln Val Ser Gln Ala Pro Ala Ile Ala Cys Lys Ala Val Ser Leu | |
| 2915 2920 2925 | |
| Leu Thr Ala Pro Ala Arg Ala Val Leu Pro Asp Pro Ala Val Val | |
| 2930 2935 2940 | |
| Leu Asp Ala Thr Trp His Gly Ser Ile Pro Gly Arg Phe Ala Ala | |
| 2945 2950 2955 | |
| Leu Val Ala Ala Gln Pro Ala Ala Leu Ala Val Thr Ala Ala His | |
| 2960 2965 2970 | |
| Leu Gln Trp Thr Tyr Ala Glu Leu Asp Glu Arg Ser Glu Ala Val | |
| 2975 2980 2985 | |
| Ala Cys Trp Leu Gln Glu Ala Gly Val Gly Ala Gly Ala Val Val | |
| 2990 2995 3000 | |
| Ala Ile Cys Ala Ala Arg Arg Ala Ala Leu Val Pro Ala Val Leu | |
| 3005 3010 3015 | |
| Gly Val Leu Lys Ala Gly Ala Ala Tyr Thr Ile Val Asp Pro Ala | |
| 3020 3025 3030 | |
| Tyr Pro Ala Glu His Val Arg Ala Cys Leu Ala Val Ala Arg Pro | |
| 3035 3040 3045 | |
| Ala Ala Trp Leu Thr Val Ala Glu Gly Gly Asp Ala Ala Leu Leu | |
| 3050 3055 3060 | |
| Ala Cys Leu Pro Ala Pro Val Pro Arg Leu Asp Leu Ser Gly Asn | |
| 3065 3070 3075 | |
| Asp Gly Trp Pro Val Leu Ala Ala Gly Val Arg Ala Val Pro Ala | |
| 3080 3085 3090 | |
| Ala Trp Thr Ala Asp Asp Val Ala Val Leu Thr Phe Thr Ser Gly | |
| 3095 3100 3105 | |
| Ser Thr Gly Leu Pro Lys Ala Val Glu Gly Arg His Gly Ala Leu | |
| 3110 3115 3120 | |
| Thr His Phe Tyr Pro Trp Leu Gln Gln His Phe Gly Met Gly Pro | |
| 3125 3130 3135 | |
| Gln Asp Arg Tyr Ala Leu Leu Ser Gly Leu Ala His Asp Pro Leu | |
| 3140 3145 3150 | |
| Gln Arg Asp Ile Phe Asn Thr Leu Trp Met Gly Ala Ser Leu His | |
| 3155 3160 3165 | |
| Val Pro Pro Val Asp Ala Ile Gly Pro Gly Leu Leu Ala Asp Trp | |
| 3170 3175 3180 | |
| Met Ala Ala Glu Asn Ile Ser Ala Val Asn Leu Thr Pro Ala Met | |
| 3185 3190 3195 | |
| Leu Gln Leu Leu Cys Gln Asp Ala Arg Ala Leu Pro Thr Leu Arg | |
| 3200 3205 3210 | |
| His Ala Phe Leu Val Gly Asp Ile Leu Thr Gln Ala Asp Val Ala | |
| 3215 3220 3225 | |
| Leu Leu Gln Gln Val Ala Pro Arg Cys Ala Val Val Ser Tyr Tyr | |
| 3230 3235 3240 | |
| Gly Ala Thr Glu Ala Gln Arg Ala Phe Gly Met Val Glu Ile Ala | |
| 3245 3250 3255 | |
| Pro Gly Thr Ala Ala Gly Leu Thr Arg Asp Val Ile Ala Leu Gly | |
| 3260 3265 3270 | |
| His Gly Ile Pro Gly Val Gln Leu Leu Val Leu Asn Gly Ala Gly | |
| 3275 3280 3285 | |
| Thr Leu Ala Gly Ile Gly Glu Val Gly Glu Val Cys Ile Arg Ser | |
| 3290 3295 3300 | |
| Pro His Leu Ala Arg Gly Tyr Arg Asp Asp Ala Ala Met Thr Ala | |
| 3305 3310 3315 | |
| Arg Gln Phe Val Ala Asn Pro Phe Gly Gly Gly Asp Arg Leu Tyr | |
| 3320 3325 3330 | |
| Arg Thr Gly Asp Leu Gly Arg Tyr Leu Pro Asp Gly Met Val Ala | |
| 3335 3340 3345 | |
| Gly Leu Gly Arg Asn Asp Gln Gln Val Lys Leu Arg Gly Phe Arg | |
| 3350 3355 3360 | |
| Ile Glu Leu Gly His Val Glu Ala Ala Leu Ala Arg Leu Pro Gln | |
| 3365 3370 3375 | |
| Val Arg Glu Ala Val Val Leu Ala Leu Gly Ser Gly Glu Ala Arg | |
| 3380 3385 3390 | |
| Arg Leu Val Ala Tyr Val Val Pro Arg Gly Thr Phe Asp Ala Asp | |
| 3395 3400 3405 | |
| Ala Ala Ala Ala Ala Leu Arg Gly Thr Leu Pro Asp Tyr Met Arg | |
| 3410 3415 3420 | |
| Pro Ala Ala Tyr Val Val Leu Glu Arg Leu Pro Leu Thr Pro Asn | |
| 3425 3430 3435 | |
| Gly Lys Leu Asp Arg Arg Ala Leu Pro Ala Pro Ala Ala Thr Pro | |
| 3440 3445 3450 | |
| Ala Val Ala Asp Thr Ala Pro Ala Thr Ala Leu Glu Ala Ser Leu | |
| 3455 3460 3465 | |
| Cys Ala Leu Met Ala Glu Leu Leu Asn Arg Asp Ala Val Gly Pro | |
| 3470 3475 3480 | |
| Ala Glu Asn Phe Phe Ala Leu Gly Gly His Ser Leu Leu Ala Thr | |
| 3485 3490 3495 | |
| Arg Leu Val Ser Arg Ile Arg Ala Ala Cys Gly Val Gln Leu Pro | |
| 3500 3505 3510 | |
| Leu Arg Ala Val Phe Glu Glu Pro Thr Pro Ala Ala Leu Ala Arg | |
| 3515 3520 3525 | |
| Leu Val Glu Arg Ala Gly Gly Asp Asn Ala Gly Pro Ala Pro Arg | |
| 3530 3535 3540 | |
| Glu Arg Ser Gly Trp His Pro Leu Ser Ser Gln Gln Gln Arg Leu | |
| 3545 3550 3555 | |
| Trp Phe Leu Asp Arg Phe Glu Pro Ala Asn Pro Phe Tyr Asn Ile | |
| 3560 3565 3570 | |
| Pro Leu Ala Leu Arg Leu Arg Gly Thr Leu Val Pro Ala Gln Leu | |
| 3575 3580 3585 | |
| Gln Gln Ser Leu Asp Ala Leu Ala Ala Arg His Pro Ser Leu Arg | |
| 3590 3595 3600 | |
| Thr Arg Phe Ala Thr Gln Asp Gly Gln Pro Val Gln Glu Ile Leu | |
| 3605 3610 3615 | |
| Ala Pro Ala Ala Val Pro Leu Ala Leu Thr Asp Leu Thr Gly Leu | |
| 3620 3625 3630 | |
| Ala Pro Ala Gln Arg Glu Glu Ala Ala Arg Arg Ala Ala Ala Thr | |
| 3635 3640 3645 | |
| Val Thr Leu Gln Pro Phe Val Leu Glu Gln Gly Asn Leu Leu Arg | |
| 3650 3655 3660 | |
| Ala Ala Leu Leu Arg Leu Asp Asp Ala Asp His Val Leu Val Leu | |
| 3665 3670 3675 | |
| Val Val His His Ile Val Ser Asp Gly Arg Ala Gly Gly Ala Arg | |
| 3680 3685 3690 | |
| Arg Arg Thr Arg Gly Val Tyr Arg Ala Gly Thr Thr Gly Gly Ala | |
| 3695 3700 3705 | |
| Ala Ala Leu Pro Pro Leu Pro Leu His Tyr Ser Asp Phe Ala His | |
| 3710 3715 3720 | |
| Trp Gln Arg Asp Trp Leu Gln Gln Pro Ala Ala Leu Arg Gln Leu | |
| 3725 3730 3735 | |
| Ala Tyr Trp Asn Ala Gln Leu Ala Asp Ala Pro Ala Val His Ala | |
| 3740 3745 3750 | |
| Leu Pro Leu Asp Arg Pro Arg Pro Ala Ile Gln Ser Tyr Arg Gly | |
| 3755 3760 3765 | |
| Ala Thr His Gly Phe Ala Ile Gly Ala Ala Thr Leu Ala Gly Leu | |
| 3770 3775 3780 | |
| Arg Glu Leu Ala Ala Ala Gln Ala Glu Pro Thr Thr Leu Phe Met | |
| 3785 3790 3795 | |
| Val Leu Cys Ala Ala Phe Asn Val Leu Leu Tyr Arg His Ser Gly | |
| 3800 3805 3810 | |
| Gln Ala Asp Leu Cys Ile Gly Thr Pro Ile Ala Asn Arg Gln His | |
| 3815 3820 3825 | |
| Asp Gly Leu Asp Arg Val Val Gly Phe Phe Ala Asn Thr Leu Val | |
| 3830 3835 3840 | |
| Leu Arg Ser Arg Pro Ala Pro Gly Gln Pro Phe Gln Gln Phe Leu | |
| 3845 3850 3855 | |
| Arg Asp Val Arg Ala Thr Ala Leu Asp Ala Tyr Ala Asn Gln Asp | |
| 3860 3865 3870 | |
| Ile Ala Phe Glu Arg Val Val Glu Ala Val Lys Pro Gln Arg His | |
| 3875 3880 3885 | |
| Thr Ser His Ala Pro Leu Phe Gln Val Met Leu Ser Leu Gln Glu | |
| 3890 3895 3900 | |
| Ser Leu Ala Leu Pro Gln Val Asp Asp Thr Leu Arg Leu Glu Ala | |
| 3905 3910 3915 | |
| Leu Thr Leu Asp Ser Ser Val Ala Arg Phe Asp Leu Thr Leu Ser | |
| 3920 3925 3930 | |
| Leu Val Glu Glu Gly Gly Thr Leu Leu Ala Ala Phe Glu Tyr Asn | |
| 3935 3940 3945 | |
| Thr Asp Leu Phe Asp Ala Ala Thr Ile Glu Arg Trp Ala Gly His | |
| 3950 3955 3960 | |
| Phe Ser His Leu Leu Asp Ala Val Val Ala Thr Pro Gln Leu Ala | |
| 3965 3970 3975 | |
| Leu Asp Arg Leu Pro Leu Leu Asp Asp Ala Glu Arg Arg Asp Val | |
| 3980 3985 3990 | |
| Leu Leu Ala Ser Ala Gly Glu Arg Ala Gly Pro Val Gly Asp Thr | |
| 3995 4000 4005 | |
| Val Leu His Ala Leu Phe Glu Gln Gln Ala Leu Ala His Pro Gln | |
| 4010 4015 4020 | |
| Arg Cys Ala Ala Gln Ala Gly Ala Ala Ser Ile Thr Tyr Gly Glu | |
| 4025 4030 4035 | |
| Leu Asn Thr Arg Ala Ala Glu Leu Ala Leu Arg Leu Arg His Ala | |
| 4040 4045 4050 | |
| Gly Val Ala Ala Gly Asp Arg Val Ala Val His Ala Gln Arg Ser | |
| 4055 4060 4065 | |
| Leu Glu Leu Leu Val Ala Leu Leu Gly Val Leu Lys Ala Gly Ala | |
| 4070 4075 4080 | |
| Ala Tyr Val Pro Leu Asp Pro Ala Gln Pro Gln Glu Arg Leu Ala | |
| 4085 4090 4095 | |
| His Met Leu Arg Asp Ser Ala Pro Ala Ala Val Leu Thr Gln Gln | |
| 4100 4105 4110 | |
| Gly Leu Ala Gly Gly Ala Leu Leu Ala Ser Val Pro Cys Arg Val | |
| 4115 4120 4125 | |
| Leu Leu Leu Asp Gly Pro Ala Ala Ala Ala Pro Ala Pro Leu Ala | |
| 4130 4135 4140 | |
| Asp Val Leu Val Gln Pro His Asp Leu Ala Tyr Val Met Tyr Thr | |
| 4145 4150 4155 | |
| Ser Gly Ser Thr Gly Met Pro Lys Gly Val Met Val Glu His Ala | |
| 4160 4165 4170 | |
| Ser Ile Val Asn Thr Val Arg Ala His Val Arg Gln Cys Ala Leu | |
| 4175 4180 4185 | |
| Gln Ala Gln Asp Arg Val Leu Gln Phe Val Ser Tyr Gly Phe Asp | |
| 4190 4195 4200 | |
| Val Ser Ala Gly Glu Ile Phe Gly Ala Phe Ala Ala Gly Ala Thr | |
| 4205 4210 4215 | |
| Leu Val Leu Arg Pro Asp Glu Leu Arg Val Pro Asp Glu Ala Phe | |
| 4220 4225 4230 | |
| Ala Ala Phe Leu Arg Glu Gln Ala Val Thr Val Ala Asp Leu Pro | |
| 4235 4240 4245 | |
| Ala Ala Phe Trp His Gln Trp Val His Glu Ile Ala Ala Gly Arg | |
| 4250 4255 4260 | |
| Ser Leu Pro Gly Pro Ala Leu Arg Leu Val Leu Ala Gly Gly Glu | |
| 4265 4270 4275 | |
| Lys Ala Asp Val Ala Arg Leu Arg Thr Trp Leu Thr Leu Pro Ala | |
| 4280 4285 4290 | |
| Thr Arg His Val Arg Trp Ile Asn Ala Tyr Gly Pro Thr Glu Thr | |
| 4295 4300 4305 | |
| Thr Val Asn Ala Ser Tyr Met Pro Tyr Asp Ala Leu Ser Glu Pro | |
| 4310 4315 4320 | |
| Pro Ala Gly Glu Val Pro Ile Gly Arg Pro Ile Asp Asn Thr Val | |
| 4325 4330 4335 | |
| Ala Tyr Val Leu Asp Ala His Leu Gln Pro Val Ala Phe Gly Ile | |
| 4340 4345 4350 | |
| Ala Gly Glu Ile Tyr Leu Gly Gly Ala Gly Val Ala Arg Gly Tyr | |
| 4355 4360 4365 | |
| Leu Asn Gln Pro Glu Leu Thr Glu Arg Ala Phe Val Ala Asp Pro | |
| 4370 4375 4380 | |
| Phe Ala Gly Gly Ala Ala Arg Met Tyr Arg Ser Gly Asp Leu Gly | |
| 4385 4390 4395 | |
| Arg Arg Leu Asp Asp Gly Thr Leu Glu Tyr Leu Gly Arg Asn Asp | |
| 4400 4405 4410 | |
| Ser Gln Val Lys Leu Arg Gly Tyr Arg Ile Glu Leu Gly Glu Ile | |
| 4415 4420 4425 | |
| Gln Ser Arg Leu Ala Thr Leu Asp Gly Val Arg Glu Ala Cys Val | |
| 4430 4435 4440 | |
| Met Leu Arg Glu Val Ala Gly Thr Pro Arg Leu Val Ala Tyr Leu | |
| 4445 4450 4455 | |
| Ala Ala Ala Glu Gly Met Gln Leu Ser Ala Ala Glu Leu Arg Arg | |
| 4460 4465 4470 | |
| Met Leu Ala Ala Ser Leu Pro Asp Tyr Met Val Pro Ser Ala Phe | |
| 4475 4480 4485 | |
| Val Trp Leu Pro Val Leu Pro Val Asn Ala Ser Gly Lys Val Glu | |
| 4490 4495 4500 | |
| Thr Ala Ala Leu Pro Glu Pro Gly Pro Ala Asp Met Glu Ala Arg | |
| 4505 4510 4515 | |
| Val Ile Glu Thr Pro Val Gly Ala Arg Glu Gln Leu Leu Ala Gln | |
| 4520 4525 4530 | |
| Ile Trp Gln Asp Leu Leu Ala Leu Pro Gln Val Ser Arg Gln Asp | |
| 4535 4540 4545 | |
| His Phe Phe Glu Leu Gly Gly His Ser Leu Met Val Val Thr Leu | |
| 4550 4555 4560 | |
| Ile Asp Arg Leu His Gin His Asp Leu His Val Asp Val Arg Thr | |
| 4565 4570 4575 | |
| Val Phe Ser Ser Pro Thr Leu Ala Ala Met Ala Ala Ala Leu Ala | |
| 4580 4585 4590 | |
| Asp Arg Ala Gly Ala Thr Ala Ala Phe Val Ala Pro Pro Asn Leu | |
| 4595 4600 4605 | |
| Ile Pro Gly Glu Phe Ala Ala Ser Ala Ser Thr Asp Gln Ala Asn | |
| 4610 4615 4620 | |
| Phe Glu Glu Phe Glu Leu | |
| 4625 | |
| (Nucleotide Sequence of eppC) | |
| SEQ ID NO 7 | |
| atgacattcc cacagcttct cgcccacctg cgcagccatt ccatccacct gaaggccgag | 60 |
| cagggcaagc tccaggtccg tgccgagaag ggcacggtcg atgccgagct gcgcacccag | 120 |
| ctcgccgccc acaaggaagc gctgctggcg ctgctcgccg gcgacccggc cgcctgtacc | 180 |
| tggaccgcgg cggcgccgcg catcacgccc gagatgctgc cgctggtgca gctgagccag | 240 |
| ggcgaaatcg atacgatcgt tgccgctacc gaaggtggcg cggcggcgat ccaggacatc | 300 |
| tacccgctgt cgccgctgca ggaaggcttc ctgttccacc acctgctgca ggccgagggc | 360 |
| gacgtctacc tggaacgggc gctgatcggc ttcgacagcc gggacaggct cgatgccttc | 420 |
| gtggcggcgc tgcagaaggt catcgaccgc cacgacatcc tgcgcagcag cgcgcgctgg | 480 |
| caggacctgt cgcgccaggt gcaggtggtg caccggcagg cgcgcctgcc ggtggtcgaa | 540 |
| ctgaagctgc ctgaaggcgg cgacggcatg gccgtgctga aggaagcgac cgatccgcgc | 600 |
| aagctgcgcc tggacctgca ggccgcaccg ctgctggcga cacgcatcgt gccggacggc | 660 |
| gccagcggcg gctggctgat ggcgctgctg cateaccata tggtgtgcga tcacgtgacg | 720 |
| ctcgaattca tcgtcggcga ggtcgcgctg atcctgggcg ggcgcgaggc gctgctgccg | 780 |
| ccggcactgc cgtaccggaa cttcatcgcg cagacgctgg cggtaccggc cagcgcgcac | 840 |
| gagggctact tcaagtcccg ccttgccgat gtgacggaaa ccaccgcgcc gttcggcgtg | 900 |
| ctgaacgtga tgggcgaggg cggcgaagtc agcgagggac acgtgcggct cgatggcgcg | 960 |
| ctggcccagc ggatccgcac gcaggcggcg cgcttcggcg tcactaccgc cgtcctgttc | 1020 |
| cacgtggcat gggcgcgcgt ggttgccctg tgcagcggcc gcgacgacgt cgtattcggt | 1080 |
| accgtgctgt ccggccgcct gcagggcagc gaagccgccg ggcgggtgct gggactgttc | 1140 |
| atcaacgcgc tgccgatccg cctcacgctg gccggacgca gtaccgaaca actggtgcgc | 1200 |
| gaaacctacg ccgacctgac cgcgttgctg gagcacgaac aggcgtcgtt gacgctggca | 1260 |
| caacaatgca gcggtatcgc ggcaccggcg ccgctgttca ccagcctgct caattatcgc | 1320 |
| cacagccacg gcggcgcact gcaggccgac ggccagtggg acggcatgcg cctgctcgat | 1380 |
| ttcggcgaac gcacgaacta tccgatcacc gtttccatcg acgacaccgg cgatggcttt | 1440 |
| gaactggagg cgcagtgcgt gaccgggatc gatcccgcgc gcatcgtgga ctacctggcg | 1500 |
| accgccttgg ccggcctggc cgatggcctg gcgggcggca aggccgccac cgagatggcg | 1560 |
| gtgttgccgg acgccgaacg gacccgcctg ctggagctga gccaaggcgg cccggcttat | 1620 |
| ggcgcggggc tgctgccggc cgaactgctg gcggcgcgct ggccgcagga tgccgccgcg | 1680 |
| atcgccgtca tcgatggcga gcgccacacg agctatgcgg agctggccgc attgagcaac | 1740 |
| cgcctggcgc agcagatgct ggcggccggc gccggacccg gcacccgcgt gggcgtcttc | 1800 |
| gccgagcgcg gactggcgat ggtcgtggcg ctgctcgcgg tcgtcaaggc gggcgccacc | 1860 |
| tatctgccgc tcgacaccgc gcacccggcc gaccgcctcg gccacatcct gaacgacagc | 1920 |
| gcccctgccg ccgtgatcct gcaggcaggg ctggagacgg cgctgccgcg gcacccggcg | 1980 |
| accgccatcg tgctcgatgc cgatggcatc gcgcgcggac tgccggcggc cccggaaagt | 2040 |
| gcgcccgacc tgcgcgcgct gggcgtaacg ccggccgacg cggcgtacgt catctacact | 2100 |
| tccggttcca ccgggctgcc gaaaggcgtt gccaattcgg gcgccggcct ggtgaaccgc | 2160 |
| ctggactggt tcgccaccga agtgctggat cacgtgccgg tcacggcgat gcgcaccagt | 2220 |
| atcagcttcg tcgactccgt caccgaagtc ctcgatacgc tgctggcggg cggcacgctg | 2280 |
| gtcgtcttcg acaaggccgc cacgctcgac ccggcgacct tcgcggaagg cacggcgcgc | 2340 |
| tatggcatct cccatctgat ggtggtgccg gcgctgctgc atcacgtgct ggaggtcgcg | 2400 |
| ccgtccgcgc tggcacgcgt gcgcaccgtg atcaccagcg gcgagcggct gccgccggaa | 2460 |
| ctggcgcagc gcctgaaggc cgccttcccg gccatccggc tggtgaacac gtacggctgc | 2520 |
| tccgaagtga acggtgacgc caccgcctgc gattgcgacg gcacggaagc gacggcaacc | 2580 |
| tccgtgatcg gccgtccgat cgcgggcgtg caggcgctgg tgctcgatgg tgcgcgccag | 2640 |
| ctggtaccgc tgggcgctac cggcgagatc tacctcggcg gcgtgggcgt ggcgggcggc | 2700 |
| tacctcaatc gtccggaatt gacggccgag cgcttcgtgc cgaaccccta cggtgcgggc | 2760 |
| ctgctgtaca agacgggcga cctggggcgc ctgcgcgccg acggcagcct ggaatacctg | 2820 |
| ggccgcaacg acttccaggt caagatgcgc ggcttccgca tcgaactggg cgaaatcgaa | 2880 |
| gcgcggctgc gcacccaccc tggcgtcagc gatgccgtcg tggtcgcgcg cacggagcgg | 2940 |
| gccggcgacc cgcgcctggc cgcgtacgtg ctgccgcgcc gcgagcgcgc cgcggcggcc | 3000 |
| gacgaggccg ggttcagcct gttctatrtc ggtgccacga cctccggagc gggggccgac | 3060 |
| aaataccggc tgtacctgga agcggcccgc ttcgccgacg acaacggctt cgaagccatc | 3120 |
| tggacacccg aacgccactt cgacgatgtg gctggcctgt atcccaaccc tgcgttgctg | 3180 |
| agcgccgcgc tggcgaccag cacgcgccgc gtgcacctgc gcgccggcag cgtggtgctg | 3240 |
| ccgctgcagc agccgatccg ggtggtcgag gactggtcgg tgctggacaa cctgaccggc | 3300 |
| gggcgggtcg gcgtcgcgat cgcctccggc tggcacatgc gcgacttcgt gctggcgccc | 3360 |
| gagcatcacg cgcagcgcca ccgcatcatg tacgaaggca tcgagaccgt gcgcgacctg | 3420 |
| tggcgcggca ctgcgcgttc gttccgcgac ggcgccggcc tgcagagcga aatccaggtc | 3480 |
| tatccacgcc cggtgcaggc cgagctgccg atgtggctga cgtcggccgg cgccaacgag | 3540 |
| accttcatcg aggctggccg gctgggactg aacctgctga cccacctgct gggccagacc | 3600 |
| atccaggaag tggccggcaa gatcgccctg taccgcgaat cgctgcagcg gcacggcttc | 3660 |
| gatccggaca gccgcaaggt cacgttgatg atccacacct acgtcggggc ggaccaggcg | 3720 |
| gctgccctgg cgcaggcacg cgagccgttc aagcgttaca tgaaggcgca cgtggggctg | 3780 |
| ctcaaatcgc tgtcggccac gctgacgcac gcggtcgaca acgtcgaaca ggaaaacctc | 3840 |
| gacagcctgg ccgagcacgc gttccagcgt tatgcgagca gcgcggcctt catcggctcg | 3900 |
| cccgagtcgt gcctgccgat ctatcggcag ttgcgcgagg cgggcgtcga cgaattcgcc | 3960 |
| tgcctgttcg actggatggc gccggaagaa gcgctggccg gactgccgca gttgcgccgg | 4020 |
| ctgcaggacc tggcgcgcag cgatgccccg ggcgtgcgcc agctgcgccg ccacctgttg | 4080 |
| gccgcgctgc ccgattacat ggtgccctcg acgttcagct acttggagcg gatgccgctg | 4140 |
| accgccagcg gcaaggtcaa ccgcctggcc ctgccggcgc ccgagcagca aagtacggaa | 4200 |
| cagacggcct tcgatgcgcc gcagggcgtc gaggagacct ccgtggcacg cctgtggcag | 4260 |
| gacatgctga acgttccgcc gatcgaccgc aacggcaact tcttcgagtt gggcggccac | 4320 |
| tccctgctgg ccgtgcagat gatcgccgcc gtgggcaagc tgttcgccac ggaggtgccg | 4380 |
| ctgcggcagc tgttcgccaa tccgaccgtc gccaaattcg ccgccgcgat tcgcgaacag | 4440 |
| tcgagcaatg cgaagcatcc gaacctggtc acgttgcgca agcgcggcag caaggcgccg | 4500 |
| ctgttcctgg tgcaccccgg cgaaggcgag atcggctacg cgcgcaatct ggcaccccat | 4560 |
| atcgccagcg acgtgccgct gtacggtttc gccgccaccg gcctcctgag cggcgaagcg | 4620 |
| ccgttgacgt cgatcgagga gatcgccagc cgctacgtgc gcgccatgcg ctcggtccag | 4680 |
| ccggaaggtc cgtaccgcat cgccggctgg tcggccggcg gcacgatcgc ctacgagatg | 4740 |
| gcccgtcagt tgctcggcgt ggaccagcag gtcgggttca tcggcctgct cgacaccgac | 4800 |
| ttcagctacg accacctgtt tgcccggacc gatggcgagg aggacctggc gttcgacgag | 4860 |
| atcaactcgc tgctcggtta cctgccaccg cggctgccgg ccgaggtcag cggggaagtg | 4920 |
| cgcctgctgg cgcagagccg cgacttcgat gcgctgctgg cgcgcatgca tgcgcacgat | 4980 |
| ttcatcccga aaggcgtcga tggcggcatc ctgcagcgcc acctcgccct gcgccatgcc | 5040 |
| ctggccgtgg cgctgtatcg ctatcagccg cagcgcctgc cgatcggcgt gacgctgttc | 5100 |
| tcggccagcg gcgaaagccg cgtcgacccg acgatcggct ggcgcgcgca ccacgcggcc | 5160 |
| gacctgctgc acctgatccc ggtcagcggc acgcactata cgatcgtcga ggagccgaac | 5220 |
| gtcatcgagc tgggcaaggc catcagcgcg gagctggccc gcagccagcc gaacggtccg | 5280 |
| gcaccgtacg cgccgcgcgt cgtcatccag agcggcatgg ccggcgaggc accgctgttc | 5340 |
| tgcgtgccgg gcgcgggcgc cagcgtctcg tcactgcacg aactggccca ggcgctgggc | 5400 |
| gagaacgtgc cggtccatgg cctgcaggcg cgcggcctgg acggcaccat gctgccgcat | 5460 |
| gccgacgtgc agtcggccgc gcgggcctat ctggccgccg tgcgcgacgt gcagccggcc | 5520 |
| gggccatacc ggctgctggg ccactcgttc ggcggctgga tcgctttcga gatggcgcag | 5580 |
| caactgacgg cggccggtga gacggtggag cagctggtcg tcatcgacag ccgcagcccg | 5640 |
| gcgccggaag gcacggcggt gcggcactac acccggatcg agacgctgct ggaactggtg | 5700 |
| gctctgtaca acctgcgcct ggccgacaag ctggccctga cggcggccga cttccggccg | 5760 |
| ctcaacccgg cggcgcaact ggccctgctg cacgagcacc tggtgcgcgc cggcctggtg | 5820 |
| tcgccgcggg cccaaccggg catgctggag ggcgtggtga acgtgctgca ggcgaacctg | 5880 |
| tcgacggtgt accggccagc cagggtgtat gaaggtgccc tgttgctggt caacgccagc | 5940 |
| gagcaggaag ggcgcggcga caatgccgcg cgggtggcgg cctggcgcag ccacgcgccg | 6000 |
| gcgctggtcg aggccgaggc gcctggcaat cacctgacgc tgctggcgtc gccgcacgtg | 6060 |
| gacgcggtgg ccagccgcat cctgggccag gtgccgagca tgctttga | 6108 |

```
(Protein Sequence of eppC)
SEQ ID NO 8
Met Thr Phe Pro Gln Leu Leu Ala His Leu Arg Ser His Ser Ile His
1 5 10 15
Leu Lys Ala Glu Gln Gly Lys Leu Gln Val Arg Ala Glu Lys Gly Thr
            20 25 30
Val Asp Ala Glu Leu Arg Thr Gln Leu Ala Ala His Lys Glu Ala Leu
        35 40 45
Leu Ala Leu Leu Ala Gly Asp Pro Ala Ala Cys Thr Trp Thr Ala Ala
    50 55 60
Ala Pro Arg Ile Thr Pro Glu Met Leu Pro Leu Val Gln Leu Ser Gln
65 70 75 80
Gly Glu Ile Asp Thr Ile Val Ala Ala Thr Glu Gly Gly Ala Ala Ala
                85 90 95
Ile Gln Asp Ile Tyr Pro Leu Ser Pro Leu Gln Glu Gly Phe Leu Phe
            100 105 110
His His Leu Leu Gln Ala Glu Gly Asp Val Tyr Leu Glu Arg Ala Leu
        115 120 125
Ile Gly Phe Asp Ser Arg Asp Arg Leu Asp Ala Phe Val Ala Ala Leu
    130 135 140
Gln Lys Val Ile Asp Arg His Asp Ile Leu Arg Ser Ser Ala Arg Trp
145 150 155 160
Gln Asp Leu Ser Arg Gln Val Gln Val Val His Arg Gln Ala Arg Leu
                165 170 175
Pro Val Val Glu Leu Lys Leu Pro Glu Gly Gly Asp Gly Met Ala Val
            180 185 190
Leu Lys Glu Ala Thr Asp Pro Arg Lys Leu Arg Leu Asp Leu Gln Ala
        195 200 205
Ala Pro Leu Leu Ala Thr Arg Ile Val Pro Asp Gly Ala Ser Gly Gly
    210 215 220
Trp Leu Met Ala Leu Leu His His His Met Val Cys Asp His Val Thr
225 230 235 240
Leu Glu Phe Ile Val Gly Glu Val Ala Leu Ile Leu Gly Gly Arg Glu
                245 250 255
Ala Leu Leu Pro Pro Ala Leu Pro Tyr Arg Asn Phe Ile Ala Gln Thr
            260 265 270
Leu Ala Val Pro Ala Ser Ala His Glu Gly Tyr Phe Lys Ser Arg Leu
        275 280 285
Ala Asp Val Thr Glu Thr Thr Ala Pro Phe Gly Val Leu Asn Val Met
    290 295 300
Gly Glu Gly Gly Glu Val Ser Glu Gly His Val Arg Leu Asp Gly Ala
305 310 315 320
Leu Ala Gln Arg Ile Arg Thr Gln Ala Ala Arg Phe Gly Val Thr Thr
                325 330 335
Ala Val Leu Phe His Val Ala Trp Ala Arg Val Val Ala Leu Cys Ser
            340 345 350
Gly Arg Asp Asp Val Val Phe Gly Thr Val Leu Ser Gly Arg Leu Gln
        355 360 365
Gly Ser Glu Ala Ala Gly Arg Val Leu Gly Leu Phe Ile Asn Ala Leu
    370 375 380
Pro Ile Arg Leu Thr Leu Ala Gly Arg Ser Thr Glu Gln Leu Val Arg
385 390 395 400
Glu Thr Tyr Ala Asp Leu Thr Ala Leu Leu Glu His Glu Gln Ala Ser
                405 410 415
Leu Thr Leu Ala Gln Gln Cys Ser Gly Ile Ala Ala Pro Ala Pro Leu
            420 425 430
Phe Thr Ser Leu Leu Asn Tyr Arg His Ser His Gly Gly Ala Leu Gln
        435 440 445
Ala Asp Gly Gln Trp Asp Gly Met Arg Leu Leu Asp Phe Gly Glu Arg
    450 455 460
Thr Asn Tyr Pro Ile Thr Val Ser Ile Asp Asp Thr Gly Asp Gly Phe
465 470 475 480
Glu Leu Glu Ala Gln Cys Val Thr Gly Ile Asp Pro Ala Arg Ile Val
                485 490 495
Asp Tyr Leu Ala Thr Ala Leu Ala Gly Leu Ala Asp Gly Leu Ala Gly
            500 505 510
Gly Lys Ala Ala Thr Glu Met Ala Val Leu Pro Asp Ala Glu Arg Thr
        515 520 525
Arg Leu Leu Glu Leu Ser Gln Gly Gly Pro Ala Tyr Gly Ala Gly Leu
    530 535 540
Leu Pro Ala Glu Leu Leu Ala Ala Arg Trp Pro Gln Asp Ala Ala Ala
545 550 555 560
Ile Ala Val Ile Asp Gly Glu Arg His Thr Ser Tyr Ala Glu Leu Ala
                565 570 575
Ala Leu Ser Asn Arg Leu Ala Gln Gln Met Leu Ala Ala Gly Ala Gly
            580 585 590
Pro Gly Thr Arg Val Gly Val Phe Ala Glu Arg Gly Leu Ala Met Val
        595 600 605
Val Ala Leu Leu Ala Val Lys Ala Gly Ala Thr Tyr Leu Pro Leu
    610 615 620
Asp Thr Ala His Pro Ala Asp Arg Leu Gly His Ile Leu Asn Asp Ser
625 630 635 640
Ala Pro Ala Ala Val Ile Leu Gln Ala Gly Leu Glu Thr Ala Leu Pro
                645 650 655
Arg His Pro Ala Thr Ala Ile Val Leu Asp Ala Asp Gly Ile Ala Arg
            660 665 670
Gly Leu Pro Ala Ala Pro Glu Ser Ala Pro Asp Leu Arg Ala Leu Gly
        675 680 685
Val Thr Pro Ala Asp Ala Ala Tyr Val Ile Tyr Thr Ser Gly Ser Thr
    690 695 700
Gly Leu Pro Lys Gly Val Ala Asn Ser Gly Ala Gly Leu Val Asn Arg
705 710 715 720
Leu Asp Trp Phe Ala Thr Glu Val Leu Asp His Val Pro Val Thr Ala
                725 730 735
Met Arg Thr Ser Ile Ser Phe Val Asp Ser Val Thr Glu Val Leu Asp
            740 745 750
Thr Leu Leu Ala Gly Gly Thr Leu Val Val Phe Asp Lys Ala Ala Thr
        755 760 765
Leu Asp Pro Ala Thr Phe Ala Glu Gly Thr Ala Arg Tyr Gly Ile Ser
    770 775 780
His Leu Met Val Val Pro Ala Leu Leu His His Val Leu Glu Val Ala
785 790 795 800
Pro Ser Ala Leu Ala Arg Val Arg Thr Val Ile Thr Ser Gly Glu Arg
                805 810 815
Leu Pro Pro Glu Leu Ala Gln Arg Leu Lys Ala Ala Phe Pro Ala Ile
            820 825 830
Arg Leu Val Asn Thr Tyr Gly Cys Ser Glu Val Asn Gly Asp Ala Thr
        835 840 845
Ala Cys Asp Cys Asp Gly Thr Glu Ala Thr Ala Thr Ser Val Ile Gly
    850 855 860
Arg Pro Ile Ala Gly Val Gln Ala Leu Val Leu Asp Gly Ala Arg Gln
865 870 875 880
Leu Val Pro Leu Gly Ala Thr Gly Glu Ile Tyr Leu Gly Gly Val Gly
                885 890 895
Val Ala Gly Gly Tyr Leu Asn Arg Pro Glu Leu Thr Ala Glu Arg Phe
            900 905 910
Val Pro Asn Pro Tyr Gly Ala Gly Leu Leu Tyr Lys Thr Gly Asp Leu
        915 920 925
Gly Arg Leu Arg Ala Asp Gly Ser Leu Glu Tyr Leu Gly Arg Asn Asp
    930 935 940
Phe Gln Val Lys Met Arg Gly Phe Arg Ile Glu Leu Gly Glu Ile Glu
945 950 955 960
Ala Arg Leu Arg Thr His Pro Gly Val Ser Asp Ala Val Val Val Ala
                965 970 975
Arg Thr Glu Arg Ala Gly Asp Pro Arg Leu Ala Ala Tyr Val Leu Pro
            980 985 990
Arg Arg Glu Arg Ala Ala Ala Ala Asp Glu Ala Gly Phe Ser Leu Phe
        995 1000 1005
Tyr Phe Gly Ala Thr Thr Ser Gly Ala Gly Ala Asp Lys Tyr Arg
    1010 1015 1020
Leu Tyr Leu Glu Ala Ala Arg Phe Ala Asp Asp Asn Gly Phe Glu
    1025 1030 1035
Ala Ile Trp Thr Pro Glu Arg His Phe Asp Asp Val Ala Gly Leu
    1040 1045 1050
Tyr Pro Asn Pro Ala Leu Leu Ser Ala Ala Leu Ala Thr Ser Thr
    1055 1060 1065
Arg Arg Val His Leu Arg Ala Gly Ser Val Val Leu Pro Leu Gln
    1070 1075 1080
Gln Pro Ile Arg Val Val Glu Asp Trp Ser Val Leu Asp Asn Leu
    1085 1090 1095
Thr Gly Gly Arg Val Gly Val Ala Ile Ala Ser Gly Trp His Met
    1100 1105 1110
Arg Asp Phe Val Leu Ala Pro Glu His His Ala Gln Arg His Arg
    1115 1120 1125
Ile Met Tyr Glu Gly Ile Glu Thr Val Arg Asp Leu Trp Arg Gly
    1130 1135 1140
Thr Ala Arg Ser Phe Arg Asp Gly Ala Gly Leu Gln Ser Glu Ile
    1145 1150 1155
Gln Val Tyr Pro Arg Pro Val Gln Ala Glu Leu Pro Met Trp Leu
    1160 1165 1170
Thr Ser Ala Gly Ala Asn Glu Thr Phe Ile Glu Ala Gly Arg Leu
    1175 1180 1185
Gly Leu Asn Leu Leu Thr His Leu Leu Gly Gln Thr Ile Gln Glu
    1190 1195 1200
Val Ala Gly Lys Ile Ala Leu Tyr Arg Glu Ser Leu Gln Arg His
    1205 1210 1215
Gly Phe Asp Pro Asp Ser Arg Lys Val Thr Leu Met Ile His Thr
    1220 1225 1230
Tyr Val Gly Ala Asp Gln Ala Ala Ala Leu Ala Gln Ala Arg Glu
    1235 1240 1245
Pro Phe Lys Arg Tyr Met Lys Ala His Val Gly Leu Leu Lys Ser
    1250 1255 1260
Leu Ser Ala Thr Leu Thr His Ala Val Asp Asn Val Glu Gln Glu
    1265 1270 1275
Asn Leu Asp Ser Leu Ala Glu His Ala Phe Gln Arg Tyr Ala Ser
    1280 1285 1290
Ser Ala Ala Phe Ile Gly Ser Pro Glu Ser Cys Leu Pro Ile Tyr
    1295 1300 1305
Arg Gln Leu Arg Glu Ala Gly Val Asp Glu Phe Ala Cys Leu Phe
    1310 1315 1320
Asp Trp Met Ala Pro Glu Glu Ala Leu Ala Gly Leu Pro Gln Leu
    1325 1330 1335
Arg Arg Leu Gln Asp Leu Ala Arg Ser Asp Ala Pro Gly Val Arg
    1340 1345 1350
Gln Leu Arg Arg His Leu Leu Ala Ala Leu Pro Asp Tyr Met Val
    1355 1360 1365
Pro Ser Thr Phe Ser Tyr Leu Glu Arg Met Pro Leu Thr Ala Ser
    1370 1375 1380
Gly Lys Val Asn Arg Leu Ala Leu Pro Ala Pro Glu Gln Gln Ser
    1385 1390 1395
Thr Glu Gln Thr Ala Phe Asp Ala Pro Gln Gly Val Glu Glu Thr
    1400 1405 1410
Ser Val Ala Arg Leu Trp Gln Asp Met Leu Asn Val Pro Pro Ile
    1415 1420 1425
Asp Arg Asn Gly Asn Phe Phe Glu Leu Gly Gly His Ser Leu Leu
    1430 1435 1440
Ala Val Gln Met Ile Ala Ala Val Gly Lys Leu Phe Ala Thr Glu
    1445 1450 1455
Val Pro Leu Arg Gln Leu Phe Ala Asn Pro Thr Val Ala Lys Phe
    1460 1465 1470
Ala Ala Ala Ile Arg Glu Gln Ser Ser Asn Ala Lys His Pro Asn
    1475 1480 1485
Leu Val Thr Leu Arg Lys Arg Gly Ser Lys Ala Pro Leu Phe Leu
    1490 1495 1500
Val His Pro Gly Glu Gly Glu Ile Gly Tyr Ala Arg Asn Leu Ala
    1505 1510 1515
Pro His Ile Ala Ser Asp Val Pro Leu Tyr Gly Phe Ala Ala Thr
    1520 1525 1530
Gly Leu Leu Ser Gly Glu Ala Pro Leu Thr Ser Ile Glu Glu Ile
    1535 1540 1545
Ala Ser Arg Tyr Val Arg Ala Met Arg Ser Val Gln Pro Glu Gly
    1550 1555 1560
Pro Tyr Arg Ile Ala Gly Trp Ser Ala Gly Gly Thr Ile Ala Tyr
    1565 1570 1575
Glu Met Ala Arg Gln Leu Leu Gly Val Asp Gln Gln Val Gly Phe
    1580 1585 1590
Ile Gly Leu Leu Asp Thr Asp Phe Ser Tyr Asp His Leu Phe Ala
    1595 1600 1605
Arg Thr Asp Gly Glu Glu Asp Leu Ala Phe Asp Glu Ile Asn Ser
    1610 1615 1620
Leu Leu Gly Tyr Leu Pro Pro Arg Leu Pro Ala Glu Val Ser Gly
    1625 1630 1635
Glu Val Arg Leu Leu Ala Gln Ser Arg Asp Phe Asp Ala Leu Leu
    1640 1645 1650
Ala Arg Met His Ala His Asp Phe Ile Pro Lys Gly Val Asp Gly
    1655 1660 1665
Gly Ile Leu Gln Arg His Leu Ala Leu Arg His Ala Leu Ala Val
    1670 1675 1680
Ala Leu Tyr Arg Tyr Gln Pro Gln Arg Leu Pro Ile Gly Val Thr
    1685 1690 1695
Leu Phe Ser Ala Ser Gly Glu Ser Arg Val Asp Pro Thr Ile Gly
    1700 1705 1710
Trp Arg Ala His His Ala Ala Asp Leu Leu His Leu Ile Pro Val
    1715 1720 1725
Ser Gly Thr His Tyr Thr Ile Val Glu Glu Pro Asn Val Ile Glu
    1730 1735 1740
Leu Gly Lys Ala Ile Ser Ala Glu Leu Ala Arg Ser Gln Pro Asn
    1745 1750 1755
Gly Pro Ala Pro Tyr Ala Pro Arg Val Val Ile Gln Ser Gly Met
    1760 1765 1770
Ala Gly Glu Ala Pro Leu Phe Cys Val Pro Gly Ala Gly Ala Ser
    1775 1780 1785
Val Ser Ser Leu His Glu Leu Ala Gln Ala Leu Gly Glu Asn Val
    1790 1795 1800
Pro Val His Gly Leu Gln Ala Arg Gly Leu Asp Gly Thr Met Leu
    1805 1810 1815
Pro His Ala Asp Val Gln Ser Ala Ala Arg Ala Tyr Leu Ala Ala
    1820 1825 1830
Val Arg Asp Val Gln Pro Ala Gly Pro Tyr Arg Leu Leu Gly His
    1835 1840 1845
Ser Phe Gly Gly Trp Ile Ala Phe Glu Met Ala Gln Gln Leu Thr
    1850 1855 1860
Ala Ala Gly Glu Thr Val Glu Gln Leu Val Val Ile Asp Ser Arg
    1865 1870 1875
Ser Pro Ala Pro Glu Gly Thr Ala Val Arg His Tyr Thr Arg Ile
    1880 1885 1890
Glu Thr Leu Leu Glu Leu Val Ala Leu Tyr Asn Leu Arg Leu Ala
    1895 1900 1905
Asp Lys Leu Ala Leu Thr Ala Ala Asp Phe Arg Pro Leu Asn Pro
    1910 1915 1920
Ala Ala Gln Leu Ala Leu Leu His Glu His Leu Val Arg Ala Gly
    1925 1930 1935
Leu Val Ser Pro Arg Ala Gln Pro Gly Met Leu Glu Gly Val Val
    1940 1945 1950
Asn Val Leu Gln Ala Asn Leu Ser Thr Val Tyr Arg Pro Ala Arg
    1955 1960 1965
Val Tyr Glu Gly Ala Leu Leu Leu Val Asn Ala Ser Glu Gln Glu
    1970 1975 1980
Gly Arg Gly Asp Asn Ala Ala Arg Val Ala Ala Trp Arg Ser His
    1985 1990 1995
Ala Pro Ala Leu Val Glu Ala Glu Ala Pro Gly Asn His Leu Thr
    2000 2005 2010
Leu Leu Ala Ser Pro His Val Asp Ala Val Ala Ser Arg Ile Leu
    2015 2020 2025
Gly Gln Val Pro Ser Met Leu
    2030 2035
```

Following assembly of the sequence derived from the two clones, a comparative analysis with published NRPS gene clusters was carried out to determine the module and domain organization of the deduced (putative) Empedopeptin biosynthetic NRPS complex, and any associated gene sequences. Associated sequences could encode enzymes involved in “tailoring” reactions, such as hydroxylation of the proline and aspartic acid residues in the peptide, or in the regulation of expression or export of the peptide.

The observed module and domain organization of the identified gene is illustrated in FIG. 1.

As illustrated in FIG. 1, the NRPS portion of the empedopeptin biosynthetic gene cluster spans a region of approximately 31 kb and consists of three NRPS genes, eppA, eppB, and eppC. The first two NRPS genes, eppA and eppB, are separated by an about 2.4 kb insert, which contains the open reading frames of a homoserine-O-succinyl-transferase-like enzyme (eppT), and a putative Zn-dependent hydrolase (eppH).

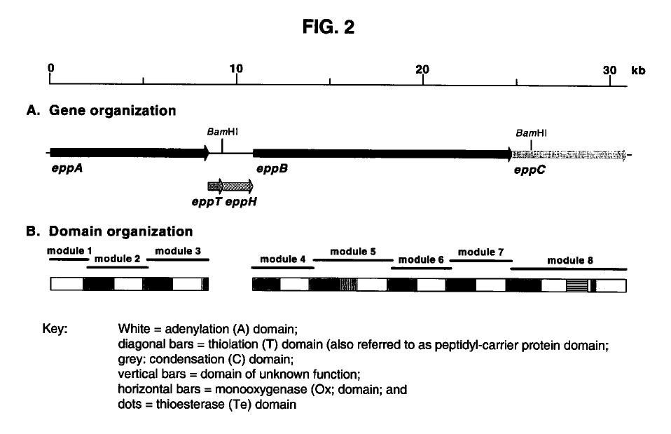

Also as illustrated in FIG. 2, the Epp biosynthetic complex consists of eight modules, of which eppA, eppB, and eppC encodes three, four and one (modules), respectively. Features of the Epp biosynthetic template include: (i) the Epp biosynthetic template starts with an initiation module (domain organization: A-PCP), rather than an elongation module (C-A-PCP); (ii) the coding region of module 5 contains about a 1 kb insert (shown as section with vertical bars), which separates the coding regions of the corresponding C and A domains. The 1 kb-insertion encodes an NRPS catalytic domain that is entirely unique. It has no identifiable homologues in publicly accessible data bases; and (iii) EppC encodes a single (termination) module (module 8). Moreover, the coding region of the adenylation (A) domain in module 8 is disrupted (between core motifs A8 and A9) by about a 1.2 kb insert, encoding a monooxygenase domain.

In FIG. 2, the following key was employed:
White=adenylation (A) domain;
Diagonal bars=thiolation (T) domain (also referred to as peptidyl-carrier protein domain);
Grey=condensation (C) domain;
Vertical bars=domain of unknown function;
Horizontal bars=monooxygenase (Ox) domain; and
Dots=thioesterase (Te) domain.

### VI. FORMULATIONS, ADMINISTRATIONS, AND USES

### A. Pharmaceutically Acceptable Compositions

The present invention includes within its scope pharmaceutically acceptable prodrugs of the compounds of the present invention. A “pharmaceutically acceptable prodrug” means any pharmaceutically acceptable salt, ester, salt of an ester, or other derivative of a compound of the present invention which, upon administration to a recipient, is capable of providing (directly or indirectly) a compound of this invention or an active metabolite or residue thereof. Preferred prodrugs are those that increase the bioavailability of the compounds of this invention when such compounds are administered to a mammal or which enhance delivery of the parent compound to a biological compartment relative to the parent species.

The term “pharmaceutically acceptable carrier, adjuvant, or vehicle” refers to a non-toxic carrier, adjuvant, or vehicle that does not destroy the pharmacological activity of the Empedopeptin with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for parenteral administration or specifically intramuscular injection.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the modulator can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention. As used herein, additional therapeutic agents that are normally administered to treat or prevent a particular disease, or condition, are known as “appropriate for the disease, or condition, being treated.”

In several pharmaceutical compositions comprising Empedopeptin, the carrier is water or saline.

### VII. BIOLOGICAL ACTIVITY

### Materials and Methods

Compounds:

The investigational agent, Empedopeptin, was purified from the culture broth of Empedobacter haloabium strain No. G393-B445 (ATCC 31962) as provided in Konishi, M., Sugawara, K., Hanada, M., Tomita, K., Tomatsu, K., Miyaki, T., and Kawaguchi, H. (1984) Empedopeptin (BMY-28117), a new depsipeptideantibiotic. 1. Production, isolation and properties. J. Antibiot. 37:949-957. The Empedopeptin was stored at −20° C. until the day of the MIC assay. Daptomycin (Lot# CDCX01) was obtained from Cubist, linezolid (Lot# LZD05003) from Pfizer, vancomycin (Lot# 016K1102) from Sigma-Aldrich, and oxacillin (Lot# 1101952) from BioChemika.

The solvent for all of the compounds was deionized water (DIW), and all of the compounds dissolved in the solvent. The stock solutions were allowed to stand in DIW for one hour at room temperature prior to testing to allow time for auto-sterilization. The stock concentration of the test compounds was 5120 μg/mL, resulting in the final test concentration range of 128-0.12 μg/mL.

The test organisms were originally received from clinical sources, or from the American Type Culture Collection. When received, the organisms were sub-cultured onto an appropriate agar medium. Following incubation, colonies were harvested from these plates and cell suspensions prepared and frozen at −80° C. On the day prior to assay, a frozen vial of each culture was thawed and the contents were streaked for isolation onto either Tryptic Soy Agar (Becton Dickinson, Sparks, Md.) or Tryptic Soy Agar (Enhanced Hemolysis; Becton Dickinson) supplemented with 5% sheep blood for streptococci. The agar plates were incubated overnight at 35° C. Staphylococcus aureus ATCC 29213 and Enterococcus faecalis ATCC 29212 were included as quality control isolates in the assay.

Test Medium:

The test medium for the broth microdilution testing was Mueller Hinton II broth (MHB II; BBL# 212322, Lot # 6024003, Becton Dickinson). The broth was prepared at 1.05× normal weight/volume to offset the 5% volume of the drug solution in the final test plates.

For streptococci, lysed horse blood (Lot # H88621; Cleveland Scientific, Bath, Ohio) was added to the MHB II at a final concentration of 2%.

CLSI guidelines recommend that Mueller-Hinton II broth be adjusted to contain 50 mg/L of Ca⁺⁺ ions for proper daptomycin MIC results. Since Mueller-Hinton II broth has already been adjusted by the manufacturer to contain approximately 25 mg/L of Ca⁺⁺ ions, an additional 25 mg/L of Ca⁺⁺ ions was adjusted with 10 mg/mL of CaCl₂.2H₂O (Lot# 084K0215; Sigma-Aldrich) added at a rate of 0.1 mL/L of broth, for each desired increment of 1 mg/L. This supplemented Mueller-Hinton II broth was used only in wells containing daptomycin.

MIC Methodology:

MIC values were determined using a broth microdilution method as recommended by the Clinical and Laboratory Standards Institute (Clinical and Laboratory Standards Institute^{a}. Methods for Dilution Antimicrobial Susceptibility Tests for Bacteria That Grow Aerobically; Approved Standard-Seventh Edition. Clinical and Laboratory Standards Institute document M7-A7 [ISBN 1-56238-587-9]. Clinical and Laboratory Standards Institute, 940 West Valley Road, Suite 1400, Wayne, Pa. 19087-1898 USA, 2006). Automated liquid handlers (Multidrop 384, Labsystems, Helsinki, Finland; Biomek 2000 and Multimek 96, Beckman Coulter, Fullerton Calif.) were used to conduct serial dilutions and make liquid transfers.

Wells of two standard 96-well microdilution plates (Falcon 3918; Becton Dickinson) were filled with 150 μL of DMSO using the Multidrop 384. These plates were used to prepare the drug “mother plates” that provided the serial drug dilutions for replicate “daughter plates”. The Biomek 2000 was used to transfer 150 μl of each stock solution from the wells of column 1 of a deep well plate to the corresponding wells in column 1 of the mother plate and to make eleven 2-fold serial dilutions in the mother plates. The wells of column 12 contained no drug and were the organism growth control wells. Each mother plate has the capacity to create a total of 12 daughter plates.

The daughter plates were loaded with 180 μL of one of the media described above using the Multidrop 384. The wells of the daughter plates ultimately contained 180 μL of MHB II, 10 μL of drug solution, and 10 μL of bacterial inoculum prepared in broth appropriate to the test organism (1.05×). The daughter plates were prepared on the Multimek 96 instrument, which transferred 10 μL of drug solution from each well of the mother plate to each corresponding well of each daughter plate in a single step.

Standardized inoculum of each organism was prepared following Clinical and Laboratory Standards Institute (Clinical and Laboratory Standards Institute^{b}. Performance Standards for Antimicrobial Susceptibility Testing; Sixteenth Informational Supplement. CLSI document M100-S16 [ISBN 1-56238-588-7]. Clinical and Laboratory Standards Institute, 940 West Valley Road, Suite 1400, Wayne, Pa. 19087-1898 USA, 2006) methods. The inoculum for each organism was dispensed into sterile reservoirs divided by length (Beckman Coulter), and the Biomek 2000 was used to inoculate the plates. Daughter plates were placed on the Biomek 2000 work surface in a reversed position so that inoculation occurred from low to high drug concentration. The Biomek 2000 delivered 10 μL of standardized inoculum into each well. This yielded a final cell concentration in the daughter plates of approximately 5×105 colony-forming-units/mL.

Plates were stacked 3 high, covered with a lid on the top plate, placed in plastic bags, and incubated at 35° C. for approximately 20 h. Following incubation, the microplates were removed from the incubator and viewed from the bottom using a plate viewer. An un-inoculated solubility control plate was observed for evidence of drug precipitation. The MIC was read and recorded as the lowest concentration of drug that inhibited visible growth of the organism.

Results:

All of the compounds were soluble in the stock solutions and in the microbiological test media (data not shown). Table 1 details the test organisms and phenotypes and the MIC data for the test agents.

TABLE 1
Minimal Inhibitory Concentration (MIC) Values for Empedopeptin,
Daptomycin, Linezolid, Oxacillin, and Vancomycin

| | Micromyx | | MIC (μg/mL) | | | | |
|---|---|---|---|---|---|---|---|
| Organism | # | Phenotype | Empedopeptin | Daptomycin | Linezolid | Oxacillin | Vancomycin |
| Enterococcus | 101 | VSE¹; | 4 | 1 | 8 | 16 | 4 |
| faecalis | | CLSI² QC³ | | | | | |
| | | strain | | | | | |
| Enterococcus | 413 | ⁴LZD^{R} | 8 | 0.5 | 64 | 64 | 2 |
| faecalis | | from res. | | | | | |
| | | dev. or other | | | | | |
| Enterococcus | 486 | Van⁵ A | 8 | 0.5 | 4 | 16 | >128 |
| faecalis | | | | | | | |
| Enterococcus | 1088 | Van B | 16 | 1 | 4 | 16 | 128 |
| faecalis | | | | | | | |
| Enterococcus | 750 | Van^{S} | 8 | ≦0.12 | 4 | 4 | 1 |
| faecium | | | | | | | |
| Enterococcus | 1721 | ⁶DAP^{R} | 32 | 8 | 2 | 32 | >128 |
| faecium | | | | | | | |
| Enterococcus | 752 | Van A | 16 | 2 | 4 | >128 | >128 |
| faecium | | | | | | | |
| Enterococcus | 1120 | Van B | 16 | 2 | 4 | >128 | 32 |
| faecium | | | | | | | |
| Staphylococcus | 100 | CLSI QC | 4 | 0.5 | 8 | 0.25 | 2 |
| aureus | | strain | | | | | |
| Staphylococcus | 1002 | MSSA⁷, | 4 | 0.5 | 4 | 0.5 | 1 |
| aureus | | macrolide^{R} | | | | | |
| Staphylococcus | 1004 | MRSA⁸, | 8 | 0.25 | 4 | 16 | 1 |
| aureus | | ⁹FA^{R} | | | | | |
| Staphylococcus | 1016 | FA^{R} | 8 | 0.25 | 8 | 32 | 1 |
| aureus | | | | | | | |
| Staphylococcus | 1651 | LZD^{R} | 4 | 0.5 | 64 | 128 | 1 |
| aureus | | clinical | | | | | |
| | | isolate | | | | | |
| Staphylococcus | 1723 | VISA¹⁰ | 4 | 1 | 4 | >128 | 8 |
| aureus | | | | | | | |
| Staphylococcus | 1727 | ¹¹GM^{R} | 8 | 0.25 | 4 | 0.25 | 1 |
| aureus | | | | | | | |
| Staphylococcus | 1730 | Community- | 4 | 0.5 | 8 | 32 | 2 |
| aureus | | acquired | | | | | |
| | | MRSA | | | | | |
| Staphylococcus | 1731 | ¹²CHL^{R} | 4 | 0.25 | 8 | 0.25 | 1 |
| aureus | | | | | | | |
| Staphylococcus | 106 | ¹³RA^{R} | 0.5 | 0.5 | 4 | >128 | 1 |
| aureus | | | | | | | |
| Staphylococcus | 835 | MSSE¹⁴ | 8 | 0.5 | 4 | ≦0.12 | 2 |
| epidermidis | | | | | | | |
| Staphylococcus | 108 | MRSE¹⁵ | 8 | 0.5 | 4 | 64 | 4 |
| epidermidis | | | | | | | |
| Streptococcus | 374 | Wild type | 1 | ≦0.12 | 4 | 16 | 0.5 |
| pneumoniae | | | | | | | |
| Streptococcus | 375 | parC, gyrB | 2 | ≦0.12 | 4 | ≦0.12 | 0.5 |
| pneumoniae | | | | | | | |
| Streptococcus | 376 | parC, gyrA | 2 | ≦0.12 | 4 | ≦0.12 | 0.25 |
| pneumoniae | | | | | | | |
| Streptococcus | 379 | parC, gyrA, | 2 | 0.25 | 4 | ≦0.12 | 0.5 |
| pneumoniae | | gyrB | | | | | |
| Streptococcus | 927 | ¹⁶mef(A) | <0.12 | ≦0.12 | 2 | 16 | 0.5 |
| pneumoniae | | | | | | | |
| Streptococcus | 928 | ¹⁷erm(B) | 0.5 | ≦0.12 | 2 | 16 | 0.5 |
| pneumoniae | | | | | | | |
| Streptococcus | 985 | Susceptible | ≦0.12 | ≦0.12 | 4 | ≦0.12 | 0.5 |
| pyogenes | | | | | | | |
| Streptococcus | 942 | macrolide^{R} | ≦0.12 | ≦0.12 | 4 | ≦0.12 | 0.5 |
| pyogenes | | | | | | | |

¹VSE—vancomycin-sensitive Enterococcus
²CLSI—Clinical and Laboratory Standards Institute
³QC—Quality Control
⁴LZD—linezolid
⁵Van—vancomycin
⁶DAP—daptomycin
⁷MSSA—methicillin-sensitive Staphylococcus aureus
⁸MRSA—methicillin-resistant Staphylococcus aureus
⁹FA—fusidic acid
¹⁰VISA—vancomycin-intermediate Staphylococcus aureus
¹¹GM—gentamicin
¹²CHL—chloramphenicol
¹³RA—rifampin
¹⁴MSSE—methicillin-sensitive Staphylococcus epidermidis
¹⁵MRSE—methicillin-resistant Staphylococcus epidermidis
¹⁶mefA—macrolide resistance via efflux
¹⁷ermB—ribosomal erythromycin resistance

The quality control strain MIC data (Table 2) demonstrated that daptomycin, oxacillin, and vancomycin had MIC results within the CLSI quality control ranges for each, thereby validating the assay results for these agents. However, linezolid demonstrated MIC values one dilution higher than the specified CLSI range for both quality control organisms, therefore, the data for linezolid are not acceptable. Overall, linezolid yielded MIC values higher than typically seen for these organisms, consistent with the out-of-range quality control values. The linezolid data are included in Table 1; however, the values should be viewed with caution.

TABLE 2
Minimal Inhibitory Concentration (MIC) Values for CLSI Quality Control Strains

| | Micromyx | | MIC (μg/mL) | | | | |
|---|---|---|---|---|---|---|---|
| Organism | # | Phenotype | Empedopeptin | Daptomycin | Linezolid | Oxacillin | Vancomycin |
| Staphylococcus | 100^{b} | MSSA; | 4 | 0.5 | 8 | 0.25 | 2 |
| aureus | | CLSI QC | | | | | |
| | | strain | | | | | |
| CLSI | | | | 0.25-1 | 1-4 | 0.12-0.5 | 0.5-2 |
| Recommended | | | | | | | |
| Range | | | | | | | |
| Enterococcus | 101^{c} | VSE; | 4 | 1 | 8 | 16 | 4 |
| faecalis | | CLSI QC | | | | | |
| | | strain | | | | | |
| CLSI | | | | 1-4 | 1-4 | 8-32 | 1-4 |
| Recommended | | | | | | | |
| Range | | | | | | | |

^{a} Clinical and Laboratory Standards Institute (2)
^{b}Staphylococcus aureus ATCC 29213
^{c}Enterococcus faecalis ATCC 29212

The phenotypic characteristics were confirmed for all strains where the subject drug was included in the assay (for example, vancomycin-resistance evident for VRE, etc.). Empedopeptin demonstrated broad activity against Gram-positive bacteria, including strains resistant to other antibacterial agents. Against Enterococci, the range of MIC values was 4-32 μg/mL with most strains inhibited at 8-16 μg/mL. The most sensitive Enterococcal strain was E. faecalis 101 (MIC≈4 μg/mL) and the least sensitive was the daptomycin-resistant strain E. faecium 1721. Empedopeptin demonstrated activity against Van A and Van B Enterococci, as well as the linezolid-resistant strain.

Against staphylococci, Empedopeptin demonstrated MIC values in the range of 0.5-8 μg/mL, with the majority of strains inhibited in the range of 4-8 μg/mL. This included isolates resistant to oxacillin, linezolid, fusidic acid, gentamicin, chloramphenicol, and rifampin as well as intermediate-resistance to vancomycin.

Empedopeptin demonstrated greater potency against Streptococci than Enterococci or Staphylococci, inhibiting all strains of S. pneumoniae in the range of ≦0.12-2 μg/mL. This included strains carrying common quinolone resistance mutations, ermB (ribosomal erythromycin resistance), and mefA (macrolide resistance via efflux). Interestingly, the mefA strain was highly susceptible to Empedopeptin. Empedopeptin was also highly active against S. pyogenes inhibiting both test strains at ≦0.12 μg/mL (including the macrolide-resistant strain).

From these results, Empedopeptin has demonstrated activity against several Gram-positive bacteria; and, more importantly, Empedopeptin also demonstrated broad activity against several different antibiotic-resistant strains of bacteria.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of inhibiting bacterial proliferation comprising:
providing a pharmaceutical composition comprising Empedopeptin or a pharmaceutically acceptable salt thereof,
wherein the bacteria comprises at least one Gram positive strain, and the Gram positive strain is resistant to glycopeptides, aminoglycosides, oxazolidinones, penicillins, macrolides, rifamycins, polypeptides, lipopeptides, chloramphenicol, or any combination thereof.

2. The method of claim 1, wherein the Gram positive strain further comprises Enterococcus faecalis, Staphylococcus aureus, Staphylococcus epidermidis, Streptococcus pneumoniae, Streptococcus pyogenes, or any combination thereof.

3. The method of claim 2, wherein the Gram positive strain is further resistant to at least one of linezolid, oxacillin, vancomycin, daptomycin, erythromycin, methicillin, gentamicin, chloramphenicol, fusidic acid, rifampin, or combinations thereof.

4. The method of claim 1, further comprising providing a second pharmaceutical composition, wherein the second pharmaceutical composition comprises a second antibiotic agent.

5. An isolated nucleotide sequence comprising SEQ ID NO 1.

6. An isolated protein sequence comprising SEQ ID NO 2.

7. An isolated nucleotide sequence comprising SEQ ID NO 3.

8. An isolated protein sequence comprising SEQ ID NO 4.

9. An isolated nucleotide sequence comprising SEQ ID NO 5.

10. An isolated protein sequence comprising SEQ ID NO 6.

11. An isolated nucleotide sequence comprising SEQ ID NO 7

12. An isolated protein sequence comprising SEQ ID NO 8.
